(19) United States
(12) Patent Application Publication
(10) Pub. No.: US 2025/0191683 A1
(43) Pub. Date: Jun. 12, 2025

(54) METHODS AND SYSTEMS FOR CHARACTERIZING AND TREATING A DISEASE

(71) Applicant: Foundation Medicine, Inc., Boston, MA (US)

(72) Inventors: Garrett M. FRAMPTON, Somerville, MA (US); Benjamin KAPLAN, Cambridge, MA (US); Smruthy K. SIVAKUMAR, Everett, MA (US); Ethan S. SOKOL, Somerville, MA (US)

(73) Assignee: Foundation Medicine, Inc., Boston, MA (US)

(21) Appl. No.: 18/975,942

(22) Filed: Dec. 10, 2024

Related U.S. Application Data
(60) Provisional application No. 63/608,693, filed on Dec. 11, 2023.

(51) Int. Cl. G16B 20/20; G16B 20/10; G16H 10/40; G16H 50/20

(52) U.S. Cl. CPC G16B 20/20; G16B 20/10; G16H 10/40; G16H 50/20

(57) ABSTRACT

Methods and systems for analyzing or assigning a disease label for a sample are described. The methods may comprise, for example, receiving test sample characterization data comprising genomic alteration statuses for a test sample; receiving, for database samples, database characterization data; determining similarity scores for the database samples; ranking the database samples based on the similarity scores; selecting from the ranked database samples, a subset of database samples most similar to the test sample; determining an enrichment score for the predetermined disease label based on the subset of database samples and the database sample; and confirming or rejecting the predetermined disease label for the test sample based on the enrichment score.

### CROSS-REFERENCE TO RELATED APPLICATIONS

This application claims priority to U.S. Provisional Patent Application Ser. No. 63/608,693, filed Dec. 11, 2023, the disclosure of which is hereby incorporated by reference in its entirety.

### FIELD OF THE INVENTION

The present disclosure relates generally to methods and systems for characterizing and treating a disease. Such methods may include analyzing or assigning a disease label, and more specifically assigning a disease label based on data characterizing a sample from a subject, such as, but not limited to, genomic profiling data. The characterized disease may then be treated based on the assigned disease label.

### BACKGROUND

Clinical samples from a subject can be used for diagnostic purposes. Traditionally, the use of a sample for diagnosing a subject can be based on a clinician's expert opinion and/or experience. Such expert opinion, however, may not necessarily be informed according to a method that is comprehensive or consistent. For example, the disease label, e.g., diagnosis, of one sample may inadvertently be based on criteria that are inconsistent with those used for diagnosing a different sample-even if the two samples harbor many biological similarities. Accordingly, improved methods are needed for analyzing and assigning the one or more disease labels, e.g., diagnoses, of subjects.

### BRIEF SUMMARY

Disclosed herein are methods and systems for analyzing or assigning a disease label, e.g., a diagnosis. Also described are methods of treating the disease based on the assigned disease label. The analyzing or assigning of the disease label can comprise using characterization data, such as genomic data, that corresponds to a sample deriving from a subject. A test sample, or sample of interest, can be scored, based on its corresponding characterization data, such as its genomic data. The test sample's score can indicate the degree of similarity the test sample has, relative to a sample from a database, i.e., the score can be a similarity score, and the similarity score can be regarding the degree of genomic similarity between the test sample and a database sample. The test sample can be compared to multiple database samples, such that each pairwise comparison between the test sample and a database sample results in a corresponding similarity score. That is, when multiple database samples are compared to the test sample, a plurality of similarity scores can correspond to the test sample. The plurality of similarity scores can be ranked and then quantified, such that the degree of representation of the original disease label among the top similarity scores versus the rest of the similarity scores, can be ascertained via numerical and/or statistical methods. Based on the ranking and quantifying, the original disease label, e.g., diagnosis, can be confirmed or rejected. Further systematic comparisons of the test sample against the database of samples can be used to inform whether an alternative disease label, e.g., alternative diagnosis, may be better suited, for the test sample or sample of interest.

In some aspects, disclosed herein is a method for detecting a disease type comprising: providing nucleic acid molecules obtained from a test sample from a subject; ligating adapters onto the nucleic acid molecules; amplifying the ligated nucleic acid molecules; capturing the amplified nucleic acid molecules; capturing the amplified nucleic acid molecules; sequencing, by a sequencer, the captured nucleic acid molecules to obtain sequence reads that represent the captured nucleic acid molecules; receiving, by one or more processors, test sample characterization data comprising genomic alteration statuses for a test sample, wherein the test sample can have a predetermined disease label; receiving, by the one or more processors, for database samples, database characterization data, wherein at least one database sample in the database samples can have a predetermined disease label; determining, by the one or more processors, similarity scores for the database samples, wherein the similarity scores can indicate similarities between the test sample characterization data and the database characterization data; ranking, by the one or more processors, the database samples based on the similarity scores, to generate ranked database samples; selecting, by the one or more processors, from the ranked database samples, a subset of database samples most similar to the test sample; determining, by the one or more processors, an enrichment score for the predetermined disease label based on the subset of database samples and the database samples; and confirming or rejecting, by the one or more processors, the predetermined disease label for the test sample based on the enrichment score.

In some aspects, disclosed herein is a method for detecting a disease type comprising: receiving, by one or more processors, test sample characterization data comprising genomic alteration statuses for a test sample, wherein the test sample can have a predetermined disease label; receiving, by the one or more processors, for database samples, database characterization data, wherein at least one database sample in the database samples can have a predetermined disease label; determining, by the one or more processors, similarity scores for the database samples, wherein the similarity scores can indicate similarities between the test sample characterization data and the database characterization data; ranking, by the one or more processors, the database samples based on the similarity scores, to generate ranked database samples; selecting, by the one or more processors, from the ranked database samples, a subset of database samples most similar to the test sample; determining, by the one or more processors, an enrichment score for the predetermined disease label based on the subset of database samples and the database samples; and confirming or rejecting, by the one or more processors, the predetermined disease label for the test sample based on the enrichment score.

In any of the embodiments herein, the disclosed methods can further comprise: excluding, by the one or more processors, for the test sample, a genomic alteration status from the test sample characterization data, to generate reduced test sample characterization data; excluding, by the one or more processors, for the database samples, the genomic alteration status from the database characterization data, to generate reduced database characterization data; determining, by the one or more processors, second similarity scores, wherein the second similarity scores can indicate similarities between the reduced test sample characterization data, and for a corresponding database sample, the reduced database characterization data; ranking, by the one or more processors, the database samples based on the second similarity scores, to generate second ranked database samples; selecting, by the one or more processors, from the second ranked database samples, a second subset of database samples most similar to the test sample; determining, by the one or more processors, a second enrichment score for the predetermined disease label based on the second subset of database samples and the database samples; and confirming or rejecting, by the one or more processors, the predetermined disease label for the test sample based on the enrichment score and the second enrichment score.

In any of the embodiments herein, the disclosed methods can further comprise determining, by the one or more processors, one or more alternate enrichment scores for one or more alternate disease labels based on the subset of database samples and the database samples; and rejecting, by the one or more processors, the predetermined disease label and assigning an alternate disease label for the test sample based on the enrichment score.

In some embodiments, the alternate disease label can be a type of cancer. In some embodiments, the alternate disease label can indicate that the disease is unknown. In some embodiments, the disease can be unknown, when the number of database samples in the ranked database samples is less than or equal to a predetermined number of database samples. In any of the embodiments herein, the alternate disease label is a cancer of unknown primary. In any of the embodiments herein, the subject can be suspected of having or is determined to have cancer.

In some embodiments, the cancer can be a B cell cancer (multiple myeloma), a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.

In some embodiments, the cancer can comprise acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR/MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma, Hodgkin lymphoma, juvenile idiopathic arthritis, lupus erythematosus, a mantle cell lymphoma, medullary thyroid cancer, melanoma, a melanoma with a BRAF V600 mutation, a melanoma with a BRAF V600E or V600K mutation, Merkel cell carcinoma, multicentric Castleman's disease, multiple hematologic malignancies including Philadelphia chromosome-positive ALL and CML, multiple myeloma, myelofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROS1 gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, rheumatoid arthritis, a small lymphocytic lymphoma, a soft tissue sarcoma, a solid tumor (MSI-H/dMMR), a squamous cell cancer of the head and neck, a squamous non-small cell lung cancer, thyroid cancer, a thyroid carcinoma, urothelial cancer, a urothelial carcinoma, or Waldenstrom's macroglobulinemia.

In some embodiments, the methods disclosed herein can further comprise treating the subject with an anti-cancer therapy. In some embodiments, the anti-cancer therapy can comprise a targeted anti-cancer therapy. In some embodiments, the targeted anti-cancer therapy can comprise abemaciclib (Verzenio), abiraterone acetate (Zytiga), acalabrutinib (Calquence), ado-trastuzumab emtansine (Kadcyla), afatinib dimaleate (Gilotrif), aldesleukin (Proleukin), alectinib (Alecensa), alemtuzumab (Campath), alitretinoin (Panretin), alpelisib (Piqray), amivantamab-vmjw (Rybrevant), anastrozole (Arimidex), apalutamide (Erleada), asciminib hydrochloride (Scemblix), atezolizumab (Tecentriq), avapritinib (Ayvakit), avelumab (Bavencio), axicabtagene ciloleucel (Yescarta), axitinib (Inlyta), belantamab mafodotin-blmf (Blenrep), belimumab (Benlysta), belinostat (Beleodaq), belzutifan (Welireg), bevacizumab (Avastin), bexarotene (Targretin), binimetinib (Mektovi), blinatumomab (Blincyto), bortezomib (Velcade), bosutinib (Bosulif), brentuximab vedotin (Adcetris), brexucabtagene autoleucel (Tecartus), brigatinib (Alunbrig), cabazitaxel (Jevtana), cabozantinib (Cabometyx), cabozantinib (Cabometyx, Cometriq), canakinumab (Ilaris), capmatinib hydrochloride (Tabrecta), capivasertib, carfilzomib (Kyprolis), cemiplimab-rwlc (Libtayo), ceritinib (LDK378/Zykadia), cetuximab (Erbitux), cobimetinib (Cotellic), copanlisib hydrochloride (Aligopa), crizotinib (Xalkori), dabrafenib (Tafinlar), dacomitinib (Vizimpro), daratumumab (Darzalex), daratumumab and hyaluronidase-fihj (Darzalex Faspro), darolutamide (Nubega), dasatinib (Sprycel), denileukin diftitox (Ontak), denosumab (Xgeva), dinutuximab (Unituxin), dostarlimab-gxly (Jemperli), durvalumab (Imfinzi), duvelisib (Copiktra), elotuzumab (Empliciti), enasidenib mesylate (Idhifa), encorafenib (Braftovi), enfortumab vedotin-ejfv (Padcev), entrectinib (Rozlytrek), enzalutamide (Xtandi), erdafitinib (Balversa), erlotinib (Tarceva), everolimus (Afinitor), exemestane (Aromasin), fam-trastuzumab deruxtecan-nxki (Enhertu), fedratinib hydrochloride (Inrebic), fulvestrant (Faslodex), gefitinib (Iressa), gemtuzumab ozogamicin (Mylotarg), gilteritinib (Xospata), glasdegib maleate (Daurismo), hyaluronidase-zzxf (Phesgo), ibrutinib (Imbruvica), ibritumomab tiuxetan (Zevalin), idecabtagene vicleucel (Abecma), idelalisib (Zydelig), imatinib mesylate (Gleevec), infigratinib phosphate (Truseltiq), inotuzumab ozogamicin (Besponsa), iobenguane I131 (Azedra), ipilimumab (Yervoy), isatuximab-irfc (Sarclisa), ivosidenib (Tibsovo), ixazomib citrate (Ninlaro), lanreotide acetate (Somatuline Depot), lapatinib (Tykerb), larotrectinib sulfate (Vitrakvi), lenvatinib mesylate (Lenvima), letrozole (Femara), lisocabtagene maraleucel (Breyanzi), loncastuximab tesirine-lpyl (Zynlonta), lorlatinib (Lorbrena), lutetium Lu 177-dotatate (Lutathera), margetuximab-cmkb (Margenza), midostaurin (Rydapt), mobocertinib succinate (Exkivity), mogamulizumab-kpkc (Poteligeo), moxetumomab pasudotox-tdfk (Lumoxiti), naxitamab-gqgk (Danyelza), necitumumab (Portrazza), neratinib maleate (Nerlynx), nilotinib (Tasigna), niraparib tosylate monohydrate (Zejula), nivolumab (Opdivo), obinutuzumab (Gazyva), ofatumumab (Arzerra), olaparib (Lynparza), olaratumab (Lartruvo), osimertinib (Tagrisso), palbociclib (Ibrance), panitumumab (Vectibix), panobinostat (Farydak), pazopanib (Votrient), pembrolizumab (Keytruda), pemigatinib (Pemazyre), pertuzumab (Perjeta), pexidartinib hydrochloride (Turalio), polatuzumab vedotin-piiq (Polivy), ponatinib hydrochloride (Iclusig), pralatrexate (Folotyn), pralsetinib (Gavreto), radium 223 dichloride (Xofigo), ramucirumab (Cyramza), regorafenib (Stivarga), ribociclib (Kisqali), ripretinib (Qinlock), rituximab (Rituxan), rituximab and hyaluronidase human (Rituxan Hycela), romidepsin (Istodax), rucaparib camsylate (Rubraca), ruxolitinib phosphate (Jakafi), sacituzumab govitecan-hziy (Trodelvy), seliciclib, selinexor (Xpovio), selpercatinib (Retevmo), selumetinib sulfate (Koselugo), siltuximab (Sylvant), sipuleucel-T (Provenge), sirolimus protein-bound particles (Fyarro), sonidegib (Odomzo), sorafenib (Nexavar), sotorasib (Lumakras), sunitinib (Sutent), tafasitamab-cxix (Monjuvi), tagraxofusp-erzs (Elzonris), talazoparib tosylate (Talzenna), tamoxifen (Nolvadex), tazemetostat hydrobromide (Tazverik), tebentafusp-tebn (Kimmtrak), temsirolimus (Torisel), tepotinib hydrochloride (Tepmetko), tisagenlecleucel (Kymriah), tisotumab vedotin-tftv (Tivdak), tocilizumab (Actemra), tofacitinib (Xeljanz), tositumomab (Bexxar), trametinib (Mekinist), trastuzumab (Herceptin), tretinoin (Vesanoid), tivozanib hydrochloride (Fotivda), toremifene (Fareston), tucatinib (Tukysa), umbralisib tosylate (Ukoniq), vandetanib (Caprelsa), vemurafenib (Zelboraf), venetoclax (Venclexta), vismodegib (Erivedge), vorinostat (Zolinza), zanubrutinib (Brukinsa), ziv-aflibercept (Zaltrap), or any combination thereof.

In any of the embodiments herein, the method can further comprise obtaining the test sample from the subject. In any of the embodiments herein, the test sample can comprise a tissue biopsy sample, a liquid biopsy sample, or a normal control. In some embodiments, the test sample can be a liquid biopsy sample and can comprise blood, plasma, cerebrospinal fluid, sputum, stool, urine, or saliva. In some embodiments, the test sample can be a liquid biopsy sample and can comprise circulating tumor cells (CTCs). In some embodiments, the test sample can be a liquid biopsy sample and can comprise cell-free DNA (cfDNA), circulating tumor DNA (ctDNA), or any combination thereof. In any of the embodiments herein, the plurality of nucleic acid molecules can comprise a mixture of tumor nucleic acid molecules and non-tumor nucleic acid molecules. In some embodiments, the tumor nucleic acid molecules can be derived from a tumor portion of a heterogeneous tissue biopsy sample, and the non-tumor nucleic acid molecules are derived from a normal portion of the heterogeneous tissue biopsy sample.

In some embodiments, the sample can comprise a liquid biopsy sample, and wherein the tumor nucleic acid molecules can be derived from a circulating tumor DNA (ctDNA) fraction of the liquid biopsy sample, and the non-tumor nucleic acid molecules are derived from a non-tumor, cell-free DNA (cfDNA) fraction of the liquid biopsy sample. In any of the embodiments herein, the one or more adapters can comprise amplification primers, flow cell adaptor sequences, substrate adapter sequences, or sample index sequences. In any of the embodiments herein, the captured nucleic acid molecules can be captured from the amplified nucleic acid molecules by hybridization to one or more bait molecules. In some embodiments, the one or more bait molecules can comprise one or more nucleic acid molecules, each comprising a region that is complementary to a region of a captured nucleic acid molecule. In any of the embodiments herein, amplifying nucleic acid molecules can comprise performing a polymerase chain reaction (PCR) amplification technique, a non-PCR amplification technique, or an isothermal amplification technique. In any of the embodiments herein, the sequencing can comprise use of a massively parallel sequencing (MPS) technique, whole genome sequencing (WGS), whole exome sequencing, targeted sequencing, direct sequencing, or Sanger sequencing technique. In some embodiments, the sequencing can comprise massively parallel sequencing, and the massively parallel sequencing technique comprises next generation sequencing (NGS). In any of the embodiments herein, the sequencer can comprise a next generation sequencer. In any of the embodiments herein, one or more of the plurality of sequencing reads overlap one or more gene loci within one or more subgenomic intervals in the sample.

In some embodiments, the one or more gene loci comprises between 10 and 20 loci, between 10 and 40 loci, between 10 and 60 loci, between 10 and 80 loci, between 10 and 100 loci, between 10 and 150 loci, between 10 and 200 loci, between 10 and 250 loci, between 10 and 300 loci, between 10 and 350 loci, between 10 and 400 loci, between 10 and 450 loci, between 10 and 500 loci, between 20 and 40 loci, between 20 and 60 loci, between 20 and 80 loci, between 20 and 100 loci, between 20 and 150 loci, between 20 and 200 loci, between 20 and 250 loci, between 20 and 300 loci, between 20 and 350 loci, between 20 and 400 loci, between 20 and 500 loci, between 40 and 60 loci, between 40 and 80 loci, between 40 and 100 loci, between 40 and 150 loci, between 40 and 200 loci, between 40 and 250 loci, between 40 and 300 loci, between 40 and 350 loci, between 40 and 400 loci, between 40 and 500 loci, between 60 and 80 loci, between 60 and 100 loci, between 60 and 150 loci, between 60 and 200 loci, between 60 and 250 loci, between 60 and 300 loci, between 60 and 350 loci, between 60 and 400 loci, between 60 and 500 loci, between 80 and 100 loci, between 80 and 150 loci, between 80 and 200 loci, between 80 and 250 loci, between 80 and 300 loci, between 80 and 350 loci, between 80 and 400 loci, between 80 and 500 loci, between 100 and 150 loci, between 100 and 200 loci, between 100 and 250 loci, between 100 and 300 loci, between 100 and 350 loci, between 100 and 400 loci, between 100 and 500 loci, between 150 and 200 loci, between 150 and 250 loci, between 150 and 300 loci, between 150 and 350 loci, between 150 and 400 loci, between 150 and 500 loci, between 200 and 250 loci, between 200 and 300 loci, between 200 and 350 loci, between 200 and 400 loci, between 200 and 500 loci, between 250 and 300 loci, between 250 and 350 loci, between 250 and 400 loci, between 250 and 500 loci, between 300 and 350 loci, between 300 and 400 loci, between 300 and 500 loci, between 350 and 400 loci, between 350 and 500 loci, or between 400 and 500 loci.

In any of the embodiments herein, the one or more gene loci can comprise ABL1, ACVR1B, AKT1, AKT2, AKT3, ALK, ALOX12B, AMER1, APC, AR, ARAF, ARFRP1, ARID1A, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKN1A, CDKN1B, CDKN2A, CDKN2B, CDKN2C, CEBPA, CHEK1, CHEK2, CIC, CREBBP, CRKL, CSF1R, CSF3R, CTCF, CTNNA1, CTNNB1, CUL3, CUL4A, CXCR4, CYP17A1, DAXX, DDR1, DDR2, DIS3, DNMT3A, DOTIL, EED, EGFR, EMSY (C11orf30), EP300, EPHA3, EPHB1, EPHB4, ERBB2, ERBB3, ERBB4, ERCC4, ERG, ERRFI1, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNF1A, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MST1R, MTAP, MTOR, MUTYH, MYB, MYC, MYCL, MYCN, MYD88, NBN, NF1, NF2, NFE2L2, NFKBIA, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCD1LG2, PDGFRA, PDGFRB, PDK1, PIK3C2B, PIK3C2G, PIK3CA, PIK3CB, PIK3R1, PIM1, PMS2, POLD1, POLE, PPARG, PPP2R1A, PPP2R2A, PRDM1, PRKAR1A, PRKCI, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSCILl, WT1, XPO1, XRCC2, ZNF217, ZNF703, or any combination thereof.

In any of the embodiments herein, the one or more gene loci can comprise ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CS1, CTLA-4, dMMR, EGFR, ERBB1, ERBB2, FGFR1-3, FLT3, GD2, HDAC, HER1, HER2, HR, IDH2, IL-1β, IL-6, IL-6R, JAK1, JAK2, JAK3, KIT, KRAS, MEK, MET, MSI-H, mTOR, PARP, PD-1, PDGFR, PDGFRα, PDGFRβ, PD-L1, PI3Kδ, PIGF, PTCH, RAF, RANKL, RET, ROS1, SLAMF7, VEGF, VEGFA, VEGFB, or any combination thereof.

In any of the embodiments herein, the methods disclosed herein can further comprise generating, by the one or more processors, a report indicating the predetermined disease label or assigning the alternate disease label to the test sample. In some embodiments, the disclosed methods can further comprise transmitting the report to a healthcare provider. In some embodiments, the report can be transmitted via a computer network or a peer-to-peer connection. In any of the embodiments herein, the alternate disease label can be a cancer. In any of the embodiments herein, the predetermined disease label or the alternate disease label can be a cancer of unknown primary.

In any of the embodiments herein, selecting from the genomic alteration statuses can comprise pathogenic genomic alteration statuses. In any of the embodiments herein, the one or more of the determined similarity scores or one or more of the determined second similarity scores can increase by a predetermined pathogenic short variant scoring value, when the test sample and the corresponding database sample share a pathogenic short variant affecting a same gene. In any of the embodiments herein, one or more of the determined similarity scores or one or more of the determined second similarity scores can increase by a predetermined same pathogenic effect scoring value, when the test sample and the corresponding database sample can share a pathogenic short variant affecting a same gene with identical protein effects.

In any of the embodiments herein, one or more of the determined similarity scores or one or more of the determined second similarity scores increase by a predetermined pathogenic copy number amplification scoring value, when the test sample and the corresponding database sample share a pathogenic copy number amplification occurring on a same amplicon segment. In any of the embodiments herein, one or more of the determined similarity scores or one or more of the determined second similarity scores increases by a predetermined pathogenic copy number deletion scoring value, when the test sample and the corresponding database sample share a pathogenic copy number deletion occurring on a same commonly deleted segment.

In any of the embodiments herein, wherein one or more of the determined similarity scores or one or more of the determined second similarity scores can increase by a predetermined pathogenic rearrangement scoring value, when the test sample and the corresponding database sample share a same two gene partners in a pathogenic rearrangement. In any of the embodiments herein, wherein one or more of the determined similarity scores or one or more of the determined second similarity scores can increase by a predetermined same gene partner pathogenic rearrangement scoring value, when the test sample and the corresponding database sample can share a same one gene partner in a pathogenic rearrangement.

In any of the embodiments herein, one or more of the determined similarity scores or one or more of the determined second similarity scores can decrease by a predetermined non-common genomic alteration status scoring value, when the test sample and the corresponding database sample may not share a same genomic alteration status from the genomic alteration statuses.

In any of the embodiments herein, one or more of the determined similarity scores or one or more of the determined second similarity scores can increase by a predetermined tumor mutational burden (TMB) scoring value, when the test sample and the corresponding database sample can each have a TMB score above a predetermined TMB score threshold. In any of the embodiments herein, one or more of the determined similarity scores or one or more of the determined second similarity scores can increase by a predetermined dominant mutational signature scoring value, when the test sample and the one database sample can share a dominant mutational signature. In some embodiments, the dominant mutational signature can be associated with exposure to an alkylating agent, tobacco, or ultraviolet light, or an altered activity of APOBEC, a mutation in one or more mismatch repair pathway genes, a mutation in a POLE gene, or a mutation in BRCA1 or BRCA2. In any of the embodiments herein, one or more of the determined similarity scores or one or more of the determined second similarity scores can increase by a predetermined high TMB and dominant mutational signature scoring value, when the test sample and the corresponding database sample can share both the high TMB score and the dominant mutational signature. In any of the embodiments herein, the one or more determined similarity scores or the one or more determined second similarity scores can increase by a predetermined copy number signature scoring value, when the test sample and the corresponding database sample can share a copy number signature.

In any of the embodiments herein, the one or more determined similarity scores or the one or more determined second similarity scores can increase by a predetermined aneuploidy feature scoring value, when the test sample and the corresponding database sample share a common aneuploidy feature. In any of the embodiments herein, all the database samples from the ranked database samples can have the determined similarity score or the determined second similarity score of at least a minimum score.

In any of the embodiments herein, database samples corresponding to a predetermined number of most similar database samples can be used for determining the enrichment score or the second enrichment score. In any of the embodiments herein, the enrichment score or the second enrichment score can be an odds ratio. In any of the embodiments herein, the odds ratio can be determined by Fisher's exact test. In any of the embodiments herein, the enrichment score or the second enrichment score can be a U score from a Mann Whitney U-test. In any of the embodiments herein, the methods disclosed herein can further comprise determining a confidence value indicating whether the predetermined disease label can be correctly confirmed or rejected. In some embodiments, the confidence value can be a p-value determined by the Fisher's exact test.

In any of the embodiments herein, the predetermined disease label can be rejected, when the enrichment score for the predetermined disease label can be less than or equal to a first predetermined enrichment score threshold. In any of the embodiments herein, the predetermined disease label can be rejected, when the confidence value can be less than or equal to a first predetermined confidence value threshold. In any of the embodiments herein, the predetermined disease label can be rejected, when the enrichment score for the predetermined disease label can be less than or equal to the first predetermined enrichment score threshold and the confidence value can be less than or equal to the first predetermined confidence value threshold.

In any of the embodiments herein, the alternate disease label can be accepted, when the enrichment score for the alternate disease label can be greater than or equal to a second predetermined enrichment score threshold. In any of the embodiments herein, the alternate disease label can be accepted, when the confidence value can be less than or equal to a second predetermined confidence value threshold. In any of the embodiments herein, the alternate disease label can be accepted, when the enrichment score for the predetermined disease label can be greater than or equal to a second predetermined enrichment score threshold and the confidence value can be less than or equal to the second predetermined confidence value threshold.

In some aspects, disclosed herein is a method for diagnosing a disease, the method comprising diagnosing that the subject has a disease based on confirming or rejecting of the predetermined disease label, wherein the confirming or rejecting of the predetermined disease label can be according to the method of any of the embodiments disclosed herein. In some aspects, disclosed herein is a method for diagnosing a disease, the method comprising diagnosing that the subject has a disease based on assigning the alternate disease label, wherein the assigning the alternate disease label can be according to the method of any of the embodiments disclosed herein.

In some aspects, disclosed herein is a method of selecting an anti-cancer therapy, the method comprising: responsive to confirming a predetermined disease label for the cancer according to the method of any of the embodiments described herein, selecting an anti-cancer therapy effective in treating the cancer. In some aspects, disclosed herein is a method of selecting an anti-cancer therapy, the method comprising: responsive to assigning an alternate disease label for the cancer according to the methods described herein, selecting an anti-cancer therapy effective in treating the cancer.

In some aspects, disclosed herein is a method of treating a cancer in a subject, comprising: responsive to confirming a predetermined disease label for the cancer according to the method of any of the embodiments disclosed herein, administering to the subject an anti-cancer therapy effective in treating the cancer. In some aspects, disclosed herein is a method of treating a cancer in a subject, comprising: responsive to assigning an alternate disease label for the cancer according to the method of any of the embodiments disclosed herein, administering to the subject an anti-cancer therapy effective in treating the cancer.

In some aspects, disclosed herein is a method for monitoring cancer progression or recurrence in a subject, the method comprising: confirming or rejecting a first predetermined disease label or assigning a first alternate disease label in a first sample obtained from the subject at a first time point according to the method of any of the embodiments disclosed herein; confirming or rejecting a second predetermined disease label or assigning a second alternate disease label in a second sample obtained from the subject at a second time point, and comparing the first predetermined disease label or the first alternate disease label to the second predetermined disease label or the second alternate disease label, thereby monitoring the cancer progression or recurrence.

In some embodiments, the second predetermined disease label or the second alternate disease label for the second sample can be determined according to the method of any of the embodiments disclosed herein.

In any of the embodiments herein, the disclosed methods can further comprise selecting an anti-cancer therapy for the subject in response to the cancer progression. In any of the embodiments herein, the disclosed methods can further comprise administering an anti-cancer therapy to the subject in response to the cancer progression. In any of the embodiments herein, the disclosed methods can further comprise adjusting an anti-cancer therapy for the subject in response to the cancer progression. In any of the embodiments herein, the disclosed methods can further comprise adjusting a dosage of the anti-cancer therapy or selecting a different anti-cancer therapy in response to the cancer progression. In some embodiments, the disclosed methods can further comprise administering the adjusted anti-cancer therapy to the subject. In any of the embodiments herein, the first time point can be before the subject has been administered an anti-cancer therapy, and wherein the second time point can be after the subject has been administered the anti-cancer therapy.

In any of the embodiments herein, the subject can have a cancer, can be at risk of having a cancer, can be routine tested for cancer, or can be suspected of having a cancer. In any of the embodiments herein, the cancer can be a solid tumor. In any of the embodiments herein, the cancer can be a hematological cancer. In any of the embodiments herein, the anti-cancer therapy can comprise chemotherapy, radiation therapy, immunotherapy, a targeted therapy, or surgery.

In any of the embodiments herein, the disclosed methods can further comprise determining, identifying, or applying the predetermined disease label as a diagnostic value associated with the sample. In any of the embodiments herein, the disclosed methods can further comprise determining, identifying, or applying the alternate disease label as a diagnostic value associated with the sample. In any of the embodiments herein, the disclosed methods can further comprise generating a genomic profile for the subject based on confirming or rejecting the predetermined disease label. In any of the embodiments herein, the disclosed methods can further comprise generating a genomic profile for the subject based on assigning the alternate disease label. In some embodiments, the genomic profile for the subject further can comprise results from a comprehensive genomic profiling (CGP) test, a gene expression profiling test, a cancer hotspot panel test, a DNA methylation test, a DNA fragmentation test, an RNA fragmentation test, or any combination thereof. In any of the embodiments herein, the genomic profile for the subject can further comprise results from a nucleic acid sequencing-based test.

In any of the embodiments herein, the disclosed methods can further comprise selecting an anti-cancer therapy, administering an anti-cancer therapy, or applying an anti-cancer therapy to the subject based on the generated genomic profile. In any of the embodiments herein, the confirming or rejecting of the predetermined disease label for the sample can be used in making suggested treatment decisions for the subject. In any of the embodiments herein, the assigning the alternate disease label for the sample can be used in making suggested treatment decisions for the subject. In any of the embodiments herein, the confirming or rejecting of the predetermined disease label for the sample can be used in applying or administering a treatment to the subject. In any of the embodiments herein, the assigning the alternate disease label for the sample can be used in applying or administering a treatment to the subject.

In some aspects, disclosed herein is a system comprising: one or more processors; and a memory communicatively coupled to the one or more processors and configured to store instructions that, when executed by the one or more processors, cause the system to: receive test sample characterization data comprising genomic alteration statuses for a test sample, wherein the test sample can have a predetermined disease label; receive for database samples, database characterization data, wherein at least one database sample in the database samples can have a predetermined disease label; determine similarity scores for the database samples, wherein the similarity scores can indicate similarities between the test sample characterization data and the database characterization data; rank the database samples based on the similarity scores, to generate ranked database samples; select from the ranked database samples, a subset of database samples most similar to the test sample; determine an enrichment score for the predetermined disease label based on the subset of database samples and the database samples; and confirm or reject the predetermined disease label for the test sample based on the enrichment score.

In some embodiments, the disclosed methods can further comprise instructions that, when executed by the one or more processors, can cause the system to: determine one or more alternate enrichment scores for one or more alternate disease labels based on the subset of database samples and the database samples; and reject the predetermined disease label and assign an alternate disease label for the test sample based on the enrichment score.

In any of the embodiments herein, the disclosed methods can further comprise instructions that, when executed by the one or more processors, cause the system to: exclude, for the test sample, a genomic alteration status from the test sample characterization data, to generate reduced test sample characterization data; exclude, for the database samples, the genomic alteration status from the database characterization data, to generate reduced database characterization data; determine second similarity scores, wherein the second similarity scores indicate similarities between the reduced test sample characterization data, and for a corresponding database sample, the reduced database characterization data; rank the database samples based on the second similarity scores, to generate second ranked database samples; select from the second ranked database samples, a second subset of database samples most similar to the test sample; determine a second enrichment score for the predetermined disease label based on the second subset of database samples and the database samples; and confirm or reject the predetermined disease label for the test sample based on the enrichment score and the second enrichment score.

In some aspects, disclosed herein is a non-transitory computer-readable storage system storing one or more programs, the one or more programs comprising instructions, which when executed by one or more processors of a system, can cause the system to: receive test sample characterization data comprising genomic alteration statuses for a test sample, wherein the test sample has a predetermined disease label; receive for database samples, database characterization data, wherein at least one database sample in the database samples has a predetermined disease label; determine similarity scores for the database samples, wherein the similarity scores indicate similarities between the test sample characterization data and the database characterization data; rank the database samples based on the similarity scores, to generate ranked database samples; select from the ranked database samples, a subset of database samples most similar to the test sample; determine an enrichment score for the predetermined disease label based on the subset of database samples and the database samples; and confirm or reject the predetermined disease label for the test sample based on the enrichment score.

In some embodiments, the non-transitory computer-readable storage medium can further comprise instructions that, when executed by the one or more processors, cause the system to: determine one or more alternate enrichment scores for one or more alternate disease labels based on the subset of database samples and the database samples; and can reject the predetermined disease label and can assign an alternate disease label for the test sample based on the enrichment score.

In any of the embodiments herein, the non-transitory computer-readable storage medium can further comprise instructions that, when executed by the one or more processors, can cause the system to: exclude, for the test sample, a genomic alteration status from the test sample characterization data, to generate reduced test sample characterization data; exclude, for the database samples, the genomic alteration status from the database characterization data, to generate reduced database characterization data; determine second similarity scores, wherein the second similarity scores can indicate similarities between the reduced test sample characterization data, and for a corresponding database sample, the reduced database characterization data; rank the database samples based on the second similarity scores, to generate second ranked database samples; select from the second ranked database samples, a second subset of database samples most similar to the test sample; determine a second enrichment score for the predetermined disease label based on the second subset of database samples and the database samples; and confirm or reject the predetermined disease label for the test sample based on the enrichment score and the second enrichment score.

### INCORPORATION BY REFERENCE

All publications, patents, and patent applications mentioned in this specification are herein incorporated by reference in their entirety to the same extent as if each individual publication, patent, or patent application was specifically and individually indicated to be incorporated by reference in its entirety. In the event of a conflict between a term herein and a term in an incorporated reference, the term herein controls.

### BRIEF DESCRIPTION OF THE DRAWINGS

Various aspects of the disclosed methods, devices, and systems are set forth with particularity in the appended claims. A better understanding of the features and advantages of the disclosed methods, devices, and systems will be obtained by reference to the following detailed description of illustrative embodiments and the accompanying drawings, of which:

FIG. 1 indicates a non-limiting exemplary method for analyzing or providing a disease label, e.g., diagnosis, for a subject.

FIG. 2 indicates a non-limiting exemplary schematic of a score from a test sample being compared, pairwise, to the scores of database samples.

FIG. 3 indicates a non-limiting exemplary schematic of scores from database samples being used to analyze or assign a disease label, e.g., a diagnosis, for a subject.

FIG. 4 depicts an exemplary computing device or system in accordance with one embodiment of the present disclosure.

FIG. 5 depicts an exemplary computer system or computer network, in accordance with some instances of the systems described herein.

FIG. 6 indicates a non-limiting example of data depicting the composition of cancer types for a database.

FIG. 7A indicates a non-limiting example of data depicting the number and percentage of misdiagnoses, in a table, for various disease groups.

FIG. 7B indicates a non-limiting example of data depicting the number of misdiagnoses, in a stacked bar chart, for various disease groups.

FIG. 8 indicates a non-limiting example of data depicting original and alternative diagnoses for subjects, based on samples.

### DETAILED DESCRIPTION

Methods and systems for analyzing or assigning a disease label are described. Such methods and systems may be used, for example, to detect or confirm a disease type for a subject. Further, the subject may be treated based on the detected or confirmed type of disease. Test sample characterization data, including genomic alteration statuses, can be received, and the test sample can have a disease label. Database characterization data can be received for database samples, and the database samples can also have disease labels. Similarity scores for the database samples can be determined, and the similarity scores can indicate similarities between the test sample characterization data and the database characterization data. The database samples can be ranked based on the similarity scores. A subset of database samples most similar to the test sample can then be selected from the ranked database samples. An enrichment score for the disease label can be determined, based on the subset of database samples and the entirety of the database samples. The disease label for the test sample can be confirmed or rejected, based on the enrichment score. The subset of database samples and the entirety of the database samples can be used to also determine alternate enrichment scores for alternate disease labels. The disease label can be rejected and an alternate disease label can be assigned for the test sample, based on the enrichment score.

Clinical samples from a subject can be used for diagnostic purposes. For example, a clinician's expert opinion and/or experience can be used to assign a disease label, e.g., a diagnosis, to a sample and the subject from which the sample derives. The assigning of disease labels, however, can be inconsistent. For example, diagnosing samples and subjects may require a subjective assessment from the expert clinician, and can be prone to human error. In addition, when assigning a disease label to a new test sample, the clinician may not necessarily recall or be aware of past similar samples and their corresponding diagnoses. The lack of recollection or awareness may be especially true if the number of past similar samples is large, or if many of the past similar samples were analyzed further in the past. As a result, the clinician may inadvertently assign to the sample, a disease label that is inconsistent with the history of assigned disease labels for other samples. The inconsistent assigning of disease labels to samples can prove highly problematic for future assignments. Pre-existing incorrect diagnoses may provide a weaker expectation for correct assignments in the future, and thus, each incorrect assignment may exacerbate, in the future, the assigning of correct or consistent disease labels. The methods and systems described herein aim to rectify the inconsistent assigning of disease labels, e.g., diagnoses. In some instances, the methods and systems described herein can correct a previously assigned, i.e., predetermined disease label, can confirm a previously assigned disease label, or can determine a disease label when previously a disease label did not exist.

The methods and systems described herein can use the correctly assigned disease label to provide or inform more appropriate treatments. For example, a patient that was originally assigned a prostate cancer diagnosis but was then corrected to having a kidney cancer diagnosis, according to the methods disclosed herein, can then be provided therapies directed to kidney cancer, rather than therapies directed to prostate cancer. In some scenarios, the therapeutic avenues associated with the original, e.g., incorrect, disease label may be less effective or even non-effective, relative to a therapy typical of the new corrected disease label. The ability to provide improved e.g., more appropriate therapies, based on improved assigning on disease labels, can sometimes have dramatic effects. For example, some therapies, such as certain pharmaceuticals, may entail harmful side-effects. The methods disclosed herein can help avert the prescription of inappropriate therapeutics that may provide harmful risks to the subject, in addition to providing the more appropriate and worthwhile therapeutics that can be prescribed based on the correct assigning of a disease label, based on the methods described herein.

Described herein is a method for the systematic scoring and classifying of a test sample from a subject. The described methods articulate criteria by which to compare the test sample to a database of samples, e.g., to each sample in a database of samples. The test sample is assigned a similarity score—that is, a score based on the degree of similarity the test sample has to a sample in the database. A similarity score is determined for every database sample to which the test sample is compared. The same criteria are applied to every comparison made between the test sample and a sample from the database. The criteria can be based on genomic similarities, e.g., common mutation types, between the test sample and the database sample. By using the same criteria for each comparison between the test sample and a database sample, the described methods ensure consistency across the assigning of disease labels, e.g., diagnoses. Moreover, in some instances, the methods described herein can be implemented computationally, e.g., on a system such as a non-transitory computer-readable storage system. In doing so, even if the criteria for comparing a test sample and a database sample is intricate and/or extensive, the similarity score for a given comparison can be determined efficiently and consistently. A computational implementation of the described methods can also mitigate inconsistencies when assigning disease labels for more recent samples, e.g., more recently assigned disease labels may inadvertently be assigned according to different criteria than earlier samples, if the methods described herein are not used. The described methods ensure that samples are provided disease labels, according to consistent criteria.

One key advantage of the method described herein is that the method can be used to identify and correct disease labels already assigned to a sample, e.g., the described method can flag and correct misdiagnoses. Identifying a misdiagnosis is achieved by comparing the misdiagnosis against similar samples from the database and analyzing those similar samples' corresponding diagnoses. If, according to statistical methods, the diagnoses associated with the similar database samples are different from the diagnosis associated with the test sample, the diagnosis is flagged as a misdiagnosis. Furthermore, if, according to statistical methods, a diagnosis associated with a similar database sample is a more likely label than the misdiagnosis, a new diagnosis can be proposed. The new diagnosis would be based on the labels of the database samples that most resemble the test sample.

Another key advantage of the methods described herein is its interpretability. The described methods forego the use of black box techniques, such as some machine learning techniques, that aim to generate associations between predictor and response variables, without necessarily divulging a rationale behind the generated associations. The methods discussed herein are based on heuristics that are tied to interpretable features described in the test sample and database sample characterization data, e.g., known biological features, such as genomic alteration statuses, which can include short variants, copy number alterations, etc. The use of an interpretable methodology as articulated by the present disclosure allows for an improved understanding of why a predetermined disease label may be accepted or rejected for a test sample.

The methods described herein improve upon assigning a disease label for a test sample based on a clinician's subjective opinion, by comparing the test sample against a database of samples, and by using common criteria when comparing the test sample against a database sample. The methods described herein can be implemented computationally and can achieve systematic and consistent comparisons between a test sample and a database sample. The described methods can be used to determine a disease label when a disease label did not already exist, confirm a previously assigned disease label, reject a previously assigned label, and/or provide an alternative disease label, e.g., correct a misdiagnosis, according to the applying of consistent criteria and statistical analyses.

In some aspects, disclosed herein is a method for detecting a disease type comprising: receiving, by one or more processors, test sample characterization data comprising genomic alteration statuses for a test sample, wherein the test sample can have a predetermined disease label; receiving, by the one or more processors, for database samples, database characterization data, wherein at least one database sample in the database samples can have a predetermined disease label; determining, by the one or more processors, similarity scores for the database samples, wherein the similarity scores can indicate similarities between the test sample characterization data and the database characterization data; ranking, by the one or more processors, the database samples based on the similarity scores, to generate ranked database samples; selecting, by the one or more processors, from the ranked database samples, a subset of database samples most similar to the test sample; determining, by the one or more processors, an enrichment score for the predetermined disease label based on the subset of database samples and the database samples; and confirming or rejecting, by the one or more processors, the predetermined disease label for the test sample based on the enrichment score. In some aspects, the methods disclosed herein can further comprise: determining, by the one or more processors, one or more alternate enrichment scores for one or more alternate disease labels based on the subset of database samples and the database samples; and rejecting, by the one or more processors, the predetermined disease label and assigning an alternate disease label for the test sample based on the enrichment score.

### Definitions

Unless otherwise defined, all of the technical terms used herein have the same meaning as commonly understood by one of ordinary skill in the art in the field to which this disclosure belongs.

As used in this specification and the appended claims, the singular forms “a”, “an”, and “the” include plural references unless the context clearly dictates otherwise. Any reference to “or” herein is intended to encompass “and/or” unless otherwise stated.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

As used herein, the terms “comprising” (and any form or variant of comprising, such as “comprise” and “comprises”), “having” (and any form or variant of having, such as “have” and “has”), “including” (and any form or variant of including, such as “includes” and “include”), or “containing” (and any form or variant of containing, such as “contains” and “contain”), are inclusive or open-ended and do not exclude additional, un-recited additives, components, integers, elements, or method steps.

As used herein, the terms “individual,” “patient,” or “subject” are used interchangeably and refer to any single animal, e.g., a mammal (including such non-human animals as, for example, dogs, cats, horses, rabbits, zoo animals, cows, pigs, sheep, and non-human primates) for which treatment is desired. In particular embodiments, the individual, patient, or subject herein is a human.

The terms “cancer” and “tumor” are used interchangeably herein. These terms refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist alone within an animal, or can be a non-tumorigenic cancer cell, such as a leukemia cell. These terms include a solid tumor, a soft tissue tumor, or a metastatic lesion. As used herein, the term “cancer” includes premalignant, as well as malignant cancers.

As used herein, “treatment” (and grammatical variations thereof such as “treat” or “treating”) refers to clinical intervention (e.g., administration of an anti-cancer agent or anti-cancer therapy) in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

As used herein, the term “subgenomic interval” (or “subgenomic sequence interval”) refers to a portion of a genomic sequence.

As used herein, the term “subject interval” refers to a subgenomic interval or an expressed subgenomic interval (e.g., the transcribed sequence of a subgenomic interval).

As used herein, the terms “variant sequence” or “variant” are used interchangeably and refer to a modified nucleic acid sequence relative to a corresponding “normal” or “wild-type” sequence. In some instances, a variant sequence may be a “short variant sequence” (or “short variant”), i.e., a variant sequence of less than about 50 base pairs in length.

The terms “allele frequency” and “allele fraction” are used interchangeably herein and refer to the fraction of sequence reads corresponding to a particular allele relative to the total number of sequence reads for a genomic locus.

The terms “variant allele frequency” and “variant allele fraction” are used interchangeably herein and refer to the fraction of sequence reads corresponding to a particular variant allele relative to the total number of sequence reads for a genomic locus.

It is understood that aspects and variations of the invention described herein include “consisting” and/or “consisting essentially of” aspects and variations.

When a range of values is provided, it is to be understood that each intervening value between the upper and lower limit of that range, and any other stated or intervening value in that states range, is encompassed within the scope of the present disclosure. Where the stated range includes upper or lower limits, ranges excluding either of those included limits are also included in the present disclosure.

Some of the analytical methods described herein include mapping sequences to a reference sequence, determining sequence information, and/or analyzing sequence information. It is well understood in the art that complementary sequences can be readily determined and/or analyzed, and that the description provided herein encompasses analytical methods performed in reference to a complementary sequence.

The section headings used herein are for organization purposes only and are not to be construed as limiting the subject matter described. The description is presented to enable one of ordinary skill in the art to make and use the invention and is provided in the context of a patent application and its requirements. Various modifications to the described embodiments will be readily apparent to those persons skilled in the art and the generic principles herein may be applied to other embodiments. Thus, the present invention is not intended to be limited to the embodiment shown but is to be accorded the widest scope consistent with the principles and features described herein.

The figures illustrate processes according to various embodiments. In the exemplary processes, some blocks are, optionally, combined, the order of some blocks is, optionally, changed, and some blocks are, optionally, omitted. In some examples, additional steps may be performed in combination with the exemplary processes. Accordingly, the operations as illustrated (and described in greater detail below) are exemplary by nature and, as such, should not be viewed as limiting.

The section headings used herein are for organizational purposes only and are not to be construed as limiting the subject matter described.

### Methods for Analyzing or Assigning a Disease Label

The disclosed methods employ statistical criteria for analyzing or assigning a disease label to a test sample. The disclosed methods comprise comparing the test sample against samples from a database, which have corresponding disease labels, according to quantitative criteria. The quantitative criteria can relate to genomic features of the test sample and/or the database samples. The quantitative criteria are used to determine a similarity score that assess the degree of similarity between the test sample and a database sample, e.g., the degree of genomic similarity between the test sample and the database sample. Similarity scores are determined between the test sample and multiple database samples, e.g., every database sample. The database samples can then be ranked according to their similarity scores, i.e., according to their similarities to the test sample. The database samples most similar to the test sample, i.e., the database samples with the highest similarity scores, can then be analyzed to determine the extent to which the test sample's disease label is represented amongst the most similar database samples, relative to the rest of the database samples. As part of this analysis, an enrichment score can be determined for the sample's disease label. The sample's disease label can be confirmed or rejected based on the analysis, e.g., based on the enrichment score. The sample's disease label can be a predetermined disease label, i.e., a disease label that already existed for the sample, prior to subjecting the sample to the method described herein. If rejecting the sample's predetermined disease label, a new disease label can be provided, e.g., an alternative diagnosis can be provided for the sample. If the sample does not already have a disease label, the method described herein can be used to provide a disease label. The method discussed herein ensures the consistent diagnosing of a test sample, by comparing the test sample against a database of samples. In doing so, clinical decisions based on the test sample's diagnosis are bettered, due to improved quality control.

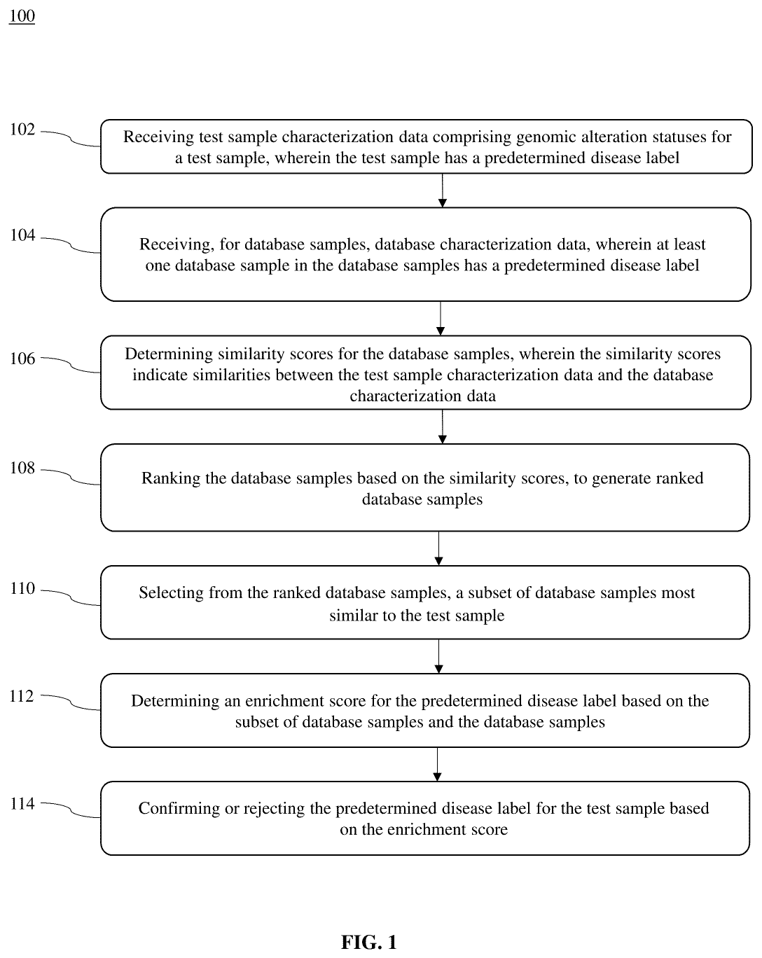

FIG. 1 provides an exemplary schematic for showing a general process 100 for analyzing or assigning a disease label to a sample. The method of analyzing or assigning a disease label to a sample can include: receiving test sample characterization data comprising genomic alteration statuses for a test sample, wherein the test sample has a predetermine disease label (102); receiving, for database samples, database characterization data, wherein at least one database sample in the database samples has a predetermined disease label (104); determining similarity scores for the database samples, wherein the similarity scores indicate similarities between the test sample characterization data and the database characterization data (106); ranking the database samples based on the similarity scores, to generate ranked database samples (108); selecting from the ranked database samples, a subset of database samples most similar to the test sample (110); determining an enrichment score for the predetermined disease label based on the subset of database samples and the database samples (112); and confirming or rejecting the predetermined disease label for the test sample based on the enrichment score (114).

Process 100 can be performed, for example, using one or more electronic devices implementing a software platform. In some examples, process 100 is performed using a client-server system, and the blocks of process 100 are divided up in any manner between the server and a client device. In other examples, the blocks of process 100 are divided up between the server and multiple client devices. Thus, while portions of process 100 are described herein as being performed by particular devices of a client-server system, it will be appreciated that process 100 is not so limited. In other examples, process 100 is performed using only a client device or only multiple client devices. In process 100, some blocks are, optionally, combined, the order of some blocks is, optionally, changed, and some blocks are, optionally, omitted. In some examples, additional steps may be performed in combination with the process 100. Accordingly, the operations as illustrated (and described in greater detail below) are exemplary by nature and, as such, should not be viewed as limiting.

In some instances, the analyzed or assigned disease label may be based on at least 1, at least 2, at least 3, at least 4, at least 5, at least 6, at least 7, at least 8, at least 9, at least 10, at least 20, at least 30, at least 40, or more than 40 genes. In some instances, the disclosed methods may be used to analyze or assign a disease label, by assessing the disease label of the test sample based on at least 1, at least 2, at least 3, at least 4, at least 5, at least 6, at least 7, at least 8, at least 9, at least 10, at least 20, at least 30, at least 40, or more than 40 gene loci. In some instances, the disclosed methods may be based on identified variants in the ABL1, ACVR1B, AKT1, AKT2, AKT3, ALK, ALOX12B, AMER1, APC, AR, ARAF, ARFRP1, ARID1A, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKN1A, CDKN1B, CDKN2A, CDKN2B, CDKN2C, CEBPA, CHEK1, CHEK2, CIC, CREBBP, CRKL, CSF1R, CSF3R, CTCF, CTNNA1, CTNNB1, CUL3, CUL4A, CXCR4, CYP17A1, DAXX, DDR1, DDR2, DIS3, DNMT3A, DOT1L, EED, EGFR, EMSY (C11orf30), EP300, EPHA3, EPHB1, EPHB4, ERBB2, ERBB3, ERBB4, ERCC4, ERG, ERRFI1, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNF1A, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MST1R, MTAP, MTOR, MUTYH, MYB, MYC, MYCL, MYCN, MYD88, NBN, NF1, NF2, NFE2L2, NFKBIA, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCD1LG2, PDGFRA, PDGFRB, PDK1, PIK3C2B, PIK3C2G, PIK3CA, PIK3CB, PIK3R1, PIM1, PMS2, POLD1, POLE, PPARG, PPP2R1A, PPP2R2A, PRDM1, PRKAR1A, PRKCI, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSC1L1, WT1, XPO1, XRCC2, ZNF217, or ZNF703 gene locus, or any combination thereof. In some instances, the disclosed methods may be based on identified variants in the ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CS1, CTLA-4, dMMR, EGFR, ERBB1, ERBB2, FGFR1-3, FLT3, GD2, HDAC, HER1, HER2, HR, IDH2, IL-1β, IL-6, IL-6R, JAK1, JAK2, JAK3, KIT, KRAS, MEK, MET, MSI-H, mTOR, PARP, PD-1, PDGFR, PDGFRα, PDGFRβ, PD-L1, PI3Kδ, PIGF, PTCH, RAF, RANKL, RET, ROS1, SLAMF7, VEGF, VEGFA, or VEGFB gene locus, or any combination thereof.

At 102 in FIG. 1, test sample characterization data comprising genomic alteration statuses for a test sample, wherein the test sample has a predetermined disease label, is received. The test sample characterization data can comprise non-genomic alteration statuses, such as, but not limited to, the race, ethnicity, sex, age, or disease history of the subject from which the test sample derives. The genomic alteration statuses for the test sample can comprise pathogenic genomic alteration statuses. The genomic alteration statuses for the test sample can comprise mutations, such as short variants, e.g., single nucleotide polymorphisms (SNPs), which can cause nonsense, missense, or frameshift mutations, as well as insertion-deletions (indels). The genomic alteration statuses can also comprise copy number level changes, such as copy number amplifications, or copy number deletions, as well as genomic rearrangements, such as arrangements comprising fusions and/or breaks between chromosomal segments, as well as genomic structures and/or sequences resulting from aneuploidy or chromothripsis. The genomic alteration statuses can also include mutational patterns or profiles, such as those catalogued in a database, e.g., the COSMIC database (Bamford et al. (2004), “The COSMIC (Catalogue of Somatic Mutations in Cancer) database and website”, British Journal of Cancer 91:355-358). The test sample can be a sample of interest or a sample under study, i.e., the sample being compared against the database of samples. The test sample can already have a corresponding disease label, e.g., the test sample can already have a corresponding diagnosis. The diagnosis can be made by one or more clinicians. The diagnosis can be made in accordance with a computational technique.

At 104 in FIG. 1, database characterization data is received, wherein at least one database sample in the database samples has a predetermined disease label. The at least one database characterization data can comprise non-genomic alteration statuses, such as, but not limited to, the race, ethnicity, sex, age, or disease history of the subject from which the test sample derives. The at least one database characterization data can comprise genomic alteration statuses. The genomic alteration statuses can include pathogenic genomic alteration statuses. The genomic alteration statuses of the database characterization data can include mutations, such as short variants, e.g., single nucleotide variant (SNVs), which can cause nonsense, missense, or frameshift mutations, as well as insertion-deletions (indels). The genomic alteration statuses can also comprise copy number level changes, such as copy number amplifications, or copy number deletions, as well as genomic rearrangements, such as arrangements comprising fusions and/or breaks between chromosomal segments, as well as genomic structures and/or sequences resulting from aneuploidy or chromothripsis. The genomic alteration statuses can also include mutational patterns or profiles, such as those catalogued in a database, e.g., the COSMIC database (Bamford et al. (2004), “The COSMIC (Catalogue of Somatic Mutations in Cancer) database and website”, British Journal of Cancer 91:355-358). The database sample can be a sample of interest or a sample under study, i.e., the sample being compared against the database of samples. The database of samples can be a database of cancer samples and their corresponding predetermined disease labels. A database sample can already have a corresponding disease label, e.g., a database sample can already have a corresponding diagnosis. The diagnosis or diagnoses of one or more database samples can be made by one or more clinicians. The diagnosis can be made in accordance with a computational technique.

At 106 in FIG. 1, similarity scores for the database samples are determined, wherein the similarity scores indicate similarities between the test sample characterization data and the database characterization data. The similarity score describing the similarity between the test sample and a database sample can be a real number, a rational number, an integer, a whole number, a natural number, or an irrational number. The similarity score between any two samples can be the summation of the total points awarded or penalized for each similarity, as described below.

The one or more of the determined similarity scores or one or more of the determined second similarity scores can increase by a predetermined pathogenic short variant scoring value, when the test sample and the corresponding database sample share a pathogenic short variant affecting a same gene. A pathogenic short variant affecting the same gene for both the test sample and a database sample can result in the addition of 10 to the similarity score, i.e., the predetermined pathogenic short variant scoring value can be 10.

The one or more of the determined similarity scores or one or more of the determined second similarity scores can increase by a predetermined same pathogenic effect scoring value, when the test sample and the corresponding database sample share a pathogenic short variant affecting a same gene with identical protein effects. A pathogenic short variant affecting the same gene with identical protein effects for both the test sample and a database sample can result in the addition of 5 to the similarity score, i.e., the predetermined same pathogenic effect scoring value can be 5.

The one or more of the determined similarity scores or one or more of the determined second similarity scores can increase by a predetermined pathogenic copy number amplification scoring value, when the test sample and the corresponding database sample share a pathogenic copy number amplification occurring on a same amplicon segment. The one or more of the determined similarity scores or one or more of the determined second similarity scores can increase by a predetermined pathogenic copy number deletion scoring value, when the test sample and the corresponding database sample share a pathogenic copy number deletion occurring on a same commonly deleted segment. Genes that are present on the same chromosome and are classified as being co-amplified along the amplicon segment or co-deleted along the commonly deleted segment, are identified, for both the test sample and a database sample. Of those co-amplified or co-deleted genes, genes exhibiting an odds ratio of 50 or greater relative to the samples in the database can be selected. The odds ratio is determined by a two-by-two contingency table, where one axis of the contingency table consists of whether one of two genes is amplified or not, in the case of co-amplification, and the other axis of the contingency table consists of whether the other one of the two genes is amplified or not, in the case of co-amplification. Similarly, for co-deletion, the odds ratio is determined by a two-by-two contingency table where one axis of the contingency table consists of whether one of two genes is deleted or not, and the other axis of the contingency table consist of whether the other one of the two genes is deleted or not. If, as indicated by the odds ratio being greater than, for example, 50, the two genes can be classified as being co-amplified or co-deleted. The similarity scores can then be determined based on whether the test sample and a database sample possess a common co-amplified or co-deleted segment, of which the selected genes are found. A pathogenic copy number amplification occurring on the same amplicon segment for both the test sample and a database sample can result in the addition of 5 to the similarity score, i.e., the predetermined pathogenic copy number amplification scoring value can be 5. A pathogenic copy number deletion occurring on the same commonly deleted segment can result in the addition of 5 to the similarity score, i.e., the predetermined pathogenic copy number deletion scoring value can be 5.

The one or more of the determined similarity scores or one or more of the determined second similarity scores can increase by a predetermined pathogenic rearrangement scoring value, when the test sample and the corresponding database sample share a same two gene partners in a pathogenic rearrangement. A pathogenic rearrangement can result in the addition of 15 to the similarity score, if the same two gene partners are affected in both the test sample and a database sample, i.e., the predetermined pathogenic rearrangement scoring value can be 15. The one or more of the determined similarity scores or one or more of the determined second similarity scores can increase by a predetermined same gene partner pathogenic rearrangement scoring value, when the test sample and the corresponding database sample share a same one gene partner in a pathogenic rearrangement. A pathogenic rearrangement can result in the addition of 7.5 to the similarity score, if only one gene partner is shared between the test sample and a database sample, i.e., the predetermined same gene partner pathogenic rearrangement scoring value can be 7.5. Rearrangements affecting only a single gene that are common to both the test sample and a database sample can result in the addition of 7.5 to the similarity score, i.e., the predetermined same gene partner pathogenic rearrangement scoring value can be 7.5.

The one or more of the determined similarity scores or one or more of the determined second similarity scores can decrease by a predetermined non-common genomic alteration status scoring value, when the test sample and the corresponding database sample do not share a same genomic alteration status from the genomic alteration statuses. If a gene alteration in both the test sample and a database sample do not match, a mismatch penalty of −1 can be added to the similarity score, i.e., the similarity score can decrease by 1, i.e., the predetermined non-common genomic alteration status scoring value can be −1.

The one or more of the determined similarity scores or one or more of the determined second similarity scores can increase by a predetermined tumor mutational burden (TMB) scoring value, when the test sample and the corresponding database sample each have a TMB score above a predetermined TMB score threshold. If both the test sample and a database sample are determined to have a high TMB status, e.g., if the sample has 10 mutations per megabase or greater, the similarity score can increase by 10, i.e., the predetermined TMB scoring value can be 10. The one or more of the determined similarity scores or one or more of the determined second similarity scores can increase by a predetermined dominant mutational signature scoring value, when the test sample and the one database sample share a dominant mutational signature. The dominant mutational signature can be associated with exposure to an alkylating agent, tobacco, or ultraviolet light, or an altered activity of APOBEC, a mutation in one or more mismatch repair pathway genes, or a mutation in a POLE gene, or a mutation in BRCA1 or BRCA2. If both the test sample and a database sample share a dominant mutational signature, the similarity score can be increased by 10, i.e., the predetermined dominant mutational signature scoring value can be 10. The one or more of the determined similarity scores or one or more of the determined second similarity scores can increase by a predetermined high TMB and dominant mutational signature scoring value, when the test sample and the corresponding database sample share both the high TMB score and the dominant mutational signature. That is, if both the test sample and a database sample both have a high TMB status and have a common dominant mutational signature, the similarity score can increase by at most 15, i.e., the sum of the predetermined TMB scoring value and the predetermined dominant mutational signature scoring value can be at most 15, i.e., the predetermined high TMB and dominant mutational signature scoring value can be 15.

The one or more determined similarity scores or the one or more determined second similarity scores can increase by a predetermined copy number signature scoring value, when the test sample and the corresponding database sample share a copy number signature. The copy number signature can refer to copy number alterations, e.g., copy number amplifications or copy number deletions, where the genomic sequences for which the copy numbers are altered, correspond to a biological process, such as a mutational process. For example, copy number alterations comprising the BRCA1 and/or BRCA2 genes can be related to, and can be caused by or can cause, homologous recombination deficiency (HRD). Similarly, copy number alterations of other genes can be related to other biological processes and/or etiologies, and such copy number alterations and their corresponding altered biological process can be referred to as a copy number signature. Other example copy number signatures can include signatures related to genome-wide loss of heterozygosity, HRD under gynecological contexts, HRD under breast tissue contexts, HRD under prostate contexts, chromosomal instability, focal tandem duplications, seismic amplifications, DNA mismatch repair, high microsatellite instability, oscillating copy number states, neuroendocrine conditions, and/or subclonal conditions, e.g., conditions comprising the presence of sublpoidy changepoints centered around a predetermined number of copies for a diploid sample. The copy number signature can include copy number signatures as described in Moore et al., (2023) JCO Precision Oncology. 7(e2300093). If both the test sample and a database sample share a copy number signature, the similarity score can be increased by 10, i.e., the predetermined copy number signature scoring value can be 10.

The one or more determined similarity scores or the one or more determined second similarity scores can increase by a predetermined aneuploidy feature scoring value, when the test sample and the corresponding database sample share a common aneuploidy feature. The common aneuploidy features can include features identified by analyzing arm-level or cytoband-level events, e.g., if samples share a chromosomal 19p loss or a chromosomal 19q gain, the predetermined aneuploidy feature scoring value can change. In addition, the common aneuploidy features can be a combination of aneuploidy burden (e.g., the number of chromosome arms with aneuploidy) and based on the presence and/or absence of specific cytogenetic events (e.g., loss or gain of specific chromosome arms or specific cytobands, etc.). Aneuploidy features can include features described in Sharaf et al., (2021) Neuro-Oncology Advances. 3(1): vdab017. If both the test sample and a database sample share a common aneuploidy feature, the similarity score can be increased by 10, i.e., the common aneuploidy feature scoring value can be 10.

At 108 in FIG. 1, the database samples are ranked based on the similarity scores, to generate ranked database samples. The ranks based on the similarity scores can be ordered according to ascending rank or descending rank. The ranking can be used to determine the subset of database samples most similar to the test sample. The subset of database samples most similar to the test sample can be determined by eliminating the database samples that are least similar to the test sample, and/or by including the database samples that are most similar to the test sample. Numerically, the subset of database samples most similar to the test sample can be determined by eliminating the database samples with the lowest similarity scores, and/or by including the database samples with the highest similarity scores.

At 110 in FIG. 1, from the ranked database samples, a subset of database samples most similar to the test sample are selected. All the database samples from the ranked database samples can have the determined similarity score or the determined second similarity score of at least a minimum score. A minimum score can be a score of 10, i.e., a similarity score between the test sample and a database sample can be 10. Further thresholding can be done on the on the ranked database samples. For example, the number of samples in the subset of database samples can be limited, e.g., to the top 800 ranked samples. Multiple thresholds can be applied on the ranked database samples, e.g., the subset of database samples most similar to the test sample can include exclusively the top 800 ranked samples and the top 800 samples can each comprise a minimum score of 10, i.e., a minimum similarity score of 10.

At 112 in FIG. 1, an enrichment score is determined for the predetermined disease label based on the subset of database samples and the database samples. The enrichment score can be a metric that describes the extent to which the predetermined disease label is represented in the subset of database samples, e.g., the database samples most similar to the test sample, relative to a superset of the subset of database samples, e.g., the remaining database samples or the entirety of the database samples. The database samples corresponding to a predetermined number of most similar databases samples can be used for determining the enrichment score or the second enrichment score, i.e., the enrichment score or the second enrichment score can be computed for the database samples above a minimum similarity score. The enrichment score or the second enrichment score can be an odds ratio. For example, the odds ratio can be computed by ascertaining the following four values: a) the number of samples of the same predetermined disease label as the test sample, amongst the highest scoring database samples; b) the number of samples different from the same predetermined disease label as the test sample, amongst the highest scoring database samples; c) the number of samples of the same predetermined disease label as the test sample, amongst all the database samples; and d) the number of samples different from the same predetermined disease label as the test sample, amongst all the database samples. The odds ratio can then be determined by dividing a) by b), to get a normalized number of samples with the same predetermined disease label as the test sample, which can then be divided by the result of dividing c) by d), i.e., the normalized number of samples without the same predetermined disease label as the test sample. The odds ratio can be determined by Fisher's exact test. The enrichment score or the second enrichment score can be a U score from a Mann Whitney U-test. The enrichment score or the second enrichment score can be other statistical results and/or the result of other statistical methods.

The methods disclosed herein can further comprise determining a confidence value indicating whether the predetermined disease label is correctly confirmed or rejected. In general, the confidence value can refer to any kind of value that quantifies the degree of confidence and/or believability of data, such as, for example, a p-value. Probability values regarding the data, such as the likelihood of observing an event, such as the data acquired from an experiment, can be confidence values. Confidence intervals can also be confidence values. The effect size of results seen in data can also be confidence values. The confidence value can be a p-value determined by the Fisher's exact test. The p-value determined by the Fisher's exact test can be based on the four values from which an odds ratio for the predetermined disease label can be determined: a) the number of samples of the same predetermined disease label as the test sample, amongst the highest scoring database samples; b) the number of samples different from the same predetermined disease label as the test sample, amongst the highest scoring database samples; c) the number of samples of the same predetermined disease label as the test sample, amongst all database samples not included in the highest scoring database samples; and d) the number of samples different from the same predetermined disease label as the test sample, amongst all database samples not included in the highest scoring database samples. More specifically, the p-value can be determined according to the following formula, for which the terms a, b, c, and d correspond to the descriptions of a) b) c) and d) described within this paragraph, and n is the total number of samples:

$$\frac{( {c}_{a}^{a + c} ) ( {c}_{b}^{b + d} )}{{c}_{a + b}^{n}}$$

The p-value can describe the statistical significance of the observed enrichment score or second enrichment score, e.g., odds ratio, for the predetermined disease label associated with the test sample.

At 114 in FIG. 1, the predetermined disease label for the test sample is confirmed or rejected, based on the enrichment score. The predetermined disease label can be rejected, when the enrichment score for the predetermined disease label is less than or equal to a first predetermined enrichment score threshold. For example, the predetermined disease label, e.g., diagnosis associated with the test sample, can be rejected, if the enrichment score for the predetermined disease label is less than or equal to a first predetermined enrichment score threshold, e.g., threshold of 0.5. An enrichment score, e.g., odds ratio of less than 1 can suggest that the predetermined disease label is depleted amongst the subset of database samples, e.g., the database samples most similar to the test sample. The predetermined disease label can be rejected, when the confidence value can be less than or equal to a first predetermined confidence value threshold. For example, the predetermined disease label, e.g., diagnosis associated with the test sample, can be rejected, if the confidence value is less than or equal to a first predetermined confidence value threshold, e.g., threshold of 10⁻⁵. A small confidence value can suggest that the likelihood that the observed enrichment score is due to random chance alone, is approximately equivalent to the small confidence value, e.g., p-value. The predetermined disease label can be rejected, when the enrichment score for the predetermined disease label is less than or equal to the first predetermined enrichment score threshold and the confidence value is less than or equal to the first predetermined confidence value threshold. For example, the predetermined disease label for the test sample can be rejected if both the odds ratio of the predetermined disease label is 0.5 or less, and the p-value of the predetermined disease label is 10⁻⁵ or less. The predetermined disease label can refer to a diagnosis associated with the test sample. The predetermined disease label can refer to varying levels of diagnostic granularity. For example, the predetermined disease label can refer to a disease group, e.g., a general group of cancers, such as non-small cell lung cancer The predetermined disease label can also refer to a disease ontology, which can include more specific disease types than those identified at the level of a disease group, e.g., lung adenocarcinoma. Rejecting the predetermined disease label associated with the test sample can be based on both the disease group and the disease ontology of the predetermined disease label having an enrichment score less than a first predetermined enrichment score threshold, and the disease group and/or the disease ontology of the predetermined disease label having a confidence value less than a first predetermined confidence value threshold. For example, the predetermined disease label associated with the test sample, e.g., the original diagnosis associated with the test sample, can be rejected if the odds ratio of the disease group of the test sample is 0.5 or less, with a p-value of 10⁻⁵ or less, and if the odds ratio of the disease ontology of the predetermined disease label is 0.5 or less, and a p-value threshold need not be provided for the disease ontology.

In some aspects, the methods disclosed herein can further comprise: determining, by the one or more processors, one or more alternate enrichment scores for one or more alternate disease labels based on the subset of database samples and the database samples; and rejecting, by the one or more processors, the predetermined disease label and assigning an alternate disease label for the test sample based on the enrichment score. That is, in the case that the predetermined disease label for the test sample is rejected, a different, i.e., alternate, disease label, e.g., diagnosis, can be assigned to the test sample. The alternate disease label can be accepted, when the enrichment score for the alternate disease label is greater than or equal to a second predetermined enrichment score threshold. For example, the alternate disease label, e.g., new potential diagnosis for the test sample, can be accepted, if the enrichment score for the alternate disease label is greater than or equal to a second predetermined enrichment score threshold, e.g., threshold of 2.0. An enrichment score, e.g., odds ratio of greater than 1 can suggest that the predetermined disease label is enriched amongst the subset of database samples, e.g., the database samples most similar to the test sample. The alternate disease label can be accepted, when the confidence value is less than or equal to a second predetermined confidence value threshold. For example, the predetermined disease label, e.g., new potential diagnosis for the test sample, can be accepted, if the confidence value is less than or equal to a second predetermined confidence value threshold, e.g., threshold of 10⁻⁵. A small confidence value can suggest that the likelihood that the observed enrichment score is due to random chance alone, is approximately equivalent to the small confidence value, e.g., p-value. The alternate disease label can be accepted, when the enrichment score for the predetermined disease label is greater than or equal to the second predetermined enrichment score threshold and the confidence value is less than or equal to the second predetermined confidence value threshold. For example, the alternate disease label for the test sample can be accepted if both the odds ratio of the alternate disease label is 2.0 or greater, and the p-value of the predetermined disease label is 10⁻⁵ or less. The alternate disease label can refer to a new potential diagnosis associated with the test sample. The alternate disease label can refer to varying levels of diagnostic granularity. For example, the alternate disease label can refer to a disease group, e.g., a general group of cancers, such as non-small cell lung cancer. The alternate disease label can also refer to a disease ontology, which can include more specific disease types than those identified at the level of a disease group, e.g., lung adenocarcinoma. Accepting the alternate disease label for the test sample can be based on both the disease group and the disease ontology of the alternate disease label having an enrichment score greater than a second predetermined enrichments core threshold, and the disease group and/or the disease ontology of the alternate disease label having a confidence value less than a second predetermined confidence value threshold. For example, the alternate disease label for the test sample, e.g., the potential new diagnosis for the test sample, can be accepted if the odds ratio of the disease group of the test sample is 2.0 or greater, with a p-value of 10⁻⁵ or less, and if the odds ratio of the disease ontology of the alternate disease label is 2.0 or greater, with a p-value of 10⁻⁵ or less. The alternate disease label can be a cancer. The alternate disease label can indicate that the disease is unknown. The disease can be unknown when the number of database samples in the ranked database samples is less than or equal to a predetermined number of database samples. The unknown disease can refer to a disease label that does not specify a specific disease for the test sample, because, for example, of insufficient evidence. Insufficient evidence can refer to the number of database samples in the ranked database samples having a similarity score of at least some threshold similarity score, e.g., 10, being less than the predetermined number of database samples, e.g., 300. That is, for example, if less than 300 database samples have a similarity score greater than 10, the test sample can be assigned a disease where the disease is unknown, i.e., no disease type, e.g., disease group or disease ontology, is assigned to the sample. The alternate disease label can be a cancer of unknown primary. A cancer of unknown primary can include cancers where the malignant, e.g., cancer cells are found in the body of the subject, but the place the cancer began is not known. For example, metastasized cells are identified in the body of the subject, but the primary source of the metastasized cells are unknown.

In some aspects, the methods disclosed herein can further comprise: excluding, by the one or more processors, for the test sample, a genomic alteration status from the test sample characterization data, to generate reduced test sample characterization data; excluding, by the one or more processors, for the database samples, the genomic alteration status from the database characterization data, to generate reduced database characterization data; determining, by the one or more processors, second similarity scores, wherein the second similarity scores indicate similarities between the reduced test sample characterization data, and for a corresponding database sample, the reduced database characterization data; ranking, by the one or more processors, the database samples based on the second similarity scores, to generate second ranked database samples; selecting, by the one or more processors, from the second ranked database samples, a second subset of database samples most similar to the test sample; determining, by the one or more processors, a second enrichment score for the predetermined disease label based on the second subset of database samples and the database samples; and confirming or rejecting, by the one or more processors, the predetermined disease label for the test sample based on the enrichment score and the second enrichment score. For example, a set of similarity scores can still be determined between the test sample and the database samples, where each similarity score in the set of similarity scores corresponds to a comparison between the test sample and a database sample. In addition, however, each comparison between the test sample and a database sample can result in another set of similarity scores, because for each test sample and database sample comparison, a genomic alteration status can be removed, after which a similarity score can again be determined. The removal of a genomic alteration status before computing another similarity score can be iterated, for example, for each genomic alteration status in the genomic alteration statuses of the test sample characterization data and a database sample characterization data. The result of such a perturbation-based approach to the genomic alteration statuses is a) a set of similarity scores between the test sample and the database samples, of length m, where m can be the number of database samples or less, and b) m sets of similarity scores between the test sample and a database sample, where each set of m sets can be of length n, where n can be the number of genomic alteration statuses minus one being compared between the test sample and a database sample. If, for example, n is approximately similar for all m number of database samples, then the total number of similarity scores being analyzed can be approximately m×n. Expressed another way, if n is the highest observed number of genomic alteration statuses when comparing the test sample against a database sample, the maximum number of similarity scores being analyzed for a test sample can be m×n. The process of systematically excluding one genomic alteration status at a time from the test sample characterization data and the database characterization data, and then determining the similarity scores, is to prevent the analyses from confirming or rejecting a predetermined disease label and/or accepting an alternate disease label based on the disproportionate contribution of a single genomic alteration status. That is, the methods described herein aim to confirm or reject a predetermined disease label and/or accept an alternate disease label based on multiple lines of evidence, i.e., based on multiple features of the test sample characterization data and the database characterization data, e.g., based on multiple genomic alteration statuses based on the test sample characterization data and the database characterization data. In doing so, the methods described herein provide a robust and high confidence basis for confirming or rejecting a predetermined disease label and/or accepting an alternate disease label. The excluding of one genomic alteration status, and then determining again another similarity score between the test sample and a database sample, can be referred to as applying a leave-one-out (LOO) contingency filter.

All the database samples from the ranked database samples can have the determined similarity score or the determined second similarity score of at least a minimum score. The minimum score can be 10. If an insufficient number of the ranked database samples possess a similarity score of 10 or higher, where an insufficient number is a number less than a predetermined threshold number, such as 300, then the test sample can be assigned an alternate disease label, where the disease is unknown.

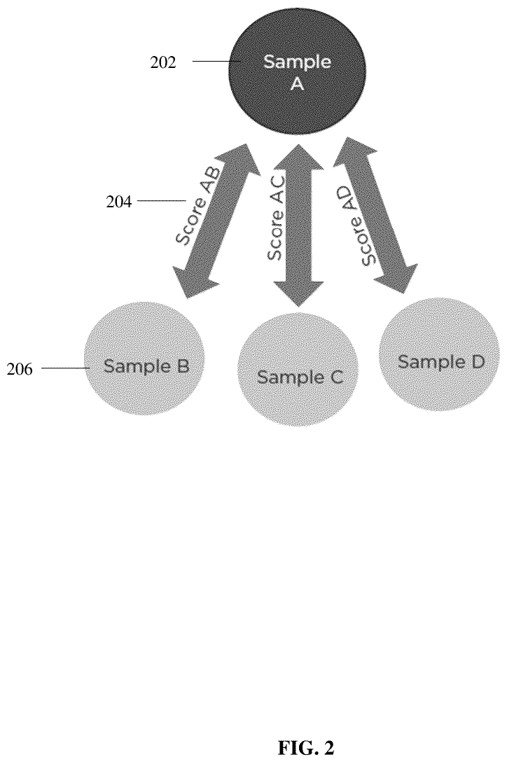

FIG. 2 depicts a schematic illustrating a method by which a test sample A 202 can be compared against some database samples, such as database sample B 206, database sample C, and database sample C. The test sample A 202 is compared against a database sample, such as database sample B 206, to generate a similarity score comparing the test sample and the database sample, such as score AB 204, which compares sample A 202 to sample B 206. Similarly, sample A is compared against sample C to generate a similarity score AC, and sample A is compared against sample D to generate a similarity score AD. The test sample A can be compared against many database samples, such as database samples in addition samples B, C, and D depicted in FIG. 2. The test sample A can be associated to a diagnosed disease, such as a cancer.

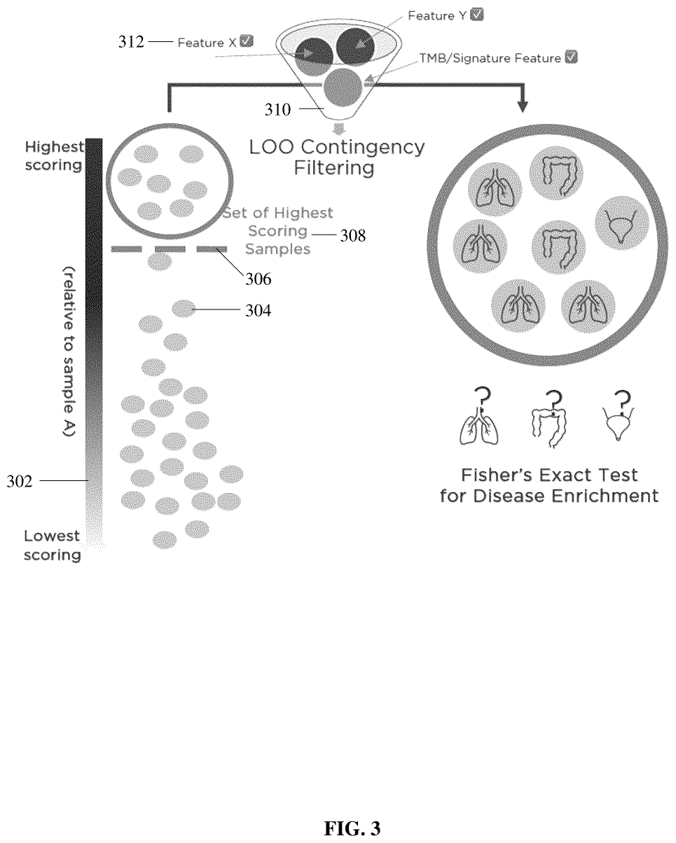

FIG. 3 extends the concept exemplified in FIG. 2, such that multiple database samples, are compared relative to test sample 202, to generate a similarity score between the test sample A and a given database sample. The database samples are then ranked according to their similarity score, i.e., the score that quantifies the extent to which a given database sample is similar to test sample A, as depicted on scale 302. Database samples with similarity scores that are lower than a predetermined threshold 306, such as database sample 304, are eliminated from further analyses, because those database samples are not similar enough to test sample A. The remaining database samples, all of which possess similarity scores greater than the predetermined threshold 306, constitute the set of highest similarity score scoring samples 308. The set of highest similarity score scoring samples 308 are the only samples used for downstream analyses.

FIG. 3 depicts a leave-one-out (LOO) contingency filter 310, which comprises recalculating pairwise similarity scores for one or more database samples versus the test sample A, but with a single given feature, such as feature X 312, left out, when determining the pairwise similarity scores. The LOO contingency filter 310 prevents any given similarity score from being overwhelmingly determined by the contribution of a single feature, such as feature X 312. The LOO contingency filter 310 can be applied for different features, one feature at a time, and not just feature X 312. For example, the LOO contingency filter can be run between the test sample A and the database samples, such that feature Y is not considered, during the comparison and determining of the similarity scores. Each LOO contingency filter run results in a set of similarity scores, and each of those similarity scores can be subjected to a predetermined threshold, like the predetermined threshold 308. Each set of threshold-passing similarity scores are then used to compute an enrichment score for a diagnosis of interest, such as an odds ratio, which describes the extent to which the diagnosis of interest, such as the original diagnosis associated with the sample (if the original diagnosis exists), is represented in the database. The sets of threshold-passing similarity scores can also be used to compute a confidence value, such as a p-value from Fisher's exact test. The set of threshold-passing similarity scores for which the LOO contingency filter 310 is not applied, can also be used to compute an enrichment score and a confidence value. If the enrichment score is less than some predetermined threshold, such as an odds ratio of 0.5, and if the confidence value is less than some other predetermined threshold, such as a p-value of 10⁻⁵, then the original diagnosis associated with test sample A, if the original diagnosis existed, can be rejected. Otherwise, the diagnosis associated with test sample A can be not rejected. If the original diagnosis associated with the sample is rejected, an alternative diagnosis can be made for the sample, provided that an enrichment score for the alternative diagnosis in the set of similarity scores is greater than some predetermined threshold, and the confidence value for the alternative diagnosis in the set of similarity scores is less than some other predetermined value. The sets of threshold-passing similarity scores for which the LOO contingency filter 310 is applied, as well as the resulting enrichment scores and confidence values, can be considered together with the set of threshold-passing similarity scores for which the LOO contingency filter 310 is not applied, as well as the resulting enrichment score and confidence value. For example, the highest similarity score for the predetermined disease label of the sample, and the single lowest similarity score from the sets of threshold-passing similarity scores for which the LOO contingency filter 310 is applied, can be examined. If the lowest similarity score resulting from applying the LOO contingency filter 310 is greater than a predetermined threshold value, the disease label corresponding to the lowest similarity score can be used as a possible disease label, which can result in keeping the predetermined disease label as is, or adopting an alternative disease label.

### Methods of Use of an Assigned Disease Label

In some instances, the disclosed methods may further comprise one or more of the steps of: (i) obtaining the sample from the subject (e.g., a subject suspected of having or determined to have cancer), (ii) extracting nucleic acid molecules (e.g., a mixture of tumor nucleic acid molecules and non-tumor nucleic acid molecules) from the sample, (iii) ligating one or more adapters to the nucleic acid molecules extracted from the sample (e.g., one or more amplification primers, flow cell adaptor sequences, substrate adapter sequences, or sample index sequences), (iv) performing a methylation conversion reaction to convert, e.g., non-methylated cytosine to uracil, (v) amplifying the nucleic acid molecules (e.g., using a polymerase chain reaction (PCR) amplification technique, a non-PCR amplification technique, or an isothermal amplification technique), (vi) capturing nucleic acid molecules from the amplified nucleic acid molecules (e.g., by hybridization to one or more bait molecules, where the bait molecules each comprise one or more nucleic acid molecules that each comprising a region that is complementary to a region of a captured nucleic acid molecule), (vii) sequencing the nucleic acid molecules extracted from the sample (or library proxies derived therefrom) using, e.g., a next-generation (massively parallel) sequencing technique, a whole genome sequencing (WGS) technique, a whole exome sequencing technique, a targeted sequencing technique, a direct sequencing technique, or a Sanger sequencing technique) using, e.g., a next-generation (massively parallel) sequencer, and (viii) generating, displaying, transmitting, and/or delivering a report (e.g., an electronic, web-based, or paper report) to the subject (or patient), a caregiver, a healthcare provider, a physician, an oncologist, an electronic medical record system, a hospital, a clinic, a third-party payer, an insurance company, or a government office. In some instances, the report comprises output from the methods described herein. In some instances, all or a portion of the report may be displayed in the graphical user interface of an online or web-based healthcare portal. In some instances, the report is transmitted via a computer network or peer-to-peer connection.

The disclosed methods may be used with any of a variety of samples. For example, in some instances, the sample may comprise a tissue biopsy sample, a liquid biopsy sample, or a normal control. In some instances, the sample may be a liquid biopsy sample and may comprise blood, plasma, cerebrospinal fluid, sputum, stool, urine, or saliva. In some instances, the sample may be a liquid biopsy sample and may comprise circulating tumor cells (CTCs). In some instances, the sample may be a liquid biopsy sample and may comprise cell-free DNA (cfDNA), circulating tumor DNA (ctDNA), or any combination thereof.

In some instances, the nucleic acid molecules extracted from a sample may comprise a mixture of tumor nucleic acid molecules and non-tumor nucleic acid molecules. In some instances, the tumor nucleic acid molecules may be derived from a tumor portion of a heterogeneous tissue biopsy sample, and the non-tumor nucleic acid molecules may be derived from a normal portion of the heterogeneous tissue biopsy sample. In some instances, the sample may comprise a liquid biopsy sample, and the tumor nucleic acid molecules may be derived from a circulating tumor DNA (ctDNA) fraction of the liquid biopsy sample while the non-tumor nucleic acid molecules may be derived from a non-tumor, cell-free DNA (cfDNA) fraction of the liquid biopsy sample.

In some instances, the disclosed methods for analyzing or assigning a disease label may be used to diagnose (or as part of a diagnosis of) the presence of disease or other condition (e.g., cancer, genetic disorders (such as Down Syndrome and Fragile X), neurological disorders, or any other disease type where detection of variants, e.g., copy number alternations, are relevant to diagnosing, treating, or predicting said disease) in a subject (e.g., a patient). In some instances, the disclosed methods may be applicable to diagnosis of any of a variety of cancers as described elsewhere herein.

In some instances, the disclosed methods for analyzing or assigning a disease label may be used to predict genetic disorders in fetal DNA. (e.g., for invasive or non-invasive prenatal testing). For example, sequence read data obtained by sequencing fetal DNA extracted from samples obtained using invasive amniocentesis, chorionic villus sampling (cVS), or fetal umbilical cord sampling techniques, or obtained using non-invasive sampling of cell-free DNA (cfDNA) samples (which comprises a mix of maternal cfDNA and fetal cfDNA), may be processed according to the disclosed methods to identify variants, e.g., copy number alterations, associated with, e.g., Down Syndrome (trisomy 21), trisomy 18, trisomy 13, and extra or missing copies of the X and Y chromosomes.

In some instances, the disclosed methods for analyzing or assigning a disease label may be used to select a subject (e.g., a patient) for a clinical trial based on the disease label determined based on one or more genomic alteration statuses. In some instances, patient selection for clinical trials based on, e.g., analyzing or assigning a disease label, may accelerate the development of targeted therapies and improve the healthcare outcomes for treatment decisions.

In some instances, the disclosed methods for analyzing or assigning a disease label may be used to select an appropriate therapy or treatment (e.g., an anti-cancer therapy or anti-cancer treatment) for a subject. In some instances, for example, the anti-cancer therapy or treatment may comprise use of a poly (ADP-ribose) polymerase inhibitor (PARPi), a platinum compound, chemotherapy, radiation therapy, a targeted therapy (e.g., immunotherapy), surgery, or any combination thereof.

In some instances, the targeted therapy (or anti-cancer target therapy) may comprise abemaciclib (Verzenio), abiraterone acetate (Zytiga), acalabrutinib (Calquence), ado-trastuzumab emtansine (Kadcyla), afatinib dimaleate (Gilotrif), aldesleukin (Proleukin), alectinib (Alecensa), alemtuzumab (Campath), alitretinoin (Panretin), alpelisib (Piqray), amivantamab-vmjw (Rybrevant), anastrozole (Arimidex), apalutamide (Erleada), asciminib hydrochloride (Scemblix), atezolizumab (Tecentriq), avapritinib (Ayvakit), avelumab (Bavencio), axicabtagene ciloleucel (Yescarta), axitinib (Inlyta), belantamab mafodotin-blmf (Blenrep), belimumab (Benlysta), belinostat (Beleodaq), belzutifan (Welireg), bevacizumab (Avastin), bexarotene (Targretin), binimetinib (Mektovi), blinatumomab (Blincyto), bortezomib (Velcade), bosutinib (Bosulif), brentuximab vedotin (Adcetris), brexucabtagene autoleucel (Tecartus), brigatinib (Alunbrig), cabazitaxel (Jevtana), cabozantinib (Cabometyx), cabozantinib (Cabometyx, Cometriq), canakinumab (Ilaris), capmatinib hydrochloride (Tabrecta), capivasertib, carfilzomib (Kyprolis), cemiplimab-rwlc (Libtayo), ceritinib (LDK378/Zykadia), cetuximab (Erbitux), cobimetinib (Cotellic), copanlisib hydrochloride (Aligopa), crizotinib (Xalkori), dabrafenib (Tafinlar), dacomitinib (Vizimpro), daratumumab (Darzalex), daratumumab and hyaluronidase-fihj (Darzalex Faspro), darolutamide (Nubega), dasatinib (Sprycel), denileukin diftitox (Ontak), denosumab (Xgeva), dinutuximab (Unituxin), dostarlimab-gxly (Jemperli), durvalumab (Imfinzi), duvelisib (Copiktra), elotuzumab (Empliciti), enasidenib mesylate (Idhifa), encorafenib (Braftovi), enfortumab vedotin-ejfv (Padcev), entrectinib (Rozlytrek), enzalutamide (Xtandi), erdafitinib (Balversa), erlotinib (Tarceva), everolimus (Afinitor), exemestane (Aromasin), fam-trastuzumab deruxtecan-nxki (Enhertu), fedratinib hydrochloride (Inrebic), fulvestrant (Faslodex), gefitinib (Iressa), gemtuzumab ozogamicin (Mylotarg), gilteritinib (Xospata), glasdegib maleate (Daurismo), hyaluronidase-zzxf (Phesgo), ibrutinib (Imbruvica), ibritumomab tiuxetan (Zevalin), idecabtagene vicleucel (Abecma), idelalisib (Zydelig), imatinib mesylate (Gleevec), infigratinib phosphate (Truseltiq), inotuzumab ozogamicin (Besponsa), iobenguane I131 (Azedra), ipilimumab (Yervoy), isatuximab-irfc (Sarclisa), ivosidenib (Tibsovo), ixazomib citrate (Ninlaro), lanreotide acetate (Somatuline Depot), lapatinib (Tykerb), larotrectinib sulfate (Vitrakvi), lenvatinib mesylate (Lenvima), letrozole (Femara), lisocabtagene maraleucel (Breyanzi), loncastuximab tesirine-lpyl (Zynlonta), lorlatinib (Lorbrena), lutetium Lu 177-dotatate (Lutathera), margetuximab-cmkb (Margenza), midostaurin (Rydapt), mobocertinib succinate (Exkivity), mogamulizumab-kpkc (Poteligeo), moxetumomab pasudotox-tdfk (Lumoxiti), naxitamab-gqgk (Danyelza), necitumumab (Portrazza), neratinib maleate (Nerlynx), nilotinib (Tasigna), niraparib tosylate monohydrate (Zejula), nivolumab (Opdivo), obinutuzumab (Gazyva), ofatumumab (Arzerra), olaparib (Lynparza), olaratumab (Lartruvo), osimertinib (Tagrisso), palbociclib (Ibrance), panitumumab (Vectibix), panobinostat (Farydak), pazopanib (Votrient), pembrolizumab (Keytruda), pemigatinib (Pemazyre), pertuzumab (Perjeta), pexidartinib hydrochloride (Turalio), polatuzumab vedotin-piiq (Polivy), ponatinib hydrochloride (Iclusig), pralatrexate (Folotyn), pralsetinib (Gavreto), radium 223 dichloride (Xofigo), ramucirumab (Cyramza), regorafenib (Stivarga), ribociclib (Kisqali), ripretinib (Qinlock), rituximab (Rituxan), rituximab and hyaluronidase human (Rituxan Hycela), romidepsin (Istodax), rucaparib camsylate (Rubraca), ruxolitinib phosphate (Jakafi), sacituzumab govitecan-hziy (Trodelvy), seliciclib, selinexor (Xpovio), selpercatinib (Retevmo), selumetinib sulfate (Koselugo), siltuximab (Sylvant), sipuleucel-T (Provenge), sirolimus protein-bound particles (Fyarro), sonidegib (Odomzo), sorafenib (Nexavar), sotorasib (Lumakras), sunitinib (Sutent), tafasitamab-cxix (Monjuvi), tagraxofusp-erzs (Elzonris), talazoparib tosylate (Talzenna), tamoxifen (Nolvadex), tazemetostat hydrobromide (Tazverik), tebentafusp-tebn (Kimmtrak), temsirolimus (Torisel), tepotinib hydrochloride (Tepmetko), tisagenlecleucel (Kymriah), tisotumab vedotin-tftv (Tivdak), tocilizumab (Actemra), tofacitinib (Xeljanz), tositumomab (Bexxar), trametinib (Mekinist), trastuzumab (Herceptin), tretinoin (Vesanoid), tivozanib hydrochloride (Fotivda), toremifene (Fareston), tucatinib (Tukysa), umbralisib tosylate (Ukoniq), vandetanib (Caprelsa), vemurafenib (Zelboraf), venetoclax (Venclexta), vismodegib (Erivedge), vorinostat (Zolinza), zanubrutinib (Brukinsa), ziv-aflibercept (Zaltrap), or any combination thereof.

In some instances, the disclosed methods for analyzing or assigning a disease label may be used in treating a disease (e.g., a cancer) in a subject. For example, in response to analyzing or assigning a disease label using any of the methods disclosed herein, an effective amount of an anti-cancer therapy or anti-cancer treatment may be administered to the subject.

In some instances, the disclosed methods for analyzing or assigning a disease label may be used for monitoring disease progression or recurrence (e.g., cancer or tumor progression or recurrence) in a subject. For example, in some instances, the methods may be used to analyze or assign a disease label in a first sample obtained from the subject at a first time point, and used to analyze or assign a disease label in a second sample obtained from the subject at a second time point, where comparison of the first analysis or assignment of the disease label and the second analysis or assignment of the disease label allows one to monitor disease progression or recurrence. In some instances, the first time point is chosen before the subject has been administered a therapy or treatment, and the second time point is chosen after the subject has been administered the therapy or treatment.

In some instances, the disclosed methods may be used for adjusting a therapy or treatment (e.g., an anti-cancer treatment or anti-cancer therapy) for a subject, e.g., by adjusting a treatment dose and/or selecting a different treatment in response to a change in the analyzing or assigning a disease label, e.g., if rejecting a predetermined disease label and/or accepting an alternate disease label.

In some instances, the analyzed or assigned disease label determined using the disclosed methods may be used as a prognostic or diagnostic indicator associated with the sample. For example, in some instances, the prognostic or diagnostic indicator may comprise an indicator of the presence of a disease (e.g., cancer) in the sample, an indicator of the probability that a disease (e.g., cancer) is present in the sample, an indicator of the probability that the subject from which the sample was derived will develop a disease (e.g., cancer) (i.e., a risk factor), or an indicator of the likelihood that the subject from which the sample was derived will respond to a particular therapy or treatment.

In some instances, the disclosed methods for analyzing or assigning a disease label may be implemented as part of a genomic profiling process that comprises identification of the presence of variant sequences at one or more gene loci in a sample derived from a subject as part of detecting, monitoring, predicting a risk factor, or selecting a treatment for a particular disease, e.g., cancer. In some instances, the variant panel selected for genomic profiling may comprise the detection of variant sequences at a selected set of gene loci. In some instances, the variant panel selected for genomic profiling may comprise detection of variant sequences at a number of gene loci through comprehensive genomic profiling (CGP), which is a next-generation sequencing (NGS) approach used to assess hundreds of genes (including relevant cancer biomarkers) in a single assay. Inclusion of the disclosed methods for analyzing or assigning a disease label as part of a genomic profiling process (or inclusion of the output from the disclosed methods for analyzing or assigning a disease label as part of the genomic profile of the subject) can improve the validity of, e.g., disease detection calls and treatment decisions, made on the basis of the genomic profile by, for example, independently confirming the disease label in a given patient sample.

In some instances, a genomic profile may comprise information on the presence of genes (or variant sequences thereof), copy number variations, epigenetic traits, proteins (or modifications thereof), and/or other biomarkers in an individual's genome and/or proteome, as well as information on the individual's corresponding phenotypic traits and the interaction between genetic or genomic traits, phenotypic traits, and environmental factors.

In some instances, a genomic profile for the subject may comprise results from a comprehensive genomic profiling (CGP) test, a nucleic acid sequencing-based test, a gene expression profiling test, a cancer hotspot panel test, a DNA methylation test, a DNA fragmentation test, an RNA fragmentation test, or any combination thereof.

In some instances, the method can further include administering or applying a treatment or therapy (e.g., an anti-cancer agent, anti-cancer treatment, or anti-cancer therapy) to the subject based on the generated genomic profile. An anti-cancer agent or anti-cancer treatment may refer to a compound that is effective in the treatment of cancer cells. Examples of anti-cancer agents or anti-cancer therapies include, but not limited to, alkylating agents, antimetabolites, natural products, hormones, chemotherapy, radiation therapy, immunotherapy, surgery, or a therapy configured to target a defect in a specific cell signaling pathway, e.g., a defect in a DNA mismatch repair (MMR) pathway.

The methods described herein can comprise selecting an anti-cancer therapy effective in treating a cancer, the method comprising: responsive to confirming a predetermined disease label for the cancer or assigning an alternate disease label for the cancer according to one or more of the methods described herein, selecting an anti-cancer therapy effective in treating the cancer. The anti-cancer therapy can be better selected based on the disease label assigned according to the methods described herein. For example, upon comparing the test sample to the database of samples, the methods may suggest that a better statistical disease label for the test sample may be prostate cancer, as opposed to an original disease label of colon cancer. An anti-cancer therapy effective in treating the cancer may, in this example, be an anti-cancer therapy effective in treating prostate cancer, rather than colon cancer.

The methods described herein can comprise: responsive to confirming a predetermined disease label or assigning an alternate disease label for the cancer according to the method of any of the methods described herein, administering to the subject an anti-cancer therapy effective in treating the cancer. The cancer can be a type of cancer. For example, in response to confirming a predetermined disease label of prostate cancer, the anti-cancer therapy effective in treating prostate cancer can be initiated or continued, as originally decided by the clinician. In contrast, for example, in response to assigning an alternate disease label for the cancer, e.g., if the alternate disease label is lung cancer, instead of an original disease label of kidney cancer, an anti-cancer therapy effective in treating lung cancer can be selected and provided to the subject, as opposed to an anti-cancer therapy effective in treating kidney cancer. In some cases, the anti-cancer therapy effective in treating the original disease label, e.g., cancer diagnosis, may be similar or identical to the anti-cancer therapy effective in treating the new disease label, e.g., cancer diagnosis.

### Samples

The disclosed methods and systems may be used with any of a variety of samples (also referred to herein as specimens) comprising nucleic acids (e.g., DNA or RNA) that are collected from a subject (e.g., a patient). Examples of a sample include, but are not limited to, a tumor sample, a tissue sample, a biopsy sample (e.g., a tissue biopsy, a liquid biopsy, or both), a blood sample (e.g., a peripheral whole blood sample), a blood plasma sample, a blood serum sample, a lymph sample, a saliva sample, a sputum sample, a urine sample, a gynecological fluid sample, a circulating tumor cell (CTC) sample, a cerebral spinal fluid (CSF) sample, a pericardial fluid sample, a pleural fluid sample, an ascites (peritoneal fluid) sample, a feces (or stool) sample, or other body fluid, secretion, and/or excretion sample (or cell sample derived therefrom). In certain instances, the sample may be frozen sample or a formalin-fixed paraffin-embedded (FFPE) sample.

In some instances, the sample may be collected by tissue resection (e.g., surgical resection), needle biopsy, bone marrow biopsy, bone marrow aspiration, skin biopsy, endoscopic biopsy, fine needle aspiration, oral swab, nasal swab, vaginal swab or a cytology smear, scrapings, washings or lavages (such as a ductal lavage or bronchoalveolar lavage), etc.

In some instances, the sample is a liquid biopsy sample, and may comprise, e.g., whole blood, blood plasma, blood serum, urine, stool, sputum, saliva, or cerebrospinal fluid. In some instances, the sample may be a liquid biopsy sample and may comprise circulating tumor cells (CTCs). In some instances, the sample may be a liquid biopsy sample and may comprise cell-free DNA (cfDNA), circulating tumor DNA (ctDNA), or any combination thereof.

In some instances, the sample may comprise one or more premalignant or malignant cells. Premalignant, as used herein, refers to a cell or tissue that is not yet malignant but is poised to become malignant. In certain instances, the sample may be acquired from a solid tumor, a soft tissue tumor, or a metastatic lesion. In certain instances, the sample may be acquired from a hematologic malignancy or pre-malignancy. In other instances, the sample may comprise a tissue or cells from a surgical margin. In certain instances, the sample may comprise tumor-infiltrating lymphocytes. In some instances, the sample may comprise one or more non-malignant cells. In some instances, the sample may be, or is part of, a primary tumor or a metastasis (e.g., a metastasis biopsy sample). In some instances, the sample may be obtained from a site (e.g., a tumor site) with the highest percentage of tumor (e.g., tumor cells) as compared to adjacent sites (e.g., sites adjacent to the tumor). In some instances, the sample may be obtained from a site (e.g., a tumor site) with the largest tumor focus (e.g., the largest number of tumor cells as visualized under a microscope) as compared to adjacent sites (e.g., sites adjacent to the tumor).

In some instances, the disclosed methods may further comprise analyzing a primary control (e.g., a normal tissue sample). In some instances, the disclosed methods may further comprise determining if a primary control is available and, if so, isolating a control nucleic acid (e.g., DNA) from said primary control. In some instances, the sample may comprise any normal control (e.g., a normal adjacent tissue (NAT)) if no primary control is available. In some instances, the sample may be or may comprise histologically normal tissue. In some instances, the method includes evaluating a sample, e.g., a histologically normal sample (e.g., from a surgical tissue margin) using the methods described herein. In some instances, the disclosed methods may further comprise acquiring a sub-sample enriched for non-tumor cells, e.g., by macro-dissecting non-tumor tissue from said NAT in a sample not accompanied by a primary control. In some instances, the disclosed methods may further comprise determining that no primary control and no NAT is available, and marking said sample for analysis without a matched control.

In some instances, samples obtained from histologically normal tissues (e.g., otherwise histologically normal surgical tissue margins) may still comprise a genetic alteration such as a variant sequence as described herein. The methods may thus further comprise re-classifying a sample based on the presence of the detected genetic alteration. In some instances, multiple samples (e.g., from different subjects) are processed simultaneously.

The disclosed methods and systems may be applied to the analysis of nucleic acids extracted from any of variety of tissue samples (or disease states thereof), e.g., solid tissue samples, soft tissue samples, metastatic lesions, or liquid biopsy samples. Examples of tissues include, but are not limited to, connective tissue, muscle tissue, nervous tissue, epithelial tissue, and blood. Tissue samples may be collected from any of the organs within an animal or human body. Examples of human organs include, but are not limited to, the brain, heart, lungs, liver, kidneys, pancreas, spleen, thyroid, mammary glands, uterus, prostate, large intestine, small intestine, bladder, bone, skin, etc.

In some instances, the nucleic acids extracted from the sample may comprise deoxyribonucleic acid (DNA) molecules. Examples of DNA that may be suitable for analysis by the disclosed methods include, but are not limited to, genomic DNA or fragments thereof, mitochondrial DNA or fragments thereof, cell-free DNA (cfDNA), and circulating tumor DNA (ctDNA). Cell-free DNA (cfDNA) is comprised of fragments of DNA that are released from normal and/or cancerous cells during apoptosis and necrosis, and circulate in the blood stream and/or accumulate in other bodily fluids. Circulating tumor DNA (ctDNA) is comprised of fragments of DNA that are released from cancerous cells and tumors that circulate in the blood stream and/or accumulate in other bodily fluids.

In some instances, DNA is extracted from nucleated cells from the sample. In some instances, a sample may have a low nucleated cellularity, e.g., when the sample is comprised mainly of erythrocytes, lesional cells that contain excessive cytoplasm, or tissue with fibrosis. In some instances, a sample with low nucleated cellularity may require more, e.g., greater, tissue volume for DNA extraction.

In some instances, the nucleic acids extracted from the sample may comprise ribonucleic acid (RNA) molecules. Examples of RNA that may be suitable for analysis by the disclosed methods include, but are not limited to, total cellular RNA, total cellular RNA after depletion of certain abundant RNA sequences (e.g., ribosomal RNAs), cell-free RNA (cfRNA), messenger RNA (mRNA) or fragments thereof, the poly(A)-tailed mRNA fraction of the total RNA, ribosomal RNA (rRNA) or fragments thereof, transfer RNA (tRNA) or fragments thereof, and mitochondrial RNA or fragments thereof. In some instances, RNA may be extracted from the sample and converted to complementary DNA (cDNA) using, e.g., a reverse transcription reaction. In some instances, the cDNA is produced by random-primed cDNA synthesis methods. In other instances, the cDNA synthesis is initiated at the poly(A) tail of mature mRNAs by priming with oligo(dT)-containing oligonucleotides. Methods for depletion, poly(A) enrichment, and cDNA synthesis are well known to those of skill in the art.

In some instances, the sample may comprise a tumor content (e.g., comprising tumor cells or tumor cell nuclei), or a non-tumor content (e.g., immune cells, fibroblasts, and other non-tumor cells). In some instances, the tumor content of the sample may constitute a sample metric. In some instances, the sample may comprise a tumor content of at least 5-50%, 10-40%, 15-25%, or 20-30% tumor cell nuclei. In some instances, the sample may comprise a tumor content of at least 5%, at least 10%, at least 20%, at least 30%, at least 40%, or at least 50% tumor cell nuclei. In some instances, the percent tumor cell nuclei (e.g., sample fraction) is determined (e.g., calculated) by dividing the number of tumor cells in the sample by the total number of all cells within the sample that have nuclei. In some instances, for example when the sample is a liver sample comprising hepatocytes, a different tumor content calculation may be required due to the presence of hepatocytes having nuclei with twice, or more than twice, the DNA content of other, e.g., non-hepatocyte, somatic cell nuclei. In some instances, the sensitivity of detection of a genetic alteration, e.g., a variant sequence, or a determination of, e.g., microsatellite instability, may depend on the tumor content of the sample. For example, a sample having a lower tumor content can result in lower sensitivity of detection for a given size sample.

In some instances, as noted above, the sample comprises nucleic acid (e.g., DNA, RNA (or a cDNA derived from the RNA), or both), e.g., from a tumor or from normal tissue. In certain instances, the sample may further comprise a non-nucleic acid component, e.g., cells, protein, carbohydrate, or lipid, e.g., from the tumor or normal tissue.

### Subjects

In some instances, the sample is obtained (e.g., collected) from a subject (e.g., patient) with a condition or disease (e.g., a hyperproliferative disease or a non-cancer indication) or suspected of having the condition or disease. In some instances, the hyperproliferative disease is a cancer. In some instances, the cancer is a solid tumor or a metastatic form thereof. In some instances, the cancer is a hematological cancer, e.g., a leukemia or lymphoma.

In some instances, the subject has a cancer or is at risk of having a cancer. For example, in some instances, the subject has a genetic predisposition to a cancer (e.g., having a genetic mutation that increases his or her baseline risk for developing a cancer). In some instances, the subject has been exposed to an environmental perturbation (e.g., radiation or a chemical) that increases his or her risk for developing a cancer. In some instances, the subject is in need of being monitored for development of a cancer. In some instances, the subject is in need of being monitored for cancer progression or regression, e.g., after being treated with an anti-cancer therapy (or anti-cancer treatment). In some instances, the subject is in need of being monitored for relapse of cancer. In some instances, the subject is in need of being monitored for minimum residual disease (MRD). In some instances, the subject has been, or is being treated, for cancer. In some instances, the subject has not been treated with an anti-cancer therapy (or anti-cancer treatment).

In some instances, the subject (e.g., a patient) is being treated, or has been previously treated, with one or more targeted therapies. In some instances, e.g., for a patient who has been previously treated with a targeted therapy, a post-targeted therapy sample (e.g., specimen) is obtained (e.g., collected). In some instances, the post-targeted therapy sample is a sample obtained after the completion of the targeted therapy.

In some instances, the patient has not been previously treated with a targeted therapy. In some instances, e.g., for a patient who has not been previously treated with a targeted therapy, the sample comprises a resection, e.g., an original resection, or a resection following recurrence (e.g., following a disease recurrence post-therapy).

### Cancers

In some instances, the sample is acquired from a subject having a cancer. Exemplary cancers include, but are not limited to, B cell cancer (e.g., multiple myeloma), melanomas, breast cancer, lung cancer (such as non-small cell lung carcinoma or NSCLC), bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, adenocarcinomas, inflammatory myofibroblastic tumors, gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancers, essential thrombocythemia, agnogenic myeloid metaplasia, hypereosinophilic syndrome, systemic mastocytosis, familiar hypereosinophilia, chronic eosinophilic leukemia, neuroendocrine cancers, carcinoid tumors, and the like.

In some instances, the cancer comprises acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR and MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), a gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma, Hodgkin lymphoma, juvenile idiopathic arthritis, lupus erythematosus, a mantle cell lymphoma, medullary thyroid cancer, melanoma, a melanoma with a BRAF V600 mutation, a melanoma with a BRAF V600E or V600K mutation, Merkel cell carcinoma, multicentric Castleman's disease, multiple hematologic malignancies including Philadelphia chromosome-positive ALL and CML, multiple myeloma, myelofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROS1 gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, rheumatoid arthritis, a small lymphocytic lymphoma, a soft tissue sarcoma, a solid tumor (MSI-H/dMMR), a squamous cell cancer of the head and neck, a squamous non-small cell lung cancer, thyroid cancer, a thyroid carcinoma, urothelial cancer, a urothelial carcinoma, or Waldenstrom's macroglobulinemia.

In some instances, the cancer is a hematologic malignancy (or premaligancy). As used herein, a hematologic malignancy refers to a tumor of the hematopoietic or lymphoid tissues, e.g., a tumor that affects blood, bone marrow, or lymph nodes. Exemplary hematologic malignancies include, but are not limited to, leukemia (e.g., acute lymphoblastic leukemia (ALL), acute myeloid leukemia (AML), chronic lymphocytic leukemia (CLL), chronic myelogenous leukemia (CML), hairy cell leukemia, acute monocytic leukemia (AMoL), chronic myelomonocytic leukemia (CMML), juvenile myelomonocytic leukemia (JMML), or large granular lymphocytic leukemia), lymphoma (e.g., AIDS-related lymphoma, cutaneous T-cell lymphoma, Hodgkin lymphoma (e.g., classical Hodgkin lymphoma or nodular lymphocyte-predominant Hodgkin lymphoma), mycosis fungoides, non-Hodgkin lymphoma (e.g., B-cell non-Hodgkin lymphoma (e.g., Burkitt lymphoma, small lymphocytic lymphoma (CLL/SLL), diffuse large B-cell lymphoma, follicular lymphoma, immunoblastic large cell lymphoma, precursor B-lymphoblastic lymphoma, or mantle cell lymphoma) or T-cell non-Hodgkin lymphoma (mycosis fungoides, anaplastic large cell lymphoma, or precursor T-lymphoblastic lymphoma)), primary central nervous system lymphoma, Sézary syndrome, Waldenström macroglobulinemia), chronic myeloproliferative neoplasm, Langerhans cell histiocytosis, multiple myeloma/plasma cell neoplasm, myelodysplastic syndrome, or myelodysplastic/myeloproliferative neoplasm.

### Nucleic Acid Extraction and Processing

DNA or RNA may be extracted from tissue samples, biopsy samples, blood samples, or other bodily fluid samples using any of a variety of techniques known to those of skill in the art (see, e.g., Example 1 of International Patent Application Publication No. WO 2012/092426; Tan, et al. (2009), “DNA, RNA, and Protein Extraction: The Past and The Present”, J. Biomed. Biotech. 2009:574398; the technical literature for the Maxwell® 16 LEV Blood DNA Kit (Promega Corporation, Madison, WI); and the Maxwell 16 Buccal Swab LEV DNA Purification Kit Technical Manual (Promega Literature #TM333, Jan. 1, 2011, Promega Corporation, Madison, WI)). Protocols for RNA isolation are disclosed in, e.g., the Maxwell® 16 Total RNA Purification Kit Technical Bulletin (Promega Literature #TB351, August 2009, Promega Corporation, Madison, WI).

A typical DNA extraction procedure, for example, comprises (i) collection of the fluid sample, cell sample, or tissue sample from which DNA is to be extracted, (ii) disruption of cell membranes (i.e., cell lysis), if necessary, to release DNA and other cytoplasmic components, (iii) treatment of the fluid sample or lysed sample with a concentrated salt solution to precipitate proteins, lipids, and RNA, followed by centrifugation to separate out the precipitated proteins, lipids, and RNA, and (iv) purification of DNA from the supernatant to remove detergents, proteins, salts, or other reagents used during the cell membrane lysis step.

Disruption of cell membranes may be performed using a variety of mechanical shear (e.g., by passing through a French press or fine needle) or ultrasonic disruption techniques. The cell lysis step often comprises the use of detergents and surfactants to solubilize lipids the cellular and nuclear membranes. In some instances, the lysis step may further comprise use of proteases to break down protein, and/or the use of an RNase for digestion of RNA in the sample.

Examples of suitable techniques for DNA purification include, but are not limited to, (i) precipitation in ice-cold ethanol or isopropanol, followed by centrifugation (precipitation of DNA may be enhanced by increasing ionic strength, e.g., by addition of sodium acetate), (ii) phenol-chloroform extraction, followed by centrifugation to separate the aqueous phase containing the nucleic acid from the organic phase containing denatured protein, and (iii) solid phase chromatography where the nucleic acids adsorb to the solid phase (e.g., silica or other) depending on the pH and salt concentration of the buffer.

In some instances, cellular and histone proteins bound to the DNA may be removed either by adding a protease or by having precipitated the proteins with sodium or ammonium acetate, or through extraction with a phenol-chloroform mixture prior to a DNA precipitation step.

In some instances, DNA may be extracted using any of a variety of suitable commercial DNA extraction and purification kits. Examples include, but are not limited to, the QIAamp (for isolation of genomic DNA from human samples) and DNAeasy (for isolation of genomic DNA from animal or plant samples) kits from Qiagen (Germantown, MD) or the Maxwell® and ReliaPrep™ series of kits from Promega (Madison, WI).

As noted above, in some instances the sample may comprise a formalin-fixed (also known as formaldehyde-fixed, or paraformaldehyde-fixed), paraffin-embedded (FFPE) tissue preparation. For example, the FFPE sample may be a tissue sample embedded in a matrix, e.g., an FFPE block. Methods to isolate nucleic acids (e.g., DNA) from formaldehyde- or paraformaldehyde-fixed, paraffin-embedded (FFPE) tissues are disclosed in, e.g., Cronin, et al., (2004) Am J Pathol. 164(1):35-42; Masuda, et al., (1999) Nucleic Acids Res. 27(22):4436-4443; Specht, et al., (2001) Am J Pathol. 158(2):419-429; the Ambion RecoverAll™ Total Nucleic Acid Isolation Protocol (Ambion, Cat. No. AM1975, September 2008); the Maxwell® 16 FFPE Plus LEV DNA Purification Kit Technical Manual (Promega Literature #TM349, February 2011); the E.Z.N.A.® FFPE DNA Kit Handbook (OMEGA bio-tek, Norcross, GA, product numbers D3399-00, D3399-01, and D3399-02, June 2009); and the QIAamp® DNA FFPE Tissue Handbook (Qiagen, Cat. No. 37625, October 2007). For example, the RecoverAll™ Total Nucleic Acid Isolation Kit uses xylene at elevated temperatures to solubilize paraffin-embedded samples and a glass-fiber filter to capture nucleic acids. The Maxwell@16 FFPE Plus LEV DNA Purification Kit is used with the Maxwell@16 Instrument for purification of genomic DNA from 1 to 10 μm sections of FFPE tissue. DNA is purified using silica-clad paramagnetic particles (PMPs), and eluted in low elution volume. The E.Z.N.A.® FFPE DNA Kit uses a spin column and buffer system for isolation of genomic DNA. QIAamp® DNA FFPE Tissue Kit uses QIAamp® DNA Micro technology for purification of genomic and mitochondrial DNA.

In some instances, the disclosed methods may further comprise determining or acquiring a yield value for the nucleic acid extracted from the sample and comparing the determined value to a reference value. For example, if the determined or acquired value is less than the reference value, the nucleic acids may be amplified prior to proceeding with library construction. In some instances, the disclosed methods may further comprise determining or acquiring a value for the size (or average size) of nucleic acid fragments in the sample, and comparing the determined or acquired value to a reference value, e.g., a size (or average size) of at least 100, 200, 300, 400, 500, 600, 700, 800, 900, or 1000 base pairs (bps). In some instances, one or more parameters described herein may be adjusted or selected in response to this determination.

After isolation, the nucleic acids are typically dissolved in a slightly alkaline buffer, e.g., Tris-EDTA (TE) buffer, or in ultra-pure water. In some instances, the isolated nucleic acids (e.g., genomic DNA) may be fragmented or sheared by using any of a variety of techniques known to those of skill in the art. For example, genomic DNA can be fragmented by physical shearing methods, enzymatic cleavage methods, chemical cleavage methods, and other methods known to those of skill in the art. Methods for DNA shearing are described in Example 4 in International Patent Application Publication No. WO 2012/092426. In some instances, alternatives to DNA shearing methods can be used to avoid a ligation step during library preparation.

### Library Preparation

In some instances, the nucleic acids isolated from the sample may be used to construct a library (e.g., a nucleic acid library as described herein). In some instances, the nucleic acids are fragmented using any of the methods described above, optionally subjected to repair of chain end damage, and optionally ligated to synthetic adapters, primers, and/or barcodes (e.g., amplification primers, sequencing adapters, flow cell adapters, substrate adapters, sample barcodes or indexes, and/or unique molecular identifier sequences), size-selected (e.g., by preparative gel electrophoresis), and/or amplified (e.g., using PCR, a non-PCR amplification technique, or an isothermal amplification technique). In some instances, the fragmented and adapter-ligated group of nucleic acids is used without explicit size selection or amplification prior to hybridization-based selection of target sequences. In some instances, the nucleic acid is amplified by any of a variety of specific or non-specific nucleic acid amplification methods known to those of skill in the art. In some instances, the nucleic acids are amplified, e.g., by a whole-genome amplification method such as random-primed strand-displacement amplification. Examples of nucleic acid library preparation techniques for next-generation sequencing are described in, e.g., van Dijk, et al. (2014), Exp. Cell Research 322:12-20, and Illumina's genomic DNA sample preparation kit.

In some instances, the resulting nucleic acid library may contain all or substantially all of the complexity of the genome. The term “substantially all” in this context refers to the possibility that there can in practice be some unwanted loss of genome complexity during the initial steps of the procedure. The methods described herein also are useful in cases where the nucleic acid library comprises a portion of the genome, e.g., where the complexity of the genome is reduced by design. In some instances, any selected portion of the genome can be used with a method described herein. For example, in certain embodiments, the entire exome or a subset thereof is isolated. In some instances, the library may include at least 95%, 90%, 80%, 70%, 60%, 50%, 40%, 30%, 20%, 10%, or 5% of the genomic DNA. In some instances, the library may consist of cDNA copies of genomic DNA that includes copies of at least 95%, 90%, 80%, 70%, 60%, 50%, 40%, 30%, 20%, 10%, or 5% of the genomic DNA. In certain instances, the amount of nucleic acid used to generate the nucleic acid library may be less than 5 micrograms, less than 1 microgram, less than 500 ng, less than 200 ng, less than 100 ng, less than 50 ng, less than 10 ng, less than 5 ng, or less than 1 ng.

In some instances, a library (e.g., a nucleic acid library) includes a collection of nucleic acid molecules. As described herein, the nucleic acid molecules of the library can include a target nucleic acid molecule (e.g., a tumor nucleic acid molecule, a reference nucleic acid molecule and/or a control nucleic acid molecule; also referred to herein as a first, second and/or third nucleic acid molecule, respectively). The nucleic acid molecules of the library can be from a single subject or individual. In some instances, a library can comprise nucleic acid molecules derived from more than one subject (e.g., 2, 3, 4, 5, 6, 7, 8, 9, 10, 20, 30 or more subjects). For example, two or more libraries from different subjects can be combined to form a library having nucleic acid molecules from more than one subject (where the nucleic acid molecules derived from each subject are optionally ligated to a unique sample barcode corresponding to a specific subject). In some instances, the subject is a human having, or at risk of having, a cancer or tumor.

In some instances, the library (or a portion thereof) may comprise one or more subgenomic intervals. In some instances, a subgenomic interval can be a single nucleotide position, e.g., a nucleotide position for which a variant at the position is associated (positively or negatively) with a tumor phenotype. In some instances, a subgenomic interval comprises more than one nucleotide position. Such instances include sequences of at least 2, 5, 10, 50, 100, 150, 250, or more than 250 nucleotide positions in length. Subgenomic intervals can comprise, e.g., one or more entire genes (or portions thereof), one or more exons or coding sequences (or portions thereof), one or more introns (or portion thereof), one or more microsatellite region (or portions thereof), or any combination thereof. A subgenomic interval can comprise all or a part of a fragment of a naturally occurring nucleic acid molecule, e.g., a genomic DNA molecule. For example, a subgenomic interval can correspond to a fragment of genomic DNA which is subjected to a sequencing reaction. In some instances, a subgenomic interval is a continuous sequence from a genomic source. In some instances, a subgenomic interval includes sequences that are not contiguous in the genome, e.g., subgenomic intervals in cDNA can include exon-exon junctions formed as a result of splicing. In some instances, the subgenomic interval comprises a tumor nucleic acid molecule. In some instances, the subgenomic interval comprises a non-tumor nucleic acid molecule.

### Targeting Gene Loci for Analysis

The methods described herein can be used in combination with, or as part of, a method for evaluating a plurality or set of subject intervals (e.g., target sequences), e.g., from a set of genomic loci (e.g., gene loci or fragments thereof), as described herein.

In some instances, the set of genomic loci evaluated by the disclosed methods comprises a plurality of, e.g., genes, which in mutant form, are associated with an effect on cell division, growth or survival, or are associated with a cancer, e.g., a cancer described herein.

In some instances, the set of gene loci evaluated by the disclosed methods comprises at least 1, at least 2, at least 3, at least 4, at least 5, at least 6, at least 7, at least 8, at least 9, at least 10, at least 20, at least 30, at least 40, at least 50, at least 60, at least 70, at least 80, at least 90, at least 100, or more than 100 gene loci.

In some instances, the selected gene loci (also referred to herein as target gene loci or target sequences), or fragments thereof, may include subject intervals comprising non-coding sequences, coding sequences, intragenic regions, or intergenic regions of the subject genome. For example, the subject intervals can include a non-coding sequence or fragment thereof (e.g., a promoter sequence, enhancer sequence, 5′ untranslated region (5′ UTR), 3′ untranslated region (3′ UTR), or a fragment thereof), a coding sequence of fragment thereof, an exon sequence or fragment thereof, an intron sequence or a fragment thereof.

### Target Capture Reagents

The methods described herein may comprise contacting a nucleic acid library with a plurality of target capture reagents in order to select and capture a plurality of specific target sequences (e.g., gene sequences or fragments thereof) for analysis. In some instances, a target capture reagent (i.e., a molecule which can bind to and thereby allow capture of a target molecule) is used to select the subject intervals to be analyzed. For example, a target capture reagent can be a bait molecule, e.g., a nucleic acid molecule (e.g., a DNA molecule or RNA molecule) which can hybridize to (i.e., is complementary to) a target molecule, and thereby allows capture of the target nucleic acid. In some instances, the target capture reagent, e.g., a bait molecule (or bait sequence), is a capture oligonucleotide (or capture probe). In some instances, the target nucleic acid is a genomic DNA molecule, an RNA molecule, a cDNA molecule derived from an RNA molecule, a microsatellite DNA sequence, and the like. In some instances, the target capture reagent is suitable for solution-phase hybridization to the target. In some instances, the target capture reagent is suitable for solid-phase hybridization to the target. In some instances, the target capture reagent is suitable for both solution-phase and solid-phase hybridization to the target. The design and construction of target capture reagents is described in more detail in, e.g., International Patent Application Publication No. WO 2020/236941, the entire content of which is incorporated herein by reference.

The methods described herein provide for optimized sequencing of a large number of genomic loci (e.g., genes or gene products (e.g., mRNA), microsatellite loci, etc.) from samples (e.g., cancerous tissue specimens, liquid biopsy samples, and the like) from one or more subjects by the appropriate selection of target capture reagents to select the target nucleic acid molecules to be sequenced. In some instances, a target capture reagent may hybridize to a specific target locus, e.g., a specific target gene locus or fragment thereof. In some instances, a target capture reagent may hybridize to a specific group of target loci, e.g., a specific group of gene loci or fragments thereof. In some instances, a plurality of target capture reagents comprising a mix of target-specific and/or group-specific target capture reagents may be used.

In some instances, the number of target capture reagents (e.g., bait molecules) in the plurality of target capture reagents (e.g., a bait set) contacted with a nucleic acid library to capture a plurality of target sequences for nucleic acid sequencing is greater than 10, greater than 50, greater than 100, greater than 200, greater than 300, greater than 400, greater than 500, greater than 600, greater than 700, greater than 800, greater than 900, greater than 1,000, greater than 1,250, greater than 1,500, greater than 1,750, greater than 2,000, greater than 3,000, greater than 4,000, greater than 5,000, greater than 10,000, greater than 25,000, or greater than 50,000.

In some instances, the overall length of the target capture reagent sequence can be between about 70 nucleotides and 1000 nucleotides. In one instance, the target capture reagent length is between about 100 and 300 nucleotides, 110 and 200 nucleotides, or 120 and 170 nucleotides, in length. In addition to those mentioned above, intermediate oligonucleotide lengths of about 70, 80, 90, 100, 110, 120, 130, 140, 150, 160, 170, 180, 190, 200, 210, 220, 230, 240, 250, 300, 400, 500, 600, 700, 800, and 900 nucleotides in length can be used in the methods described herein. In some embodiments, oligonucleotides of about 70, 80, 90, 100, 110, 120, 130, 140, 150, 160, 170, 180, 190, 200, 210, 220, or 230 bases can be used.

In some instances, each target capture reagent sequence can include: (i) a target-specific capture sequence (e.g., a gene locus or microsatellite locus-specific complementary sequence), (ii) an adapter, primer, barcode, and/or unique molecular identifier sequence, and (iii) universal tails on one or both ends. As used herein, the term “target capture reagent” can refer to the target-specific target capture sequence or to the entire target capture reagent oligonucleotide including the target-specific target capture sequence.

In some instances, the target-specific capture sequences in the target capture reagents are between about 40 nucleotides and 1000 nucleotides in length. In some instances, the target-specific capture sequence is between about 70 nucleotides and 300 nucleotides in length. In some instances, the target-specific sequence is between about 100 nucleotides and 200 nucleotides in length. In yet other instances, the target-specific sequence is between about 120 nucleotides and 170 nucleotides in length, typically 120 nucleotides in length. Intermediate lengths in addition to those mentioned above also can be used in the methods described herein, such as target-specific sequences of about 40, 50, 60, 70, 80, 90, 100, 110, 120, 130, 140, 150, 160, 170, 180, 190, 200, 210, 220, 230, 240, 250, 300, 400, 500, 600, 700, 800, and 900 nucleotides in length, as well as target-specific sequences of lengths between the above-mentioned lengths.

In some instances, the target capture reagent may be designed to select a subject interval containing one or more rearrangements, e.g., an intron containing a genomic rearrangement. In such instances, the target capture reagent is designed such that repetitive sequences are masked to increase the selection efficiency. In those instances where the rearrangement has a known juncture sequence, complementary target capture reagents can be designed to recognize the juncture sequence to increase the selection efficiency.

In some instances, the disclosed methods may comprise the use of target capture reagents designed to capture two or more different target categories, each category having a different target capture reagent design strategy. In some instances, the hybridization-based capture methods and target capture reagent compositions disclosed herein may provide for the capture and homogeneous coverage of a set of target sequences, while minimizing coverage of genomic sequences outside of the targeted set of sequences. In some instances, the target sequences may include the entire exome of genomic DNA or a selected subset thereof. In some instances, the target sequences may include, e.g., a large chromosomal region (e.g., a whole chromosome arm). The methods and compositions disclosed herein provide different target capture reagents for achieving different sequencing depths and patterns of coverage for complex sets of target nucleic acid sequences.

Typically, DNA molecules are used as target capture reagent sequences, although RNA molecules can also be used. In some instances, a DNA molecule target capture reagent can be single stranded DNA (ssDNA) or double-stranded DNA (dsDNA). In some instances, an RNA-DNA duplex is more stable than a DNA-DNA duplex and therefore provides for potentially better capture of nucleic acids.

In some instances, the disclosed methods comprise providing a selected set of nucleic acid molecules (e.g., a library catch) captured from one or more nucleic acid libraries. For example, the method may comprise: providing one or a plurality of nucleic acid libraries, each comprising a plurality of nucleic acid molecules (e.g., a plurality of target nucleic acid molecules and/or reference nucleic acid molecules) extracted from one or more samples from one or more subjects; contacting the one or a plurality of libraries (e.g., in a solution-based hybridization reaction) with one, two, three, four, five, or more than five pluralities of target capture reagents (e.g., oligonucleotide target capture reagents) to form a hybridization mixture comprising a plurality of target capture reagent/nucleic acid molecule hybrids; separating the plurality of target capture reagent/nucleic acid molecule hybrids from said hybridization mixture, e.g., by contacting said hybridization mixture with a binding entity that allows for separation of said plurality of target capture reagent/nucleic acid molecule hybrids from the hybridization mixture, thereby providing a library catch (e.g., a selected or enriched subgroup of nucleic acid molecules from the one or a plurality of libraries).

In some instances, the disclosed methods may further comprise amplifying the library catch (e.g., by performing PCR). In other instances, the library catch is not amplified.

In some instances, the target capture reagents can be part of a kit which can optionally comprise instructions, standards, buffers or enzymes or other reagents.

### Hybridization Conditions

As noted above, the methods disclosed herein may include the step of contacting the library (e.g., the nucleic acid library) with a plurality of target capture reagents to provide a selected library target nucleic acid sequences (i.e., the library catch). The contacting step can be effected in, e.g., solution-based hybridization. In some instances, the method includes repeating the hybridization step for one or more additional rounds of solution-based hybridization. In some instances, the method further includes subjecting the library catch to one or more additional rounds of solution-based hybridization with the same or a different collection of target capture reagents.

In some instances, the contacting step is effected using a solid support, e.g., an array. Suitable solid supports for hybridization are described in, e.g., Albert, T. J. et al. (2007) Nat. Methods 4(11):903-5; Hodges, E. et al. (2007) Nat. Genet. 39(12):1522-7; and Okou, D. T. et al. (2007) Nat. Methods 4(11):907-9, the contents of which are incorporated herein by reference in their entireties.

Hybridization methods that can be adapted for use in the methods herein are described in the art, e.g., as described in International Patent Application Publication No. WO 2012/092426. Methods for hybridizing target capture reagents to a plurality of target nucleic acids are described in more detail in, e.g., International Patent Application Publication No. WO 2020/236941, the entire content of which is incorporated herein by reference.

### Sequencing Methods

The methods and systems disclosed herein can be used in combination with, or as part of, a method or system for sequencing nucleic acids (e.g., a next-generation sequencing system) to generate a plurality of sequence reads that overlap one or more gene loci within a subgenomic interval in the sample and thereby determine, e.g., gene allele sequences at a plurality of gene loci. “Next-generation sequencing” (or “NGS”) as used herein may also be referred to as “massively parallel sequencing” (or “MPS”), and refers to any sequencing method that determines the nucleotide sequence of either individual nucleic acid molecules (e.g., as in single molecule sequencing) or clonally expanded proxies for individual nucleic acid molecules in a high throughput fashion (e.g., wherein greater than 10³, 10⁴, 10⁵ or more than 10⁵ molecules are sequenced simultaneously).

Next-generation sequencing methods are known in the art, and are described in, e.g., Metzker, M. (2010) Nature Biotechnology Reviews 11:31-46, which is incorporated herein by reference. Other examples of sequencing methods suitable for use when implementing the methods and systems disclosed herein are described in, e.g., International Patent Application Publication No. WO 2012/092426. In some instances, the sequencing may comprise, for example, whole genome sequencing (WGS), whole exome sequencing, targeted sequencing, or direct sequencing. In some instances, sequencing may be performed using, e.g., Sanger sequencing. In some instances, the sequencing may comprise a paired-end sequencing technique that allows both ends of a fragment to be sequenced and generates high-quality, alignable sequence data for detection of, e.g., genomic rearrangements, repetitive sequence elements, gene fusions, and novel transcripts.

The disclosed methods and systems may be implemented using sequencing platforms such as the Roche 454, Illumina Solexa, ABI-SOLiD, ION Torrent, Complete Genomics, Pacific Bioscience, Helicos, and/or the Polonator platform. In some instances, sequencing may comprise Illumina MiSeq sequencing. In some instances, sequencing may comprise Illumina HiSeq sequencing. In some instances, sequencing may comprise Illumina NovaSeq sequencing. Optimized methods for sequencing a large number of target genomic loci in nucleic acids extracted from a sample are described in more detail in, e.g., International Patent Application Publication No. WO 2020/236941, the entire content of which is incorporated herein by reference.

In certain instances, the disclosed methods comprise one or more of the steps of: (a) acquiring a library comprising a plurality of normal and/or tumor nucleic acid molecules from a sample; (b) simultaneously or sequentially contacting the library with one, two, three, four, five, or more than five pluralities of target capture reagents under conditions that allow hybridization of the target capture reagents to the target nucleic acid molecules, thereby providing a selected set of captured normal and/or tumor nucleic acid molecules (i.e., a library catch); (c) separating the selected subset of the nucleic acid molecules (e.g., the library catch) from the hybridization mixture, e.g., by contacting the hybridization mixture with a binding entity that allows for separation of the target capture reagent/nucleic acid molecule hybrids from the hybridization mixture, (d) sequencing the library catch to acquiring a plurality of reads (e.g., sequence reads) that overlap one or more subject intervals (e.g., one or more target sequences) from said library catch that may comprise a mutation (or alteration), e.g., a variant sequence comprising a somatic mutation or germline mutation; (e) aligning said sequence reads using an alignment method as described elsewhere herein; and/or (f) assigning a nucleotide value for a nucleotide position in the subject interval (e.g., calling a mutation using, e.g., a Bayesian method or other method described herein) from one or more sequence reads of the plurality.

In some instances, acquiring sequence reads for one or more subject intervals may comprise sequencing at least 1, at least 5, at least 10, at least 20, at least 30, at least 40, at least 50, at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, at least 1,000, at least 1,250, at least 1,500, at least 1,750, at least 2,000, at least 2,250, at least 2,500, at least 2,750, at least 3,000, at least 3,500, at least 4,000, at least 4,500, or at least 5,000 loci, e.g., genomic loci, gene loci, microsatellite loci, etc. In some instances, acquiring a sequence read for one or more subject intervals may comprise sequencing a subject interval for any number of loci within the range described in this paragraph, e.g., for at least 2,850 gene loci.

In some instances, acquiring a sequence read for one or more subject intervals comprises sequencing a subject interval with a sequencing method that provides a sequence read length (or average sequence read length) of at least 20 bases, at least 30 bases, at least 40 bases, at least 50 bases, at least 60 bases, at least 70 bases, at least 80 bases, at least 90 bases, at least 100 bases, at least 120 bases, at least 140 bases, at least 160 bases, at least 180 bases, at least 200 bases, at least 220 bases, at least 240 bases, at least 260 bases, at least 280 bases, at least 300 bases, at least 320 bases, at least 340 bases, at least 360 bases, at least 380 bases, or at least 400 bases. In some instances, acquiring a sequence read for the one or more subject intervals may comprise sequencing a subject interval with a sequencing method that provides a sequence read length (or average sequence read length) of any number of bases within the range described in this paragraph, e.g., a sequence read length (or average sequence read length) of 56 bases.

In some instances, acquiring a sequence read for one or more subject intervals may comprise sequencing with at least 100× or more coverage (or depth) on average. In some instances, acquiring a sequence read for one or more subject intervals may comprise sequencing with at least 100×, at least 150×, at least 200×, at least 250×, at least 500×, at least 750×, at least 1,000×, at least 1,500×, at least 2,000×, at least 2,500×, at least 3,000×, at least 3,500×, at least 4,000×, at least 4,500×, at least 5,000×, at least 5,500×, or at least 6,000× or more coverage (or depth) on average. In some instances, acquiring a sequence read for one or more subject intervals may comprise sequencing with an average coverage (or depth) having any value within the range of values described in this paragraph, e.g., at least 160×.

In some instances, acquiring a read for the one or more subject intervals comprises sequencing with an average sequencing depth having any value ranging from at least 100× to at least 6,000× for greater than about 90%, 92%, 94%, 95%, 96%, 97%, 98%, or 99% of the gene loci sequenced. For example, in some instances acquiring a read for the subject interval comprises sequencing with an average sequencing depth of at least 125× for at least 99% of the gene loci sequenced. As another example, in some instances acquiring a read for the subject interval comprises sequencing with an average sequencing depth of at least 4,100× for at least 95% of the gene loci sequenced.

In some instances, the relative abundance of a nucleic acid species in the library can be estimated by counting the relative number of occurrences of their cognate sequences (e.g., the number of sequence reads for a given cognate sequence) in the data generated by the sequencing experiment.

In some instances, the disclosed methods and systems provide nucleotide sequences for a set of subject intervals (e.g., gene loci), as described herein. In certain instances, the sequences are provided without using a method that includes a matched normal control (e.g., a wild-type control) and/or a matched tumor control (e.g., primary versus metastatic).

In some instances, the level of sequencing depth as used herein (e.g., an X-fold level of sequencing depth) refers to the number of reads (e.g., unique reads) obtained after detection and removal of duplicate reads (e.g., PCR duplicate reads). In other instances, duplicate reads are evaluated, e.g., to support detection of copy number alteration (CNAs).

### Alignment

Alignment is the process of matching a read with a location, e.g., a genomic location or locus. In some instances, NGS reads may be aligned to a known reference sequence (e.g., a wild-type sequence). In some instances, NGS reads may be assembled de novo. Methods of sequence alignment for NGS reads are described in, e.g., Trapnell, C. and Salzberg, S. L. Nature Biotech., 2009, 27:455-457. Examples of de novo sequence assemblies are described in, e.g., Warren R., et al., Bioinformatics, 2007, 23:500-501; Butler, J. et al., Genome Res., 2008, 18:810-820; and Zerbino, D. R. and Birney, E., Genome Res., 2008, 18:821-829. Optimization of sequence alignment is described in the art, e.g., as set out in International Patent Application Publication No. WO 2012/092426. Additional description of sequence alignment methods is provided in, e.g., International Patent Application Publication No. WO 2020/236941, the entire content of which is incorporated herein by reference.

Misalignment (e.g., the placement of base-pairs from a short read at incorrect locations in the genome), e.g., misalignment of reads due to sequence context (e.g., the presence of repetitive sequence) around an actual cancer mutation can lead to reduction in sensitivity of mutation detection, can lead to a reduction in sensitivity of mutation detection, as reads for the alternate allele may be shifted off the histogram peak of alternate allele reads. Other examples of sequence context that may cause misalignment include short-tandem repeats, interspersed repeats, low complexity regions, insertions—deletions (indels), and paralogs. If the problematic sequence context occurs where no actual mutation is present, misalignment may introduce artifactual reads of “mutated” alleles by placing reads of actual reference genome base sequences at the wrong location. Because mutation-calling algorithms for multigene analysis should be sensitive to even low-abundance mutations, sequence misalignments may increase false positive discovery rates and/or reduce specificity.

In some instances, the methods and systems disclosed herein may integrate the use of multiple, individually-tuned, alignment methods or algorithms to optimize base-calling performance in sequencing methods, particularly in methods that rely on massively parallel sequencing (MPS) of a large number of diverse genetic events at a large number of diverse genomic loci. In some instances, the disclosed methods and systems may comprise the use of one or more global alignment algorithms. In some instances, the disclosed methods and systems may comprise the use of one or more local alignment algorithms. Examples of alignment algorithms that may be used include, but are not limited to, the Burrows-Wheeler Alignment (BWA) software bundle (see, e.g., Li, et al. (2009), “Fast and Accurate Short Read Alignment with Burrows-Wheeler Transform”, Bioinformatics 25:1754-60; Li, et al. (2010), Fast and Accurate Long-Read Alignment with Burrows-Wheeler Transform”, Bioinformatics epub. PMID: 20080505), the Smith-Waterman algorithm (see, e.g., Smith, et al. (1981), “Identification of Common Molecular Subsequences”, J. Molecular Biology 147(1):195-197), the Striped Smith-Waterman algorithm (see, e.g., Farrar (2007), “Striped Smith-Waterman Speeds Database Searches Six Times Over Other SIMD Implementations”, Bioinformatics 23(2):156-161), the Needleman-Wunsch algorithm (Needleman, et al. (1970) “A General Method Applicable to the Search for Similarities in the Amino Acid Sequence of Two Proteins”, J. Molecular Biology 48(3):443-53), or any combination thereof.

In some instances, the methods and systems disclosed herein may also comprise the use of a sequence assembly algorithm, e.g., the Arachne sequence assembly algorithm (see, e.g., Batzoglou, et al. (2002), “ARACHNE: A Whole-Genome Shotgun Assembler”, Genome Res. 12:177-189).

In some instances, the alignment method used to analyze sequence reads is not individually customized or tuned for detection of different variants (e.g., point mutations, insertions, deletions, and the like) at different genomic loci. In some instances, different alignment methods are used to analyze reads that are individually customized or tuned for detection of at least a subset of the different variants detected at different genomic loci. In some instances, different alignment methods are used to analyze reads that are individually customized or tuned to detect each different variant at different genomic loci. In some instances, tuning can be a function of one or more of: (i) the genetic locus (e.g., gene loci, microsatellite locus, or other subject interval) being sequenced, (ii) the tumor type associated with the sample, (iii) the variant being sequenced, or (iv) a characteristic of the sample or the subject. The selection or use of alignment conditions that are individually tuned to a number of specific subject intervals to be sequenced allows optimization of speed, sensitivity, and specificity. The method is particularly effective when the alignment of reads for a relatively large number of diverse subject intervals are optimized.

In some instances, the method includes the use of an alignment method optimized for rearrangements in combination with other alignment methods optimized for subject intervals not associated with rearrangements.

In some instances, the methods disclosed herein further comprise selecting or using an alignment method for analyzing, e.g., aligning, a sequence read, wherein said alignment method is a function of, is selected responsive to, or is optimized for, one or more of: (i) tumor type, e.g., the tumor type in the sample; (ii) the location (e.g., a gene locus) of the subject interval being sequenced; (iii) the type of variant (e.g., a point mutation, insertion, deletion, substitution, copy number variation (CNV), rearrangement, or fusion) in the subject interval being sequenced; (iv) the site (e.g., nucleotide position) being analyzed; (v) the type of sample (e.g., a sample described herein); and/or (vi) adjacent sequence(s) in or near the subject interval being evaluated (e.g., according to the expected propensity thereof for misalignment of the subject interval due to, e.g., the presence of repeated sequences in or near the subject interval).

In some instances, the methods disclosed herein allow for the rapid and efficient alignment of troublesome reads, e.g., a read having a rearrangement. Thus, in some instances where a read for a subject interval comprises a nucleotide position with a rearrangement, e.g., a translocation, the method can comprise using an alignment method that is appropriately tuned and that includes: (i) selecting a rearrangement reference sequence for alignment with a read, wherein said rearrangement reference sequence aligns with a rearrangement (in some instances, the reference sequence is not identical to the genomic rearrangement); and (ii) comparing, e.g., aligning, a read with said rearrangement reference sequence.

In some instances, alternative methods may be used to align troublesome reads. These methods are particularly effective when the alignment of reads for a relatively large number of diverse subject intervals is optimized. By way of example, a method of analyzing a sample can comprise: (i) performing a comparison (e.g., an alignment comparison) of a read using a first set of parameters (e.g., using a first mapping algorithm, or by comparison with a first reference sequence), and determining if said read meets a first alignment criterion (e.g., the read can be aligned with said first reference sequence, e.g., with less than a specific number of mismatches); (ii) if said read fails to meet the first alignment criterion, performing a second alignment comparison using a second set of parameters, (e.g., using a second mapping algorithm, or by comparison with a second reference sequence); and (iii) optionally, determining if said read meets said second criterion (e.g., the read can be aligned with said second reference sequence, e.g., with less than a specific number of mismatches), wherein said second set of parameters comprises use of, e.g., said second reference sequence, which, compared with said first set of parameters, is more likely to result in an alignment with a read for a variant (e.g., a rearrangement, insertion, deletion, or translocation).

In some instances, the alignment of sequence reads in the disclosed methods may be combined with a mutation calling method as described elsewhere herein. As discussed herein, reduced sensitivity for detecting actual mutations may be addressed by evaluating the quality of alignments (manually or in an automated fashion) around expected mutation sites in the genes or genomic loci (e.g., gene loci) being analyzed. In some instances, the sites to be evaluated can be obtained from databases of the human genome (e.g., the HG19 human reference genome) or cancer mutations (e.g., COSMIC). Regions that are identified as problematic can be remedied with the use of an algorithm selected to give better performance in the relevant sequence context, e.g., by alignment optimization (or re-alignment) using slower, but more accurate alignment algorithms such as Smith-Waterman alignment. In cases where general alignment algorithms cannot remedy the problem, customized alignment approaches may be created by, e.g., adjustment of maximum difference mismatch penalty parameters for genes with a high likelihood of containing substitutions; adjusting specific mismatch penalty parameters based on specific mutation types that are common in certain tumor types (e.g. ChT in melanoma); or adjusting specific mismatch penalty parameters based on specific mutation types that are common in certain sample types (e.g. substitutions that are common in FFPE).

Reduced specificity (increased false positive rate) in the evaluated subject intervals due to misalignment can be assessed by manual or automated examination of all mutation calls in the sequencing data. Those regions found to be prone to spurious mutation calls due to misalignment can be subjected to alignment remedies as discussed above. In cases where no algorithmic remedy is found possible, “mutations” from the problem regions can be classified or screened out from the panel of targeted loci.

### Alignment of Methyl-Seq Sequence Reads

In some instances, the methods may include the use of an alignment method optimized for aligning sequence reads for DNA that has been converted using, e.g., a bisulfite reaction, to convert unmethylated cytosine residues to uracil (which is interpreted as a thymine in sequencing results). In some instances, sequence reads may be aligned to two genomes in silico, e.g., converted and unconverted versions of the reference genome, using such alignment tools. Methylation occurs primarily at CpG sites, but may also occur less frequently at non-CpG sites (e.g., CHG or CHH sites).

In some instances, the sequence read data may be obtained using a nucleic acid sequencing method comprising the use of a bisulfite- or enzymatic-conversion reaction (e.g., during library preparation) to convert non-methylated cytosine to uracil (see, e.g., Li, et al. (2011), “DNA Methylation Detection: Bisulfite Genomic Sequencing Analysis”, Methods Mol. Biol. 791:11-21).

In some instances, the sequence read data may be obtained using a nucleic acid sequencing method comprising the use of alternative chemical and/or enzymatic reactions (e.g., during library preparation) to convert non-methylated cytosine to uracil (or to convert methylated cytosine to dihydrouracil). For example, enzymatic deamination of non-methylated cytosine using APOBEC to form uracil can be performed using, e.g., the Enzymatic Methyl-seq Kit from New England BioLabs (Ipswich, MA) which uses prior treatment with ten-eleven translocation methylcytosine dioxygenase 2 (TET2) to oxidize 5-mC and 5-hmC, thereby providing greater protection of the methylated cytosine from deamination by APOBEC). Liu, et al. (2019) recently described a bisulfite-free and base-level-resolution sequencing-based method, TET-Assisted Pyridine borane Sequencing (TAPS), for detection of 5mC and 5hmC. The method combines ten-eleven translocation methylcytosine dioxygenase (TET)-mediated oxidation of 5mC and 5hmC to 5-carboxylcytosine (5caC) with pyridine borane reduction of 5caC to dihydrouracil (DHU). Subsequent PCR amplification converts DHU to thymine, thereby enabling conversion of methylated cytosines to thymine (Liu, et al. (2019), “Bisulfite-Free Direct Detection of 5-Methylcytosine and 5-Hydroxymethylcytosine at Base Resolution”, Nature Biotechnology, vol. 37, pp. 424-429).

In some instances, the sequence read data may be obtained using a nucleic acid sequencing method comprising the use of Methylated DNA Immunoprecipitation (MeDIP).

Examples of alignment tools optimized for aligning sequence reads for converted DNA include, but are not limited to, NovoAlign (Novocraft Technologies, Selangor, Malaysia), and the Bismark tool (Krueger, et al. (2011), “Bismark: A Flexible Aligner and Methylation Caller for Bisulfite-Seq Applications”, Bioinformatics 27(11):1571-1572).

### Mutation Calling

Base calling refers to the raw output of a sequencing device, e.g., the determined sequence of nucleotides in an oligonucleotide molecule. Mutation calling refers to the process of selecting a nucleotide value, e.g., A, G, T, or C, for a given nucleotide position being sequenced. Typically, the sequence reads (or base calling) for a position will provide more than one value, e.g., some reads will indicate a T and some will indicate a G. Mutation calling is the process of assigning a correct nucleotide value, e.g., one of those values, to the sequence. Although it is referred to as “mutation” calling, it can be applied to assign a nucleotide value to any nucleotide position, e.g., positions corresponding to mutant alleles, wild-type alleles, alleles that have not been characterized as either mutant or wild-type, or to positions not characterized by variability.

In some instances, the disclosed methods may comprise the use of customized or tuned mutation calling algorithms or parameters thereof to optimize performance when applied to sequencing data, particularly in methods that rely on massively parallel sequencing (MPS) of a large number of diverse genetic events at a large number of diverse genomic loci (e.g., gene loci, microsatellite regions, etc.) in samples, e.g., samples from a subject having cancer. Optimization of mutation calling is described in the art, e.g., as set out in International Patent Application Publication No. WO 2012/092426.

Methods for mutation calling can include one or more of the following: making independent calls based on the information at each position in the reference sequence (e.g., examining the sequence reads; examining the base calls and quality scores; calculating the probability of observed bases and quality scores given a potential genotype; and assigning genotypes (e.g., using Bayes' rule)); removing false positives (e.g., using depth thresholds to reject SNPs with read depth much lower or higher than expected; local realignment to remove false positives due to small indels); and performing linkage disequilibrium (LD)/imputation-based analysis to refine the calls.

Equations used to calculate the genotype likelihood associated with a specific genotype and position are described in, e.g., Li, H. and Durbin, R. Bioinformatics, 2010; 26(5): 589-95. The prior expectation for a particular mutation in a certain cancer type can be used when evaluating samples from that cancer type. Such likelihood can be derived from public databases of cancer mutations, e.g., Catalogue of Somatic Mutation in Cancer (COSMIC), HGMD (Human Gene Mutation Database), The SNP Consortium, Breast Cancer Mutation Data Base (BIC), and Breast Cancer Gene Database (BCGD).

Examples of LD/imputation based analysis are described in, e.g., Browning, B. L. and Yu, Z. Am. J. Hum. Genet. 2009, 85(6):847-61. Examples of low-coverage SNP calling methods are described in, e.g., Li, Y., et al., Annu. Rev. Genomics Hum. Genet. 2009, 10:387-406.

After alignment, detection of substitutions can be performed using a mutation calling method (e.g., a Bayesian mutation calling method) which is applied to each base in each of the subject intervals, e.g., exons of a gene or other locus to be evaluated, where presence of alternate alleles is observed. This method will compare the probability of observing the read data in the presence of a mutation with the probability of observing the read data in the presence of base-calling error alone. Mutations can be called if this comparison is sufficiently strongly supportive of the presence of a mutation.

An advantage of a Bayesian mutation detection approach is that the comparison of the probability of the presence of a mutation with the probability of base-calling error alone can be weighted by a prior expectation of the presence of a mutation at the site. If some reads of an alternate allele are observed at a frequently mutated site for the given cancer type, then presence of a mutation may be confidently called even if the amount of evidence of mutation does not meet the usual thresholds. This flexibility can then be used to increase detection sensitivity for even rarer mutations/lower purity samples, or to make the test more robust to decreases in read coverage. The likelihood of a random base-pair in the genome being mutated in cancer is ˜1e-6. The likelihood of specific mutations occurring at many sites in, for example, a typical multigenic cancer genome panel can be orders of magnitude higher. These likelihoods can be derived from public databases of cancer mutations (e.g., COSMIC).

Indel calling is a process of finding bases in the sequencing data that differ from the reference sequence by insertion or deletion, typically including an associated confidence score or statistical evidence metric. Methods of indel calling can include the steps of identifying candidate indels, calculating genotype likelihood through local re-alignment, and performing LD-based genotype inference and calling. Typically, a Bayesian approach is used to obtain potential indel candidates, and then these candidates are tested together with the reference sequence in a Bayesian framework.

Algorithms to generate candidate indels are described in, e.g., McKenna, A., et al., Genome Res. 2010; 20(9):1297-303; Ye, K., et al., Bioinformatics, 2009; 25(21):2865-71; Lunter, G., and Goodson, M., Genome Res. 2011; 21(6):936-9; and Li, H., et al. (2009), Bioinformatics 25(16):2078-9.

Methods for generating indel calls and individual-level genotype likelihoods include, e.g., the Dindel algorithm (Albers, C. A., et al., Genome Res. 2011; 21(6):961-73). For example, the Bayesian EM algorithm can be used to analyze the reads, make initial indel calls, and generate genotype likelihoods for each candidate indel, followed by imputation of genotypes using, e.g., QCALL (Le S. Q. and Durbin R. Genome Res. 2011; 21(6):952-60). Parameters, such as prior expectations of observing the indel can be adjusted (e.g., increased or decreased), based on the size or location of the indels.

Methods have been developed that address limited deviations from allele frequencies of 50% or 100% for the analysis of cancer DNA. (see, e.g., SNVMix-Bioinformatics. 2010 Mar. 15; 26(6): 730-736.) Methods disclosed herein, however, allow consideration of the possibility of the presence of a mutant allele at frequencies (or allele fractions) ranging from 1% to 100% (i.e., allele fractions ranging from 0.01 to 1.0), and especially at levels lower than 50%. This approach is particularly important for the detection of mutations in, for example, low-purity FFPE samples of natural (multi-clonal) tumor DNA.

In some instances, the mutation calling method used to analyze sequence reads is not individually customized or fine-tuned for detection of different mutations at different genomic loci. In some instances, different mutation calling methods are used that are individually customized or fine-tuned for at least a subset of the different mutations detected at different genomic loci. In some instances, different mutation calling methods are used that are individually customized or fine-tuned for each different mutant detected at each different genomic loci. The customization or tuning can be based on one or more of the factors described herein, e.g., the type of cancer in a sample, the gene or locus in which the subject interval to be sequenced is located, or the variant to be sequenced. This selection or use of mutation calling methods individually customized or fine-tuned for a number of subject intervals to be sequenced allows for optimization of speed, sensitivity and specificity of mutation calling.

In some instances, a nucleotide value is assigned for a nucleotide position in each of X unique subject intervals using a unique mutation calling method, and X is at least 2, at least 3, at least 4, at least 5, at least 10, at least 15, at least 20, at least 30, at least 40, at least 50, at least 60, at least 70, at least 80, at least 90, at least 100, at least 200, at least 300, at least 400, at least 500, at least 1000, at least 1500, at least 2000, at least 2500, at least 3000, at least 3500, at least 4000, at least 4500, at least 5000, or greater. The calling methods can differ, and thereby be unique, e.g., by relying on different Bayesian prior values.

In some instances, assigning said nucleotide value is a function of a value which is or represents the prior (e.g., literature) expectation of observing a read showing a variant, e.g., a mutation, at said nucleotide position in a tumor of type.

In some instances, the method comprises assigning a nucleotide value (e.g., calling a mutation) for at least 10, 20, 40, 50, 60, 70, 80, 90, 100, 200, 300, 400, 500, 600, 700, 800, 900, or 1,000 nucleotide positions, wherein each assignment is a function of a unique value (as opposed to the value for the other assignments) which is or represents the prior (e.g., literature) expectation of observing a read showing a variant, e.g., a mutation, at said nucleotide position in a tumor of type.

In some instances, assigning said nucleotide value is a function of a set of values which represent the probabilities of observing a read showing said variant at said nucleotide position if the variant is present in the sample at a specified frequency (e.g., 1%, 5%, 10%, etc.) and/or if the variant is absent (e.g., observed in the reads due to base-calling error alone).

In some instances, the mutation calling methods described herein can include the following: (a) acquiring, for a nucleotide position in each of said X subject intervals: (i) a first value which is or represents the prior (e.g., literature) expectation of observing a read showing a variant, e.g., a mutation, at said nucleotide position in a tumor of type X; and (ii) a second set of values which represent the probabilities of observing a read showing said variant at said nucleotide position if the variant is present in the sample at a frequency (e.g., 1%, 5%, 10%, etc.) and/or if the variant is absent (e.g., observed in the reads due to base-calling error alone); and (b) responsive to said values, assigning a nucleotide value (e.g., calling a mutation) from said reads for each of said nucleotide positions by weighing, e.g., by a Bayesian method described herein, the comparison among the values in the second set using the first value (e.g., computing the posterior probability of the presence of a mutation), thereby analyzing said sample.

Additional description of exemplary nucleic acid sequencing methods, mutation calling methods, and methods for analysis of genetic variants is provided in, e.g., U.S. Pat. Nos. 9,340,830, 9,792,403, 11,136,619, 11,118,213, and International Patent Application Publication No. WO 2020/236941, the entire contents of each of which is incorporated herein by reference.

### Methylation Status Calling

In some instances, the methods described herein may comprise the use of a methylation status calling method, e.g., to call the methylation status of the CpG sites based on the sequence reads and fragments (complementary pairs of forward and reverse sequence reads) derived from DNA that has been subjected to a chemical or enzymatic conversion reaction, e.g., to convert unmethylated cytosine residues to uracil (which is interpreted as a thymine in sequencing results). Examples of such methylation status calling tools include, but are not limited to, the Bismark tool (Krueger, et al. (2011), “Bismark: A Flexible Aligner and Methylation Caller for Bisulfite-Seq Applications”, Bioinformatics 27(11):1571-1572), TARGOMICS (Garinet, et al. (2017), “Calling Chromosome Alterations, DNA Methylation Statuses, and Mutations in Tumors by Simple Targeted Next-Generation Sequencing—A Solution for Transferring Integrated Pangenomic Studies into Routine Practice?”, J. Molecular Diagnostics 19(5):776-787), Bicycle (Grana, et al. (2018) “Bicycle: A Bioinformatics Pipeline to Analyze Bisulfite Sequencing Data”, Bioinformatics 34(8):1414-5), SMAP (Gao, et al. (2015), “SMAP: A Streamlined Methylation Analysis Pipeline for Bisulfite Sequencing”, Gigascience 4:29), and MeDUSA (Wilson, et al. (2016), “Computational Analysis and Integration of MeDIP-Seq Methylome Data”, in: Kulski J K, editor, Next Generation Sequencing: Advances, Applications and Challenges. Rijeka: InTech, p. 153-69). See also, Rauluseviciute, et al. (2019), “DNA Methylation Data by Sequencing: Experimental Approaches and Recommendations for Tools and Pipelines for Data Analysis”, Clinical Epigenetics 11:193.

### Systems

Also disclosed herein are systems designed to implement any of the disclosed methods for analyzing or assigning a disease label in a sample from a subject. The systems may comprise, e.g., one or more processors, and a memory unit communicatively coupled to the one or more processors and configured to store instructions that, when executed by the one or more processors, cause the system to: receive, by one or more processors, test sample characterization data comprising genomic alteration statuses for a test sample, wherein the test sample has a predetermined disease label; receive, by the one or more processors, for database samples, database characterization data, wherein at least one database sample in the database samples has a predetermined disease label; determine, by the one or more processors, similarity scores for the database samples, wherein the similarity scores indicate similarities between the test sample characterization data and the database characterization data; rank, by the one or more processors, the database samples based on the similarity scores, to generate ranked database samples; select, by the one or more processors, from the ranked database samples, a subset of database samples most similar to the test sample; determine, by the one or more processors, an enrichment score for the predetermined disease label based on the subset of database samples and the database samples; and confirm or reject, by the one or more processors, the predetermined disease label for the test sample based on the enrichment score.

In some instances, the disclosed systems may further comprise a sequencer, e.g., a next generation sequencer (also referred to as a massively parallel sequencer). Examples of next generation (or massively parallel) sequencing platforms include, but are not limited to, Roche/454's Genome Sequencer (GS) FLX system, Illumina/Solexa's Genome Analyzer (GA), Illumina's HiSeq® 2500, HiSeq® 3000, HiSeq® 4000 and NovaSeq® 6000 sequencing systems, Life/APG's Support Oligonucleotide Ligation Detection (SOLiD) system, Polonator's G.007 system, Helicos BioSciences' HeliScope Gene Sequencing system, ThermoFisher Scientific's Ion Torrent Genexus system, or Pacific Biosciences' PacBio® RS system.

In some instances, the disclosed systems may be used for analyzing or assigning a disease label for any of a variety of samples as described herein (e.g., a tissue sample, biopsy sample, hematological sample, or liquid biopsy sample derived from the subject).

In some instances, the plurality of gene loci for which sequencing data is processed to analyze or assign a disease label may comprise at least 1, 2, 3, 4, 5, 6, 7, 8, 9, 10, or more than 10 gene loci. The resulting analyzed or assigned disease label can be used to treat the disease label, such as cancer.

In some instance, the nucleic acid sequence data is acquired using a next generation sequencing technique (also referred to as a massively parallel sequencing technique) having a read-length of less than 400 bases, less than 300 bases, less than 200 bases, less than 150 bases, less than 100 bases, less than 90 bases, less than 80 bases, less than 70 bases, less than 60 bases, less than 50 bases, less than 40 bases, or less than 30 bases.

In some instances, the analyzing or assigning a disease label can be used to select, initiate, adjust, or terminate a treatment for cancer in the subject (e.g., a patient) from which the sample was derived, as described elsewhere herein.

In some instances, the disclosed systems may further comprise sample processing and library preparation workstations, microplate-handling robotics, fluid dispensing systems, temperature control modules, environmental control chambers, additional data storage modules, data communication modules (e.g., Bluetooth®, WiFi, intranet, or internet communication hardware and associated software), display modules, one or more local and/or cloud-based software packages (e.g., instrument/system control software packages, sequencing data analysis software packages), etc., or any combination thereof. In some instances, the systems may comprise, or be part of, a computer system or computer network as described elsewhere herein.

### Computer Systems and Networks

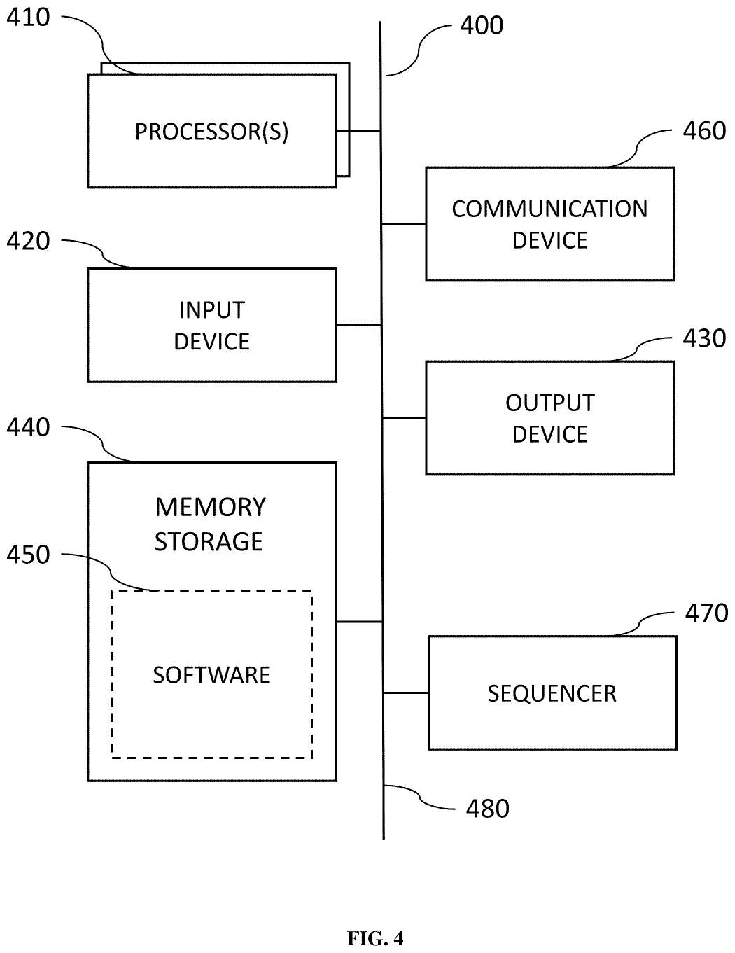

FIG. 4 illustrates an example of a computing device or system in accordance with one embodiment. Device 400 can be a host computer connected to a network. Device 900 can be a client computer or a server. As shown in FIG. 4, device 400 can be any suitable type of microprocessor-based device, such as a personal computer, workstation, server or handheld computing device (portable electronic device) such as a phone or tablet. The device can include, for example, one or more processor(s) 410, input devices 420, output devices 430, memory or storage devices 440, communication devices 460, and nucleic acid sequencers 470. Software 450 residing in memory or storage device 440 may comprise, e.g., an operating system as well as software for executing the methods described herein. Input device 420 and output device 430 can generally correspond to those described herein, and can either be connectable or integrated with the computer.

Input device 420 can be any suitable device that provides input, such as a touch screen, keyboard or keypad, mouse, or voice-recognition device. Output device 430 can be any suitable device that provides output, such as a touch screen, haptics device, or speaker.

Storage 440 can be any suitable device that provides storage (e.g., an electrical, magnetic or optical memory including a RAM (volatile and non-volatile), cache, hard drive, or removable storage disk). Communication device 460 can include any suitable device capable of transmitting and receiving signals over a network, such as a network interface chip or device. The components of the computer can be connected in any suitable manner, such as via a wired media (e.g., a physical system bus 480, Ethernet connection, or any other wire transfer technology) or wirelessly (e.g., Bluetooth®, Wi-Fi®, or any other wireless technology).

Software module 450, which can be stored as executable instructions in storage 440 and executed by processor(s) 410, can include, for example, an operating system and/or the processes that embody the functionality of the methods of the present disclosure (e.g., as embodied in the devices as described herein).

Software module 450 can also be stored and/or transported within any non-transitory computer-readable storage medium for use by or in connection with an instruction execution system, apparatus, or device, such as those described herein, that can fetch instructions associated with the software from the instruction execution system, apparatus, or device and execute the instructions. In the context of this disclosure, a computer-readable storage medium can be any medium, such as storage 440, that can contain or store processes for use by or in connection with an instruction execution system, apparatus, or device. Examples of computer-readable storage media may include memory units like hard drives, flash drives and distribute modules that operate as a single functional unit. Also, various processes described herein may be embodied as modules configured to operate in accordance with the embodiments and techniques described above. Further, while processes may be shown and/or described separately, those skilled in the art will appreciate that the above processes may be routines or modules within other processes.

Software module 450 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

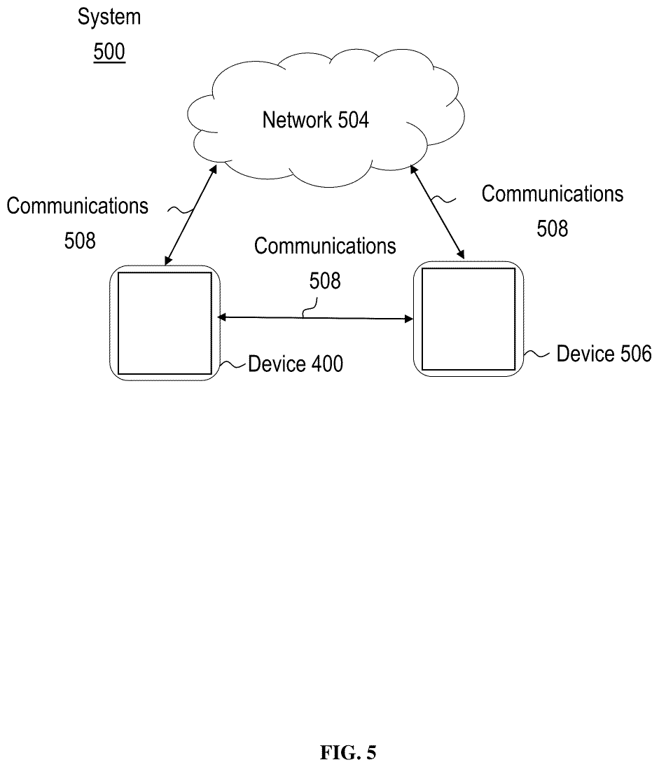

Device 400 may be connected to a network (e.g., network 504, as shown in FIG. 5 and/or described below), which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 400 can be implemented using any operating system, e.g., an operating system suitable for operating on the network. Software module 950 can be written in any suitable programming language, such as C, C++, Java or Python. In various embodiments, application software embodying the functionality of the present disclosure can be deployed in different configurations, such as in a client/server arrangement or through a Web browser as a Web-based application or Web service, for example. In some embodiments, the operating system is executed by one or more processors, e.g., processor(s) 410.

Device 400 can further include a sequencer 470, which can be any suitable nucleic acid sequencing instrument.

FIG. 5 illustrates an example of a computing system in accordance with one embodiment. In system 500, device 400 (e.g., as described above and illustrated in FIG. 4) is connected to network 1004, which is also connected to device 506. In some embodiments, device 506 is a sequencer. Exemplary sequencers can include, without limitation, Roche/454's Genome Sequencer (GS) FLX System, Illumina/Solexa's Genome Analyzer (GA), Illumina's HiSeq® 2500, HiSeq® 3000, HiSeq® 4000 and NovaSeq® 6000 Sequencing Systems, Life/APG's Support Oligonucleotide Ligation Detection (SOLiD) system, Polonator's G.007 system, Helicos BioSciences' HeliScope Gene Sequencing system, or Pacific Biosciences' PacBio® RS system.

Devices 400 and 506 may communicate, e.g., using suitable communication interfaces via network 1004, such as a Local Area Network (LAN), Virtual Private Network (VPN), or the Internet. In some embodiments, network 504 can be, for example, the Internet, an intranet, a virtual private network, a cloud network, a wired network, or a wireless network. Devices 400 and 506 may communicate, in part or in whole, via wireless or hardwired communications, such as Ethernet, IEEE 802.11b wireless, or the like. Additionally, devices 400 and 506 may communicate, e.g., using suitable communication interfaces, via a second network, such as a mobile/cellular network. Communication between devices 400 and 506 may further include or communicate with various servers such as a mail server, mobile server, media server, telephone server, and the like. In some embodiments, Devices 400 and 506 can communicate directly (instead of, or in addition to, communicating via network 504), e.g., via wireless or hardwired communications, such as Ethernet, IEEE 802.11b wireless, or the like. In some embodiments, devices 400 and 506 communicate via communications 508, which can be a direct connection or can occur via a network (e.g., network 504).

One or all of devices 400 and 506 generally include logic (e.g., http web server logic) or are programmed to format data, accessed from local or remote databases or other sources of data and content, for providing and/or receiving information via network 504 according to various examples described herein.

### EXEMPLARY EMBODIMENTS

The following embodiments are exemplary and are not intended to limit the scope of any claims.

Embodiment 1. A method comprising:
providing nucleic acid molecules obtained from a test sample from a subject;
ligating adapters onto the nucleic acid molecules;
amplifying the ligated nucleic acid molecules;
capturing the amplified nucleic acid molecules;
sequencing, by a sequencer, the captured nucleic acid molecules to obtain sequence reads that represent the captured nucleic acid molecules;
receiving, by one or more processors, sequence read data for the sequence reads;
determining genomic alteration statuses for the test sample from the sequence read data;
receiving, by the one or more processors, test sample characterization data comprising the genomic alteration statuses for the test sample, wherein the test sample has a predetermined disease label;
receiving, by the one or more processors, for database samples, database characterization data, wherein at least one database sample in the database samples has a predetermined database disease label;
determining, by the one or more processors, similarity scores for the database samples, wherein the similarity scores indicate similarities between the test sample characterization data, and the database characterization data;
ranking, by the one or more processors, the database samples based on the similarity scores, to generate ranked database samples;
selecting, by the one or more processors, from the ranked database samples, a subset of database samples most similar to the test sample;
determining, by the one or more processors, an enrichment score for the predetermined disease label based on the subset of database samples and the database samples; and
confirming or rejecting, by the one or more processors, the predetermined disease label for the test sample based on the enrichment score.

Embodiment 2. A method for detecting a disease type comprising:
receiving, by one or more processors, test sample characterization data comprising genomic alteration statuses for a test sample, wherein the test sample has a predetermined disease label;
receiving, by the one or more processors, for database samples, database characterization data, wherein at least one database sample in the database samples has a predetermined disease label;
determining, by the one or more processors, similarity scores for the database samples, wherein the similarity scores indicate similarities between the test sample characterization data and the database characterization data;
ranking, by the one or more processors, the database samples based on the similarity scores, to generate ranked database samples;
selecting, by the one or more processors, from the ranked database samples, a subset of database samples most similar to the test sample;
determining, by the one or more processors, an enrichment score for the predetermined disease label based on the subset of database samples and the database samples; and
confirming or rejecting, by the one or more processors, the predetermined disease label for the test sample based on the enrichment score.

Embodiment 3. The method of embodiment 1 or 2, further comprising:
excluding, by the one or more processors, for the test sample, a genomic alteration status from the test sample characterization data, to generate reduced test sample characterization data;
excluding, by the one or more processors, for the database samples, the genomic alteration status from the database characterization data, to generate reduced database characterization data;
determining, by the one or more processors, second similarity scores, wherein the second similarity scores indicate similarities between the reduced test sample characterization data, and for a corresponding database sample, the reduced database characterization data;
ranking, by the one or more processors, the database samples based on the second similarity scores, to generate second ranked database samples;
selecting, by the one or more processors, from the second ranked database samples, a second subset of database samples most similar to the test sample;
determining, by the one or more processors, a second enrichment score for the predetermined disease label based on the second subset of database samples and the database samples; and
confirming or rejecting, by the one or more processors, the predetermined disease label for the test sample based on the enrichment score and the second enrichment score.

Embodiment 4. The method of any of embodiments 1-3, further comprising:
determining, by the one or more processors, one or more alternate enrichment scores for one or more alternate disease labels based on the subset of database samples and the database samples;
rejecting, by the one or more processors, the predetermined disease label; and
assigning, by the one or more processors, an alternate disease label from the one or more alternate disease labels for the test sample based on the enrichment score.

Embodiment 5. The method of embodiment 4, wherein the alternate disease label is a type of cancer.

Embodiment 6. The method of embodiment 5, wherein the alternate disease label indicates that the disease is unknown.

Embodiment 7. The method of embodiment 6, wherein the disease is unknown, when the number of database samples in the ranked database samples is less than or equal to a predetermined number of database samples.

Embodiment 8. The method of any of embodiments 4-7, wherein the alternate disease label is a cancer of unknown primary.

Embodiment 9. The method of any of embodiments 1-8, wherein the subject is suspected of having or is determined to have cancer.

Embodiment 10. The method of embodiment 9, wherein the cancer is a B cell cancer (multiple myeloma), a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.

Embodiment 11. The method of embodiment 9, wherein the cancer comprises acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR/MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma, Hodgkin lymphoma, juvenile idiopathic arthritis, lupus erythematosus, a mantle cell lymphoma, medullary thyroid cancer, melanoma, a melanoma with a BRAF V600 mutation, a melanoma with a BRAF V600E or V600K mutation, Merkel cell carcinoma, multicentric Castleman's disease, multiple hematologic malignancies including Philadelphia chromosome-positive ALL and CML, multiple myeloma, myelofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROS1 gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, rheumatoid arthritis, a small lymphocytic lymphoma, a soft tissue sarcoma, a solid tumor (MSI-H/dMMR), a squamous cell cancer of the head and neck, a squamous non-small cell lung cancer, thyroid cancer, a thyroid carcinoma, urothelial cancer, a urothelial carcinoma, or Waldenstrom's macroglobulinemia.

Embodiment 12. The method of embodiment 9, further comprising treating the subject with an anti-cancer therapy.

Embodiment 13. The method of embodiment 12, wherein the anti-cancer therapy comprises a targeted anti-cancer therapy.

Embodiment 14. The method of embodiment 13, wherein the targeted anti-cancer therapy comprises abemaciclib (Verzenio), abiraterone acetate (Zytiga), acalabrutinib (Calquence), ado-trastuzumab emtansine (Kadcyla), afatinib dimaleate (Gilotrif), aldesleukin (Proleukin), alectinib (Alecensa), alemtuzumab (Campath), alitretinoin (Panretin), alpelisib (Piqray), amivantamab-vmjw (Rybrevant), anastrozole (Arimidex), apalutamide (Erleada), asciminib hydrochloride (Scemblix), atezolizumab (Tecentriq), avapritinib (Ayvakit), avelumab (Bavencio), axicabtagene ciloleucel (Yescarta), axitinib (Inlyta), belantamab mafodotin-blmf (Blenrep), belimumab (Benlysta), belinostat (Beleodaq), belzutifan (Welireg), bevacizumab (Avastin), bexarotene (Targretin), binimetinib (Mektovi), blinatumomab (Blincyto), bortezomib (Velcade), bosutinib (Bosulif), brentuximab vedotin (Adcetris), brexucabtagene autoleucel (Tecartus), brigatinib (Alunbrig), cabazitaxel (Jevtana), cabozantinib (Cabometyx), cabozantinib (Cabometyx, Cometriq), canakinumab (Ilaris), capmatinib hydrochloride (Tabrecta), capivasertib, carfilzomib (Kyprolis), cemiplimab-rwlc (Libtayo), ceritinib (LDK378/Zykadia), cetuximab (Erbitux), cobimetinib (Cotellic), copanlisib hydrochloride (Aligopa), crizotinib (Xalkori), dabrafenib (Tafinlar), dacomitinib (Vizimpro), daratumumab (Darzalex), daratumumab and hyaluronidase-fihj (Darzalex Faspro), darolutamide (Nubega), dasatinib (Sprycel), denileukin diftitox (Ontak), denosumab (Xgeva), dinutuximab (Unituxin), dostarlimab-gxly (Jemperli), durvalumab (Imfinzi), duvelisib (Copiktra), elotuzumab (Empliciti), enasidenib mesylate (Idhifa), encorafenib (Braftovi), enfortumab vedotin-ejfv (Padcev), entrectinib (Rozlytrek), enzalutamide (Xtandi), erdafitinib (Balversa), erlotinib (Tarceva), everolimus (Afinitor), exemestane (Aromasin), fam-trastuzumab deruxtecan-nxki (Enhertu), fedratinib hydrochloride (Inrebic), fulvestrant (Faslodex), gefitinib (Iressa), gemtuzumab ozogamicin (Mylotarg), gilteritinib (Xospata), glasdegib maleate (Daurismo), hyaluronidase-zzxf (Phesgo), ibrutinib (Imbruvica), ibritumomab tiuxetan (Zevalin), idecabtagene vicleucel (Abecma), idelalisib (Zydelig), imatinib mesylate (Gleevec), infigratinib phosphate (Truseltiq), inotuzumab ozogamicin (Besponsa), iobenguane I131 (Azedra), ipilimumab (Yervoy), isatuximab-irfc (Sarclisa), ivosidenib (Tibsovo), ixazomib citrate (Ninlaro), lanreotide acetate (Somatuline Depot), lapatinib (Tykerb), larotrectinib sulfate (Vitrakvi), lenvatinib mesylate (Lenvima), letrozole (Femara), lisocabtagene maraleucel (Breyanzi), loncastuximab tesirine-lpyl (Zynlonta), lorlatinib (Lorbrena), lutetium Lu 177-dotatate (Lutathera), margetuximab-cmkb (Margenza), midostaurin (Rydapt), mobocertinib succinate (Exkivity), mogamulizumab-kpkc (Poteligeo), moxetumomab pasudotox-tdfk (Lumoxiti), naxitamab-gqgk (Danyelza), necitumumab (Portrazza), neratinib maleate (Nerlynx), nilotinib (Tasigna), niraparib tosylate monohydrate (Zejula), nivolumab (Opdivo), obinutuzumab (Gazyva), ofatumumab (Arzerra), olaparib (Lynparza), olaratumab (Lartruvo), osimertinib (Tagrisso), palbociclib (Ibrance), panitumumab (Vectibix), panobinostat (Farydak), pazopanib (Votrient), pembrolizumab (Keytruda), pemigatinib (Pemazyre), pertuzumab (Perjeta), pexidartinib hydrochloride (Turalio), polatuzumab vedotin-piiq (Polivy), ponatinib hydrochloride (Iclusig), pralatrexate (Folotyn), pralsetinib (Gavreto), radium 223 dichloride (Xofigo), ramucirumab (Cyramza), regorafenib (Stivarga), ribociclib (Kisqali), ripretinib (Qinlock), rituximab (Rituxan), rituximab and hyaluronidase human (Rituxan Hycela), romidepsin (Istodax), rucaparib camsylate (Rubraca), ruxolitinib phosphate (Jakafi), sacituzumab govitecan-hziy (Trodelvy), seliciclib, selinexor (Xpovio), selpercatinib (Retevmo), selumetinib sulfate (Koselugo), siltuximab (Sylvant), sipuleucel-T (Provenge), sirolimus protein-bound particles (Fyarro), sonidegib (Odomzo), sorafenib (Nexavar), sotorasib (Lumakras), sunitinib (Sutent), tafasitamab-cxix (Monjuvi), tagraxofusp-erzs (Elzonris), talazoparib tosylate (Talzenna), tamoxifen (Nolvadex), tazemetostat hydrobromide (Tazverik), tebentafusp-tebn (Kimmtrak), temsirolimus (Torisel), tepotinib hydrochloride (Tepmetko), tisagenlecleucel (Kymriah), tisotumab vedotin-tftv (Tivdak), tocilizumab (Actemra), tofacitinib (Xeljanz), tositumomab (Bexxar), trametinib (Mekinist), trastuzumab (Herceptin), tretinoin (Vesanoid), tivozanib hydrochloride (Fotivda), toremifene (Fareston), tucatinib (Tukysa), umbralisib tosylate (Ukoniq), vandetanib (Caprelsa), vemurafenib (Zelboraf), venetoclax (Venclexta), vismodegib (Erivedge), vorinostat (Zolinza), zanubrutinib (Brukinsa), ziv-aflibercept (Zaltrap), or any combination thereof.

Embodiment 15. The method of any of embodiments 1-14, further comprising obtaining the test sample from the subject.

Embodiment 16. The method of any of embodiments 1-15, wherein the test sample comprises a tissue biopsy sample, a liquid biopsy sample, or a normal control.

Embodiment 17. The method of embodiment 16, wherein the test sample is a liquid biopsy sample and comprises blood, plasma, cerebrospinal fluid, sputum, stool, urine, or saliva.

Embodiment 18. The method of embodiment 16, wherein the test sample is a liquid biopsy sample and comprises circulating tumor cells (CTCs).

Embodiment 19. The method of embodiment 16, wherein the test sample is a liquid biopsy sample and comprises cell-free DNA (cfDNA), circulating tumor DNA (ctDNA), or any combination thereof.

Embodiment 20. The method of any of embodiments 1-19, wherein the plurality of nucleic acid molecules comprises a mixture of tumor nucleic acid molecules and non-tumor nucleic acid molecules.

Embodiment 21. The method of embodiment 20, wherein the tumor nucleic acid molecules are derived from a tumor portion of a heterogeneous tissue biopsy sample, and the non-tumor nucleic acid molecules are derived from a normal portion of the heterogeneous tissue biopsy sample.

Embodiment 22. The method of embodiment 20, wherein the sample comprises a liquid biopsy sample, and wherein the tumor nucleic acid molecules are derived from a circulating tumor DNA (ctDNA) fraction of the liquid biopsy sample, and the non-tumor nucleic acid molecules are derived from a non-tumor, cell-free DNA (cfDNA) fraction of the liquid biopsy sample.

Embodiment 23. The method of any of embodiments 1-22, wherein the one or more adapters comprise amplification primers, flow cell adaptor sequences, substrate adapter sequences, or sample index sequences.

Embodiment 24. The method of any of embodiments 1-23, wherein the captured nucleic acid molecules are captured from the amplified nucleic acid molecules by hybridization to one or more bait molecules.

Embodiment 25. The method of embodiment 24, wherein the one or more bait molecules comprise one or more nucleic acid molecules, each comprising a region that is complementary to a region of a captured nucleic acid molecule.

Embodiment 26. The method of any of embodiments 1-25, wherein amplifying nucleic acid molecules comprises performing a polymerase chain reaction (PCR) amplification technique, a non-PCR amplification technique, or an isothermal amplification technique.

Embodiment 27. The method of any of embodiments 1-26, wherein the sequencing comprises use of a massively parallel sequencing (MPS) technique, whole genome sequencing (WGS), whole exome sequencing, targeted sequencing, direct sequencing, or Sanger sequencing technique.

Embodiment 28. The method of embodiment 27, wherein the sequencing comprises massively parallel sequencing, and the massively parallel sequencing technique comprises next generation sequencing (NGS).

Embodiment 29. The method of any of embodiments 1-28, wherein the sequencer comprises a next generation sequencer.

Embodiment 30. The method of any of embodiments 1-29, wherein one or more of the plurality of sequencing reads overlap one or more gene loci within one or more subgenomic intervals in the sample.

Embodiment 31. The method of embodiment 30, wherein the one or more gene loci comprises between 10 and 20 loci, between 10 and 40 loci, between 10 and 60 loci, between 10 and 80 loci, between 10 and 100 loci, between 10 and 150 loci, between 10 and 200 loci, between 10 and 250 loci, between 10 and 300 loci, between 10 and 350 loci, between 10 and 400 loci, between 10 and 450 loci, between 10 and 500 loci, between 20 and 40 loci, between 20 and 60 loci, between 20 and 80 loci, between 20 and 100 loci, between 20 and 150 loci, between 20 and 200 loci, between 20 and 250 loci, between 20 and 300 loci, between 20 and 350 loci, between 20 and 400 loci, between 20 and 500 loci, between 40 and 60 loci, between 40 and 80 loci, between 40 and 100 loci, between 40 and 150 loci, between 40 and 200 loci, between 40 and 250 loci, between 40 and 300 loci, between 40 and 350 loci, between 40 and 400 loci, between 40 and 500 loci, between 60 and 80 loci, between 60 and 100 loci, between 60 and 150 loci, between 60 and 200 loci, between 60 and 250 loci, between 60 and 300 loci, between 60 and 350 loci, between 60 and 400 loci, between 60 and 500 loci, between 80 and 100 loci, between 80 and 150 loci, between 80 and 200 loci, between 80 and 250 loci, between 80 and 300 loci, between 80 and 350 loci, between 80 and 400 loci, between 80 and 500 loci, between 100 and 150 loci, between 100 and 200 loci, between 100 and 250 loci, between 100 and 300 loci, between 100 and 350 loci, between 100 and 400 loci, between 100 and 500 loci, between 150 and 200 loci, between 150 and 250 loci, between 150 and 300 loci, between 150 and 350 loci, between 150 and 400 loci, between 150 and 500 loci, between 200 and 250 loci, between 200 and 300 loci, between 200 and 350 loci, between 200 and 400 loci, between 200 and 500 loci, between 250 and 300 loci, between 250 and 350 loci, between 250 and 400 loci, between 250 and 500 loci, between 300 and 350 loci, between 300 and 400 loci, between 300 and 500 loci, between 350 and 400 loci, between 350 and 500 loci, or between 400 and 500 loci.

Embodiment 32. The method of embodiment 30 or 31, wherein the one or more gene loci comprise ABL1, ACVR1B, AKT1, AKT2, AKT3, ALK, ALOX12B, AMER1, APC, AR, ARAF, ARFRP1, ARID1A, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKN1A, CDKN1B, CDKN2A, CDKN2B, CDKN2C, CEBPA, CHEK1, CHEK2, CIC, CREBBP, CRKL, CSF1R, CSF3R, CTCF, CTNNA1, CTNNB1, CUL3, CUL4A, CXCR4, CYP17A1, DAXX, DDR1, DDR2, DIS3, DNMT3A, DOT1L, EED, EGFR, EMSY (C11orf30), EP300, EPHA3, EPHB1, EPHB4, ERBB2, ERBB3, ERBB4, ERCC4, ERG, ERRFI1, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNF1A, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MST1R, MTAP, MTOR, MUTYH, MYB, MYC, MYCL, MYCN, MYD88, NBN, NF1, NF2, NFE2L2, NFKBIA, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCD1LG2, PDGFRA, PDGFRB, PDK1, PIK3C2B, PIK3C2G, PIK3CA, PIK3CB, PIK3R1, PIM1, PMS2, POLD1, POLE, PPARG, PPP2R1A, PPP2R2A, PRDM1, PRKAR1A, PRKCI, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSC1L1, WT1, XPO1, XRCC2, ZNF217, ZNF703, or any combination thereof.

Embodiment 33. The method of embodiment 31 or 32, wherein the one or more gene loci comprise ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CS1, CTLA-4, dMMR, EGFR, ERBB1, ERBB2, FGFR1-3, FLT3, GD2, HDAC, HER1, HER2, HR, IDH2, IL-10, IL-6, IL-6R, JAK1, JAK2, JAK3, KIT, KRAS, MEK, MET, MSI-H, mTOR, PARP, PD-1, PDGFR, PDGFRα, PDGFRβ, PD-L1, PI3Kδ, PIGF, PTCH, RAF, RANKL, RET, ROS1, SLAMF7, VEGF, VEGFA, VEGFB, or any combination thereof.

Embodiment 34. The method of any of embodiments 4-33, further comprising generating, by the one or more processors, a report indicating the predetermined disease label or assigning the alternate disease label to the test sample.

Embodiment 35. The method of embodiment 34, further comprising transmitting the report to a healthcare provider.

Embodiment 36. The method of embodiment 35, wherein the report is transmitted via a computer network or a peer-to-peer connection.

Embodiment 37. The method of any of embodiments 1-36, wherein selecting from the genomic alteration statuses comprise pathogenic genomic alteration statuses.

Embodiment 38. The method of any of embodiments 1-37, wherein one or more of the determined similarity scores or one or more of the determined second similarity scores increase by a predetermined pathogenic short variant scoring value, when the test sample and the corresponding database sample share a pathogenic short variant affecting a same gene.

Embodiment 39. The method of any of embodiments 1-38, wherein one or more of the determined similarity scores or one or more of the determined second similarity scores increase by a predetermined same pathogenic effect scoring value, when the test sample and the corresponding database sample share a pathogenic short variant affecting a same gene with identical protein effects.

Embodiment 40. The method of any of embodiments 1-39, wherein one or more of the determined similarity scores or one or more of the determined second similarity scores increase by a predetermined pathogenic copy number amplification scoring value, when the test sample and the corresponding database sample share a pathogenic copy number amplification occurring on a same amplicon segment.

Embodiment 41. The method of any of embodiments 1-40, wherein one or more of the determined similarity scores or one or more of the determined second similarity scores increases by a predetermined pathogenic copy number deletion scoring value, when the test sample and the corresponding database sample share a pathogenic copy number deletion occurring on a same commonly deleted segment.

Embodiment 42. The method of any of embodiments 1-41, wherein one or more of the determined similarity scores or one or more of the determined second similarity scores increase by a predetermined pathogenic rearrangement scoring value, when the test sample and the corresponding database sample share a same two gene partners in a pathogenic rearrangement.

Embodiment 43. The method of any of embodiments 1-42, wherein one or more of the determined similarity scores or one or more of the determined second similarity scores increase by a predetermined same gene partner pathogenic rearrangement scoring value, when the test sample and the corresponding database sample share a same one gene partner in a pathogenic rearrangement.

Embodiment 44. The method of any of embodiments 1-43, wherein one or more of the determined similarity scores or one or more of the determined second similarity scores decrease by a predetermined non-common genomic alteration status scoring value, when the test sample and the corresponding database sample do not share a same genomic alteration status from the genomic alteration statuses.

Embodiment 45. The method of any of embodiments 1-44, wherein one or more of the determined similarity scores or one or more of the determined second similarity scores increase by a predetermined tumor mutational burden (TMB) scoring value, when the test sample and the corresponding database sample each have a TMB score above a predetermined TMB score threshold.

Embodiment 46. The method of any of embodiments 1-45, wherein one or more of the determined similarity scores or one or more of the determined second similarity scores increase by a predetermined dominant mutational signature scoring value, when the test sample and the one database sample share a dominant mutational signature.

Embodiment 47. The method of embodiment 46, wherein the dominant mutational signature is associated with exposure to an alkylating agent, tobacco, or ultraviolet light, or an altered activity of APOBEC, a mutation in one or more mismatch repair pathway genes, a mutation in a POLE gene, or a mutation in BRCA1 or BRCA2.

Embodiment 48. The method of any of embodiments 1-47, wherein one or more of the determined similarity scores or one or more of the determined second similarity scores increase by a predetermined high TMB and dominant mutational signature scoring value, when the test sample and the corresponding database sample share both the high TMB score and the dominant mutational signature.

Embodiment 49. The method of any of embodiments 1-48, wherein the one or more determined similarity scores or the one or more determined second similarity scores increase by a predetermined copy number signature scoring value, when the test sample and the corresponding database sample share a copy number signature.

Embodiment 50. The method of any of embodiments 1-49, wherein the one or more determined similarity scores or the one or more determined second similarity scores increase by a predetermined aneuploidy feature scoring value, when the test sample and the corresponding database sample share a common aneuploidy feature.

Embodiment 51. The method of any of embodiments 1-50, wherein all the database samples from the ranked database samples have the determined similarity score or the determined second similarity score of at least a minimum score.

Embodiment 52. The method of any of embodiments 1-51, wherein database samples corresponding to a predetermined number of most similar database samples are used for determining the enrichment score or the second enrichment score.

Embodiment 53. The method of any of embodiments 1-52, wherein the enrichment score or the second enrichment score is an odds ratio.

Embodiment 54. The method of any of embodiments 1-53, wherein the odds ratio is determined by Fisher's exact test.

Embodiment 55. The method of any of embodiments 1-54, wherein the enrichment score or the second enrichment score is a U score from a Mann Whitney U-test.

Embodiment 56. The method of any of embodiments 1-55, further comprising determining a confidence value indicating whether the predetermined disease label is correctly confirmed or rejected.

Embodiment 57. The method of embodiment 56, wherein the confidence value is a p-value determined by the Fisher's exact test.

Embodiment 58. The method of any of embodiments 1-57, wherein the predetermined disease label is rejected, when the enrichment score for the predetermined disease label is less than or equal to a first predetermined enrichment score threshold.

Embodiment 59. The method of any of embodiments 1-58, wherein the predetermined disease label is rejected, when the confidence value is less than or equal to a first predetermined confidence value threshold.

Embodiment 60. The method of any of embodiments 1-59, wherein the predetermined disease label is rejected, when the enrichment score for the predetermined disease label is less than or equal to the first predetermined enrichment score threshold and the confidence value is less than or equal to the first predetermined confidence value threshold.

Embodiment 61. The method of any of embodiments 4-60, wherein the alternate disease label is accepted, when the enrichment score for the alternate disease label is greater than or equal to a second predetermined enrichment score threshold.

Embodiment 62. The method of any of embodiments 4-61, wherein the alternate disease label is accepted, when the confidence value is less than or equal to a second predetermined confidence value threshold.

Embodiment 63. The method of any of embodiments 4-62, wherein the alternate disease label is accepted, when the enrichment score for the predetermined disease label is greater than or equal to the second predetermined enrichment score threshold and the confidence value is less than or equal to the second predetermined confidence value threshold.

Embodiment 64. A method for diagnosing a disease, the method comprising diagnosing that the subject has a disease based on the confirming or rejecting of the predetermined disease label, wherein the confirming or rejecting of the predetermined disease label is according to the method of any of embodiments 1 to 63.

Embodiment 65. A method for diagnosing a disease, the method comprising diagnosing that the subject has a disease based on an assigning the alternate disease label, wherein the assigning the alternate disease label is according to the method of any of embodiments 4 to 64.

Embodiment 66. A method of selecting an anti-cancer therapy effective in treating a cancer, the method comprising:
responsive to confirming a predetermined disease label for the cancer according to the method of any of embodiments 1 to 65, selecting an anti-cancer therapy effective in treating the cancer.

Embodiment 67. A method of selecting an anti-cancer therapy effective in treating a cancer, the method comprising:
responsive to assigning an alternate disease label for the cancer according to the method of any of embodiments 4 to 66, selecting an anti-cancer therapy effective in treating the cancer.

Embodiment 68. A method of treating the cancer in the subject, comprising: responsive to confirming a predetermined disease label for the cancer according to the method of any one of embodiments 1 to 67, administering to the subject an anti-cancer therapy effective in treating the cancer.

Embodiment 69. A method of treating the cancer in the subject, comprising: responsive to assigning an alternate disease label for the cancer according to the method of any one of embodiments 4 to 68, administering to the subject an anti-cancer therapy effective in treating the cancer.

Embodiment 70. A method for monitoring cancer progression or recurrence in a subject, the method comprising:
confirming or rejecting a first predetermined disease label or assigning a first alternate disease label in a first sample obtained from the subject at a first time point according to the method of any of embodiments 1 to 69;
confirming or rejecting a second predetermined disease label or assigning a second alternate disease label in a second sample obtained from the subject at a second time point, and comparing the first predetermined disease label or the first alternate disease label to the second predetermined disease label or the second alternate disease label, thereby monitoring the cancer progression or recurrence.

Embodiment 71. The method of embodiment 70, wherein the second predetermined disease label or the second alternate disease label for the second sample is determined according to the method of any of embodiments 1 to 70.

Embodiment 72. The method of embodiment 70 or 71, further comprising selecting an anti-cancer therapy for the subject in response to the cancer progression.

Embodiment 73. The method of any of embodiments 70-72, further comprising administering an anti-cancer therapy to the subject in response to the cancer progression.

Embodiment 74. The method of any of embodiments 70-73, further comprising adjusting an anti-cancer therapy for the subject in response to the cancer progression.

Embodiment 75. The method of any of embodiments 72-74, further comprising adjusting a dosage of the anti-cancer therapy or selecting a different anti-cancer therapy in response to the cancer progression.

Embodiment 76. The method of embodiment 75, further comprising administering the adjusted anti-cancer therapy to the subject.

Embodiment 77. The method of any of embodiments 70-76, wherein the first time point is before the subject has been administered an anti-cancer therapy, and wherein the second time point is after the subject has been administered the anti-cancer therapy.

Embodiment 78. The method of any of embodiments 66-77, wherein the subject has a cancer, is at risk of having a cancer, is being routine tested for cancer, or is suspected of having a cancer.

Embodiment 79. The method of any of embodiments 66-78, wherein the cancer is a solid tumor.

Embodiment 80. The method of any of embodiments 66-79, wherein the cancer is a hematological cancer.

Embodiment 81. The method of any of embodiments 66-80, wherein the anti-cancer therapy comprises chemotherapy, radiation therapy, immunotherapy, a targeted therapy, or surgery.

Embodiment 82. The method of any of embodiments 1-81, further comprising determining, identifying, or applying the predetermined disease label as a diagnostic value associated with the sample.

Embodiment 83. The method of any of embodiments 4-82, further comprising determining, identifying, or applying the alternate disease label as a diagnostic value associated with the sample.

Embodiment 84. The method of any of embodiments 1-83, further comprising generating a genomic profile for the subject based on confirming or rejecting the predetermined disease label.

Embodiment 85. The method of any of embodiments 4-84, further comprising generating a genomic profile for the subject based on assigning the alternate disease label.

Embodiment 86. The method of embodiment 85, wherein the genomic profile for the subject further comprises results from a comprehensive genomic profiling (CGP) test, a gene expression profiling test, a cancer hotspot panel test, a DNA methylation test, a DNA fragmentation test, an RNA fragmentation test, or any combination thereof.

Embodiment 87. The method of embodiment 85 or 86, wherein the genomic profile for the subject further comprises results from a nucleic acid sequencing-based test.

Embodiment 88. The method of any of embodiments 85-87, further comprising selecting an anti-cancer therapy, administering an anti-cancer therapy, or applying an anti-cancer therapy to the subject based on the generated genomic profile.

Embodiment 89. The method of any of embodiments 1-88, wherein the confirming or rejecting of the predetermined disease label for the sample is used in making suggested treatment decisions for the subject.

Embodiment 90. The method of any of embodiments 4-89, wherein the assigning the alternate disease label for the sample is used in making suggested treatment decisions for the subject.

Embodiment 91. The method of any of embodiments 1-90, wherein the confirming or rejecting of the predetermined disease label for the sample is used in applying or administering a treatment to the subject.

Embodiment 92. The method of any of embodiments 4-91, wherein the assigning the alternate disease label for the sample is used in applying or administering a treatment to the subject.

Embodiment 93. A system comprising:
one or more processors; and
a memory communicatively coupled to the one or more processors and configured to store instructions that, when executed by the one or more processors, cause the system to:
   receive test sample characterization data comprising genomic alteration statuses for a test sample, wherein the test sample has a predetermined disease label;
   receive for database samples, database characterization data, wherein at least one database sample in the database samples has a predetermined disease label;
   determine similarity scores for the database samples, wherein the similarity scores indicate similarities between the test sample characterization data and the database characterization data;
   rank the database samples based on the similarity scores, to generate ranked database samples;
   select from the ranked database samples, a subset of database samples most similar to the test sample;
   determine an enrichment score for the predetermined disease label based on the subset of database samples and the database samples; and
   confirm or reject the predetermined disease label for the test sample based on the enrichment score.

Embodiment 94. The system of embodiment 93, further comprising instructions that, when executed by the one or more processors, cause the system to:
exclude, for the test sample, a genomic alteration status from the test sample characterization data, to generate reduced test sample characterization data;
exclude, for the database samples, the genomic alteration status from the database characterization data, to generate reduced database characterization data;
determine second similarity scores, wherein the second similarity scores indicate similarities between the reduced test sample characterization data, and for a corresponding database sample, the reduced database characterization data;
rank the database samples based on the second similarity scores, to generate second ranked database samples;
select from the second ranked database samples, a second subset of database samples most similar to the test sample;
determine a second enrichment score for the predetermined disease label based on the second subset of database samples and the database samples; and
confirm or reject the predetermined disease label for the test sample based on the enrichment score and the second enrichment score.

Embodiment 95. The system of embodiment 93 or 94, further comprising instructions that, when executed by the one or more processors, cause the system to:
determine one or more alternate enrichment scores for one or more alternate disease labels based on the subset of database samples and the database samples; and
reject the predetermined disease label; and
assign an alternate disease label for the test sample based on the enrichment score.

Embodiment 96. A non-transitory computer-readable storage system storing one or more programs, the one or more programs comprising instructions, which when executed by one or more processors of a system, cause the system to:
receive test sample characterization data comprising genomic alteration statuses for a test sample, wherein the test sample has a predetermined disease label;
receive for database samples, database characterization data, wherein at least one database sample in the database samples has a predetermined disease label;
determine similarity scores for the database samples, wherein the similarity scores indicate similarities between the test sample characterization data and the database characterization data;
rank the database samples based on the similarity scores, to generate ranked database samples;
select from the ranked database samples, a subset of database samples most similar to the test sample;
determine an enrichment score for the predetermined disease label based on the subset of database samples and the database samples; and
confirm or reject the predetermined disease label for the test sample based on the enrichment score.

Embodiment 97. The non-transitory computer-readable storage medium of embodiment 96, further comprising instructions that, when executed by the one or more processors, cause the system to:
determine one or more alternate enrichment scores for one or more alternate disease labels based on the subset of database samples and the database samples; and
reject the predetermined disease label; and
assign an alternate disease label for the test sample based on the enrichment score.

Embodiment 98. The non-transitory computer-readable storage medium of embodiment 96 or 97, further comprising instructions that, when executed by the one or more processors, cause the system to:
exclude, for the test sample, a genomic alteration status from the test sample characterization data, to generate reduced test sample characterization data;
exclude, for the database samples, the genomic alteration status from the database characterization data, to generate reduced database characterization data;
determine second similarity scores, wherein the second similarity scores indicate similarities between the reduced test sample characterization data, and for a corresponding database sample, the reduced database characterization data;
rank the database samples based on the second similarity scores, to generate second ranked database samples;
select from the second ranked database samples, a second subset of database samples most similar to the test sample;
determine a second enrichment score for the predetermined disease label based on the second subset of database samples and the database samples; and
confirm or reject the predetermined disease label for the test sample based on the enrichment score and the second enrichment score.

### EXAMPLES

The following examples further demonstrate to one skilled in the art how to make and use the methods and systems described herein and are not intended to limit the scope of the claimed invention.

### Example 1

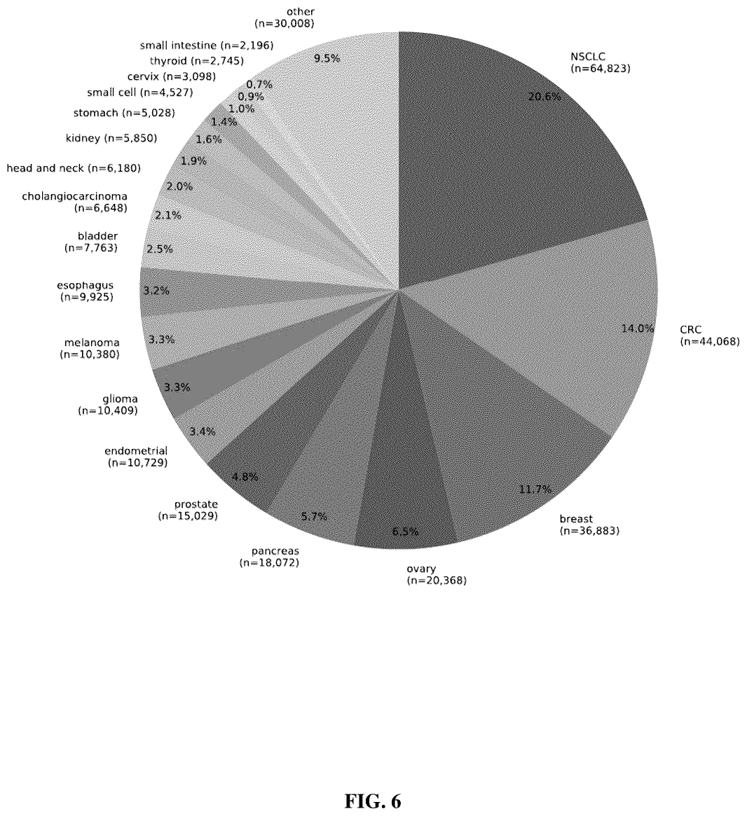

A total of 314,729 pan-solid cancer samples comprehensive genomic profile (CGP) data collected from the same number of subjects as the cancer samples (i.e., one cancer sample per subject) comprise a database against which CGP data from a sample of interest (e.g., test sample) were scored and compared, according to the embodiments of the methods disclosed herein. Duplicate cancer CGP data were removed were from the database. The subjects from which the CGP samples were procured comprised of 55.1% females and 44.8% males. FIG. 6 is a pie chart depicting the composition of cancer types for the pan-solid cancer database. The database comprises a broad spectrum of pan-solid CGP cancer data, including, but not limited to non-small cell lung cancers (NSCLCs) (n=64, 823), colorectal cancers (CRCs) (n=44, 068), breast cancers (n=36, 883), ovarian cancers (n=20, 368), pancreatic cancers (n=18, 072), prostate cancers (n=18, 072), endometrial cancers (n=10, 729), glioma, melanoma, esophageal cancers, bladder cancers, cholangiocarcinoma, head and neck cancers, kidney cancers, stomach cancers, small cell cancers, cervical cancers, thyroid cancers, and small intestinal cancers. Of these cancers, the most common cancers in the database, in order, are NSCLC, CRC, breast cancer and ovarian cancer.

CGP data for a cancer sample of interest were compared to other CGP data in the database of pan-solid cancer samples. That is, CGP data for a cancer sample of interest were selected, and the selected cancer sample of interest was compared, pairwise, to the CGP data of another database cancer sample, for all remaining database cancer samples. For all samples in this example, CGP data were processed by a genomic alteration analysis pipeline, which outputted a list of genomic alteration statuses, including putative pathogenic alteration statuses, such as specific pathogenic mutations and a tumor mutational burden (TMB) score. The list of genomic alteration statuses for a database sample of interest was then compared against all other database samples' genomic alteration statuses, to quantify the extent of genomic similarity between the database sample of interest and the other database sample. The extent of genomic similarity between two samples was quantified by adding or subtracting a value, for each genomic similarity or dissimilarity, between the two samples. The values were then summed, such that each sample-to-sample comparison had a single final score describing the two samples' genomic similarity. The final scores were then ranked from lowest to highest, and the top 800 scores were selected. From the 800 database samples corresponding to the 800 scores, the proportion of database samples that were the same cancer type as the sample of interest, versus a different type as the sample of interest, were counted, to derive a numerator value. Similarly, the number of database samples not included in the top 800 scores that were the same cancer type as the sample of interest, versus any different type as the sample of interest, were counted, to derive a denominator value. The numerator value was then divided by the denominator value, to derive an enrichment score (e.g., odds ratio) for the cancer type associated with the sample of interest. The enrichment score is a score that describes how well represented the cancer type associated with the sample of interest is, amongst the most similar (i.e., highest scoring) database samples. The four values used to derive the enrichment score for the sample of interest's cancer type—a) the number of samples of the same cancer type as the sample of interest, amongst the highest scoring database samples; b) the number of samples different from the same cancer type as the sample of interest, amongst the highest scoring database samples; c) the number of samples of the same cancer type as the sample of interest, amongst the database samples, excluding the highest scoring database samples; and d) the number of samples different from the same cancer type as the sample of interest, amongst the database samples, excluding the highest database samples-were used to also generate a Fischer's exact test-derived p-value. If the enrichment score was equal to or less than 0.5 and the p-value was equal to or less than 10-s, then the cancer type (e.g., disease group) associated with the sample of interest was considered depleted amongst the most similar database samples. In addition to computing the enrichment score for the disease group associated with the sample of interest, an enrichment score, e.g., odds ratio, for the disease ontology, e.g., the specific cancer type, associated with the sample of interest was computed. If the odds ratio for the sample of interest's disease ontology was equal to or less than 0.5, then the disease ontology associated with the sample of interest was considered depleted amongst the most similar database samples. If the odds ratio for both the sample of interest's disease group and disease ontology were equal to or less than 0.5, and the Fisher's exact test-derivedp-value for the sample's disease group was less than 10-s, then the sample of interest was considered depleted amongst the most similar database samples. Otherwise, the sample of interest was considered not depleted amongst the most similar database samples.

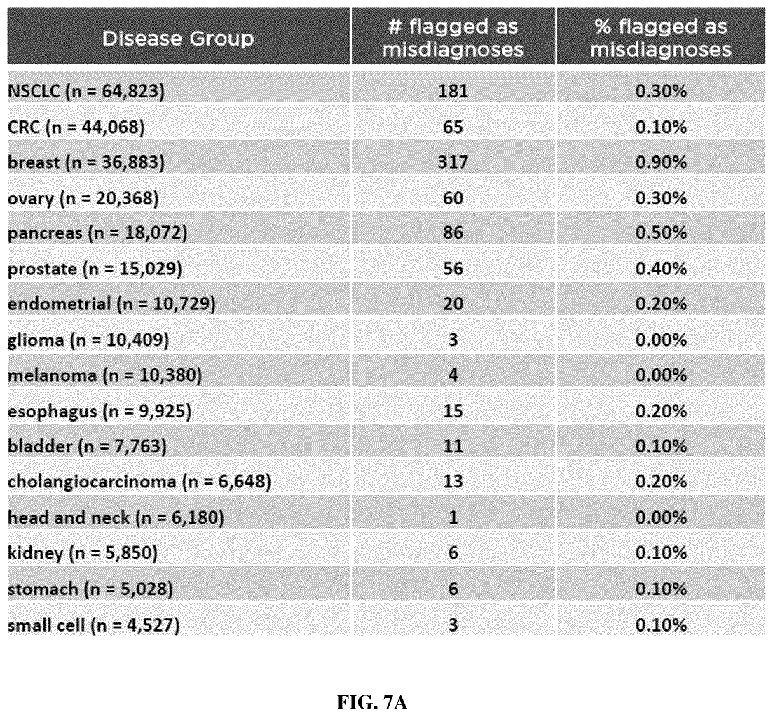

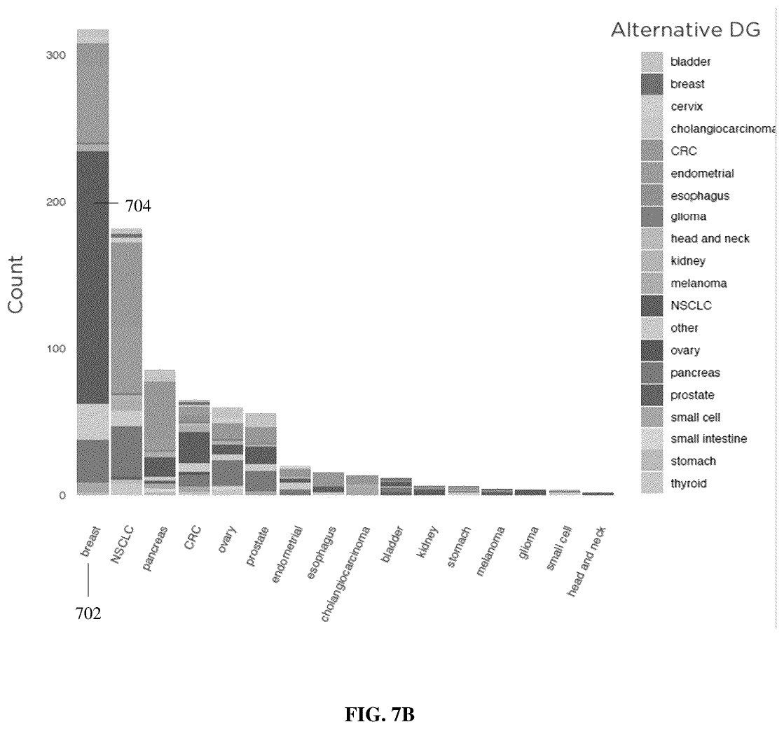

In the case that the cancer type originally associated with the sample of interest was depleted amongst the most similar database samples, then the most similar (i.e., highest scoring) database samples were examined for the possibility that the most similar database samples were enriched for alternative cancer types. Of these most similar database samples, an enrichment score for each alternative cancer type was computed. If the enrichment score was equal to or greater than 2 and the p-value was equal to or less than 10⁻⁵, then the original cancer type associated with the sample of interest was classified as a misdiagnosis, and the alternative cancer type was classified as the more likely diagnosis based on the sample of interest's CGP data. In addition to computing the enrichment score and/or the p-value based on all the genomic alteration statuses of the sample of interest and all the genomic alteration statuses of every remaining sample in the database, a genomic alteration status-contingent scoring-based method was used to assess whether the misdiagnosis classification remained intact, after a genomic alteration status was removed, when determining the scores. When determining the genomic alteration status-contingent scores, each genomic alteration status was removed, one at a time, for each comparison (i.e., between the sample of interest and every remaining sample of the 800 most similar scoring samples), before the final genomic alteration status-contingent score was computed. The case that a classified misdiagnosis remains, even after removing, in turn, each of the genomic alteration statuses, suggests that the misdiagnosis classification is not an artifact of a singular or outlier genomic alteration status providing undue weight upon the final scores. The determining of the genomic alteration status-contingent scores provides confidence, when classifying a potential diagnosis as a misdiagnosis. FIG. 7A and FIG. 7B depict the results of the method described above, wherein multiple sample of interests were compared, one at a time, against the database of samples represented in FIG. 6, to uncover a number of potential cancer misdiagnoses based on CGP data. FIG. 7A is a table depicting the number and percentage of misdiagnoses, for various disease groups, i.e., cancer types, according to methods described herein. FIG. 7B depicts the data presented in FIG. 7A as a stacked bar plot, but in addition to the information presented in FIG. 7A, the stacked bar chart in FIG. 7B reveals the composition of alternative diagnoses that constitute the misdiagnoses, for each disease group, i.e., cancer type. For example, FIG. 7A shows 317 misdiagnoses associated with the breast cancer disease group (row 3). Accordingly, in FIG. 7B, bar 702 depicts the 317 misdiagnoses, and the alternative diagnoses that comprise the 317 misdiagnoses, such as subset 704, which represents non-small cell lung cancer (NSCLC) and is the largest proportion of the breast cancer misdiagnoses. FIG. 7A and FIG. 7B reveal that not only can the methods disclosed herein identify a substantial number of diagnoses that are possibly incorrect, in light of genomic data, but that the methods described herein can also suggest the potential alternative correct diagnoses, based on the genomic data.

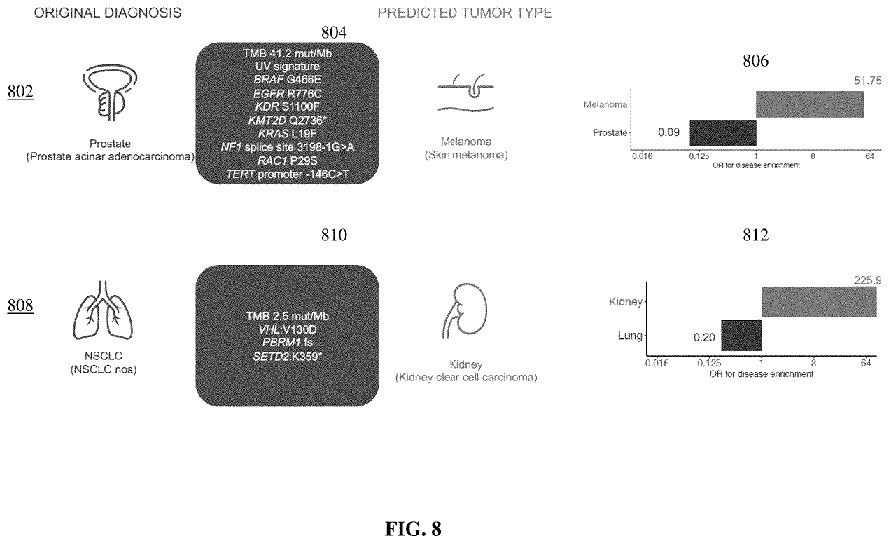

Some of the misdiagnoses depicted in FIGS. 6, 7A, and 7B were further investigated and visualized. FIG. 8 depicts exemplary data associated with two such sample of interests that were flagged as misdiagnoses. Sample of interest 802 was originally diagnosed with prostate acinar adenocarcinoma. Sample of interest 802 and its corresponding test characterization data 804 were then compared against the database of samples illustrated in FIG. 6, as described above. The test characterization data 804 included the BRAF missense mutation G466E, a UV genomic signature, and other qualities. These qualities were compared to the database of samples, to yield a subset of cancer data from the database that was considered to be the most similar to the sample of interest 802. The comparison yielded a subset of cancers with high similarity scores to the sample of interest 802, but this subset of most similar cancers comprised largely non-prostate cancers (i.e., prostate cancers received low similarity scores). The extent to which the original prostate cancer diagnosis for the sample of interest 802 was prevalent in the subset of most similar cancers from the database was quantified by calculating an odds ratio. The odds ratio was determined by calculating the normalized number of prostate cancers in the subset of cancers most similar to the sample of interest cancer (which, in this case, comprised of few prostate cancers), divided by the normalized number of prostate cancers across the entire database of samples. The odds ratio for the original prostate cancer diagnosis was small (0.09), as indicated in bar plot 806. The small odds ratio suggested that the original prostate cancer diagnosis may be less correct, i.e., bear fewer genomic similarities to the sample of interest 802, relative to some other alternative cancer diagnosis. An alternative cancer diagnosis was determined by computing genomic alteration status-contingent scores, and based on those scores, selecting the cancer corresponding to the highest odds ratio. In this case, the cancer type, e.g., disease group, with the highest odds ratio (51.75), given the genomic alteration status-contingent scores, was skin cancer (e.g., skin melanoma), as indicated in bar plot 806. Given that the odds ratio for the prostate cancer (0.09) was low, and thus suggestive of a low similarity between the original prostate cancer diagnosis and the sample of interest, an alternative diagnosis of melanoma was found (odds ratio of 51.75).

Sample of interest 808 was originally diagnosed with NSCLC (non-small cell lung cancer). Sample of interest 808 and its corresponding test characterization data 810 were then compared against the database of samples illustrated in FIG. 6, as described above. The test characterization data 810 included the VHL missense mutation V130D and the SETD2 nonsense mutation K359*. These qualities were compared to the database of samples, to yield a subset of cancer data from the database that was considered to be the most similar to the sample of interest 808. The comparison yielded a subset of cancers with high similarity scores to the sample of interest 808, but this subset of most similar cancers comprised largely non-lung cancers (i.e., lung cancers received low similarity scores). The extent to which the original lung cancer diagnosis for the sample of interest 808 was prevalent in the subset of most similar cancers from the database was quantified by calculating an odds ratio. The odds ratio was determined by calculating the normalized number of lung cancers in the subset of cancers most similar to the sample of interest cancer (which, in this case, comprised of few lung cancers), divided by the normalized number of lung cancers across the entire database of samples. The odds ratio for the original lung cancer diagnosis was small (0.20), as indicated in bar plot 812. The small odds ratio suggested that the original lung cancer diagnosis may be less correct, i.e., bear fewer genomic similarities to the sample of interest 808, relative to some other alternative cancer diagnosis. An alternative cancer diagnosis was determined by computing genomic alteration status-contingent scores, and based on those scores, selecting the cancer corresponding to the highest odds ratio. In this case, the cancer type, e.g., disease group, with the highest odds ratio (225.9), given the genomic alteration status-contingent scores, was kidney cancer (e.g., kidney clear cell carcinoma), as indicated in bar plot 812. Given that the odds ratio for the lung cancer (0.20) was low, and thus suggestive of a low similarity between the original lung cancer diagnosis and the sample of interest, an alternative diagnosis of kidney cancer was found (odds ratio of 225.9).

It should be understood from the foregoing that, while particular implementations of the disclosed methods and systems have been illustrated and described, various modifications can be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations and equivalents.

1. (canceled)

2. A method for detecting a disease type comprising:
receiving, at one or more processors, test sample characterization data comprising genomic alteration statuses for a test sample having a predetermined disease label;
receiving, at the one or more processors, for database samples, database characterization data, wherein at least one database sample in the database samples has a predetermined disease label;
determining, using the one or more processors, similarity scores for the database samples, wherein the similarity scores indicate similarities between the test sample characterization data and the database characterization data;
ranking, using the one or more processors, the database samples based on the similarity scores to generate ranked database samples;
selecting, using the one or more processors, from the ranked database samples, a subset of database samples most similar to the test sample;
determining, using the one or more processors, an enrichment score for the predetermined disease label based on the subset of database samples and the database samples; and
confirming or rejecting, using the one or more processors, the predetermined disease label for the test sample based on the enrichment score.

3. The method of claim 2, further comprising:
excluding, using the one or more processors, for the test sample, a genomic alteration status from the test sample characterization data to generate reduced test sample characterization data;
excluding, using the one or more processors, for the database samples, the genomic alteration status from the database characterization data to generate reduced database characterization data;
determining, using the one or more processors, second similarity scores, wherein the second similarity scores indicate similarities between the reduced test sample characterization data and, for a corresponding database sample, the reduced database characterization data;
ranking, using the one or more processors, the database samples based on the second similarity scores to generate second ranked database samples;
selecting, using the one or more processors, from the second ranked database samples, a second subset of database samples most similar to the test sample;
determining, using the one or more processors, a second enrichment score for the predetermined disease label based on the second subset of database samples and the database samples; and
confirming or rejecting, using the one or more processors, the predetermined disease label for the test sample based on the enrichment score and the second enrichment score.

4. The method of claim 2, further comprising:
determining, using the one or more processors, one or more alternate enrichment scores for one or more alternate disease labels based on the subset of database samples and the database samples;
rejecting, using the one or more processors, the predetermined disease label; and
assigning, using the one or more processors, an alternate disease label from the one or more alternate disease labels for the test sample based on the enrichment score.

5-37. (canceled)

38. The method of claim 2, wherein one or more of the determined similarity scores or one or more of the determined second similarity scores increase by a predetermined pathogenic short variant scoring value, when the test sample and the corresponding database sample share a pathogenic short variant affecting a same gene.

39. The method of claim 2, wherein one or more of the determined similarity scores or one or more of the determined second similarity scores increase by a predetermined same pathogenic effect scoring value, when the test sample and the corresponding database sample share a pathogenic short variant affecting a same gene with identical protein effects.

40. The method of claim 2, wherein one or more of the determined similarity scores or one or more of the determined second similarity scores increase by a predetermined pathogenic copy number amplification scoring value, when the test sample and the corresponding database sample share a pathogenic copy number amplification occurring on a same amplicon segment.

41. The method of claim 2, wherein one or more of the determined similarity scores or one or more of the determined second similarity scores increases by a predetermined pathogenic copy number deletion scoring value, when the test sample and the corresponding database sample share a pathogenic copy number deletion occurring on a same commonly deleted segment.

42. The method of claim 2, wherein one or more of the determined similarity scores or one or more of the determined second similarity scores increase by a predetermined pathogenic rearrangement scoring value, when the test sample and the corresponding database sample share a same two gene partners in a pathogenic rearrangement.

43. The method of claim 2, wherein one or more of the determined similarity scores or one or more of the determined second similarity scores increase by a predetermined same gene partner pathogenic rearrangement scoring value, when the test sample and the corresponding database sample share a same one gene partner in a pathogenic rearrangement.

44. The method of claim 2, wherein one or more of the determined similarity scores or one or more of the determined second similarity scores decrease by a predetermined non-common genomic alteration status scoring value, when the test sample and the corresponding database sample do not share a same genomic alteration status from the genomic alteration statuses.

45. The method of claim 2, wherein one or more of the determined similarity scores or one or more of the determined second similarity scores increase by a predetermined tumor mutational burden (TMB) scoring value, when the test sample and the corresponding database sample each have a TMB score above a predetermined TMB score threshold.

46. The method of claim 2, wherein one or more of the determined similarity scores or one or more of the determined second similarity scores increase by a predetermined dominant mutational signature scoring value, when the test sample and the one database sample share a dominant mutational signature.

47. (canceled)

48. The method of claim 2, wherein one or more of the determined similarity scores or one or more of the determined second similarity scores increase by a predetermined high TMB and dominant mutational signature scoring value, when the test sample and the corresponding database sample share both the high TMB score and the dominant mutational signature.

49. The method of claim 2, wherein the one or more determined similarity scores or the one or more determined second similarity scores increase by a predetermined copy number signature scoring value, when the test sample and the corresponding database sample share a copy number signature.

50. The method of claim 2, wherein the one or more determined similarity scores or the one or more determined second similarity scores increase by a predetermined aneuploidy feature scoring value, when the test sample and the corresponding database sample share a common aneuploidy feature.

51. (canceled)

52. The method of claim 2, wherein database samples corresponding to a predetermined number of most similar database samples are used for determining the enrichment score or the second enrichment score.

53-55. (canceled)

56. The method of claim 2, further comprising determining a confidence value indicating whether the predetermined disease label is correctly confirmed or rejected.

57. (canceled)

58. The method of claim 2, wherein the predetermined disease label is rejected when:
the enrichment score for the predetermined disease label is less than or equal to a first predetermined enrichment score threshold; or
the confidence value is less than or equal to a first predetermined confidence value threshold; or
the enrichment score for the predetermined disease label is less than or equal to the first predetermined enrichment score threshold and the confidence value is less than or equal to the first predetermined confidence value threshold.

59-60. (canceled)

61. The method of claim 4, wherein the alternate disease label is accepted when:
the enrichment score for the alternate disease label is greater than or equal to a second predetermined enrichment score threshold; or
the confidence value is less than or equal to a second predetermined confidence value threshold; or
when the enrichment score for the predetermined disease label is greater than or equal to the second predetermined enrichment score threshold and the confidence value is less than or equal to the second predetermined confidence value threshold.

62-65. (canceled)

66. A method of selecting an anti-cancer therapy effective in treating a cancer, the method comprising:
responsive to confirming a predetermined disease label for the cancer according to the method of claim 2, selecting an anti-cancer therapy effective in treating the cancer.

67-98. (canceled)
